(19) United States
(12) Patent Application Publication
(10) Pub. No.: US 2015/0032028 A1
(43) Pub. Date: Jan. 29, 2015

(54) SYSTEMS AND METHODS FOR TREATMENT OF AN AIRWAY DISORDER

(71) Applicants: Charles R. RAMPERSAUD, Castro Valley, CA (US); Edward M. GILLIS, Livermore, CA (US); Michael D. FOSTER, East Palo Alto, CA (US); Emmanuelle F. PEASE, San Francisco, CA (US)

(72) Inventors: Charles R. RAMPERSAUD, Castro Valley, CA (US); Edward M. GILLIS, Livermore, CA (US); Michael D. FOSTER, East Palo Alto, CA (US); Emmanuelle F. PEASE, San Francisco, CA (US)

(21) Appl. No.: 14/340,324

(22) Filed: Jul. 24, 2014

Related U.S. Application Data
(60) Provisional application No. 61/857,814, filed on Jul. 24, 2013.

(51) Int. Cl. A61F 5/56; A61F 2/00; A61B 17/34; A61B 17/00; A61B 17/22; A61M 25/09; A61B 1/06

(52) U.S. Cl. CPC A61F 5/56; A61M 25/09016; A61F 2/0063; A61B 1/06; A61B 17/00234; A61B 17/22; A61B 17/3468; A61M 2025/09175; A61B 2017/00336; A61B 2017/22042; A61F 2002/0072
USPC 600/585; 128/848

(57) ABSTRACT

Methods and systems for treating an airway disorder and delivering an implant to airway tissue are provided. A method includes partially inserting at least a first and second wire into airway forming tissue, wherein the first and second wires' axes define implant positions for a first and second implant, respectively. A wire guide can be used to maintain an orientation of the first and second axis relative to each other.

### CROSS-REFERENCE TO RELATED APPLICATIONS

The present application claims the benefit of U.S. Provisional Patent Application Ser. No. 61/857,814, filed Jul. 24, 2013, the disclosure of which is hereby incorporated by reference in its entirety.

### INCORPORATION BY REFERENCE

This application may be related to any of the following applications: application Ser. No. 11/969,201, issued as U.S. Pat. No. 8,167,787, filed Jan. 3, 2008, entitled PARTIALLY ERODABLE SYSTEMS FOR TREATMENT OF OBSTRUCTIVE SLEEP APNEA; application Ser. No. 13/443,839, entitled PARTIALLY ERODABLE SYSTEMS FOR TREATMENT OF OBSTRUCTIVE SLEEP APNEA, filed Apr. 10, 2012; application Ser. No. 61/052,586, filed May 12, 2008, entitled PARTIALLY ERODABLE SYSTEMS FOR TREATMENT OF OBSTRUCTIVE SLEEP APNEA; application Ser. No. 13/269,520, issued as U.S. Pat. No. 8,327,854, filed Oct. 7, 2011, entitled PARTIALLY ERODABLE SYSTEMS FOR TREATMENT OF OBSTRUCTIVE SLEEP APNEA; application Ser. No. 13/711,537, filed Dec. 11, 2012, entitled PARTIALLY ERODABLE SYSTEMS FOR TREATMENT OF OBSTRUCTIVE SLEEP APNEA; application Ser. No. 12/937,564, filed Jan. 3, 2011, entitled PARTIALLY ERODABLE SYSTEMS FOR TREATMENT OF OBSTRUCTIVE SLEEP APNEA; App. No. 61/315,835, filed Mar. 19, 2010, entitled SYSTEMS AND METHODS FOR TREATMENT OF SLEEP APNEA; application Ser. No. 13/053,025, filed Mar. 21, 2011, entitled SYSTEMS AND METHODS FOR TREATMENT OF SLEEP APNEA; App. No. 61/315,838, filed Mar. 19, 2010, entitled SYSTEMS AND METHODS FOR TREATMENT OF SLEEP APNEA; application Ser. No. 13/053,059, filed Mar. 21, 2011, entitled SYSTEMS AND METHODS FOR TREATMENT OF SLEEP APNEA; App. No. 61/347,348, filed May 21, 2010, entitled SYSTEMS AND METHODS FOR TREATMENT OF SLEEP APNEA; application Ser. No. 13/113,933, filed May 23, 2011, entitled SYSTEMS AND METHODS FOR TREATMENT OF SLEEP APNEA; App. No. 61/347,356, filed May 21, 2010, entitled SYSTEMS AND METHODS FOR TREATMENT OF SLEEP APNEA; application Ser. No. 13/113,946, filed May 23, 2011, entitled SYSTEMS AND METHODS FOR TREATMENT OF SLEEP APNEA; App. No. 61/367,707, filed Jul. 26, 2010, entitled SYSTEMS AND METHODS FOR TREATMENT OF SLEEP APNEA; application Ser. No. 13/188,385, filed Jul. 21, 2011, entitled SYSTEMS AND METHODS FOR TREATMENT OF SLEEP APNEA; App. No. 61/418,238, filed Nov. 30, 2010, entitled SYSTEMS AND METHODS FOR TREATMENT OF SLEEP APNEA; application Ser. No. 13/308,449, filed Nov. 30, 2011, entitled SYSTEMS AND METHODS FOR TREATMENT OF SLEEP APNEA; App. No. 61/419,690, filed Dec. 3, 2010, entitled SYSTEMS AND METHODS FOR TREATMENT OF SLEEP APNEA; application Ser. No. 13/311,460, filed Dec. 5, 2011, entitled SYSTEMS AND METHODS FOR TREATMENT OF SLEEP APNEA; application Ser. No. 13/539,081, filed Jun. 29, 2012, entitled SYSTEMS AND METHODS FOR TREATMENT OF SLEEP APNEA; App. No. 61/671,643, filed Jul. 13, 2012, entitled SYSTEMS AND METHODS FOR TREATMENT OF SLEEP APNEA; application Ser. No. 13/939,107, filed Jul. 10, 2013, entitled SYSTEMS AND METHODS FOR TREATMENT OF SLEEP APNEA; App. No. 61/668,991, filed Jul. 6, 2012, entitled SYSTEMS AND METHODS FOR TREATMENT OF SLEEP APNEA; application Ser. No. 13/935,052, filed Jul. 3, 2013, entitled SYSTEMS AND METHODS FOR TREATMENT OF SLEEP APNEA.

All publications and patent applications mentioned in this specification are herein incorporated by reference to the same extent as if each individual publication or patent application was specifically and individually indicated to be incorporated by reference.

### FIELD

This invention relates to the field of methods and devices for the treatment of airway disorders such as obstructive sleep apnea, and more particularly to opening the airway of subjects with systems of any obstructive airway disorder.

### BACKGROUND

Sleep apnea is defined as the cessation of breathing for ten seconds or longer during sleep. During normal sleep, the throat muscles relax and the airway narrows. During the sleep of a subject with obstructive sleep apnea (OSA), the upper airway narrows significantly more than normal, and during an apneic event, undergoes a complete collapse that stops airflow. In response to a lack of airflow, the subject is awakened at least to a degree sufficient to reinitiate breathing. Apneic events and the associated arousals can occur up to hundreds of times per night, and become highly disruptive of sleep. Obstructive sleep apnea is commonly but not exclusively associated with a heavy body type, a consequence of which is a narrowed oropharyngeal airway.

Cyclic oxygen desaturation and fragmented sleeping patterns lead to daytime sleepiness, the hallmark symptom of the disorder. Further consequences of sleep apnea may include chronic headaches and depression, as well as diminished facilities such as vigilance, concentration, memory, executive function, and physical dexterity. Ultimately, sleep apnea is highly correlated with increased mortality and life threatening co-morbidities. Cardiology complications include hypertension, congestive heart failure, coronary artery disease, cardiac arrhythmias, and atrial fibrillation. OSA is a highly prevalent disease conditions in the United States. An estimated 18 million Americans suffer from OSA to degrees that range from mild to severe, many of whom are undiagnosed, at least in part because the afflicted subjects are often unaware of their own condition.

Treatment of OSA usually begins with suggested lifestyle changes, including weight loss and attention to sleeping habits (such as sleep position and pillow position), or the use of oral appliances that can be worn at night, and help position the tongue away from the back of the airway. More aggressive physical interventions include the use of breathing assist systems that provide a positive pressure to the airway through a mask that the subject wears, and which is connected to a breathing machine. In some cases, pharmaceutical interventions can be helpful, but they generally are directed toward countering daytime sleepiness, and do not address the root cause.

Additionally, some surgical interventions are available, such as nasal surgeries, tonsillectomy and/or adenoidectomy, reductions in the soft palate or the uvula or the tongue base, or advancing the tongue base by an attachment to the mandible and pulling the base forward. These surgical approaches can be quite invasive and thus have a last-resort aspect to them, and further, simply do not reliably alleviate or cure the condition.

Another challenge to surgical intervention has been the need for placing multiple implants in a target site for effective treatment. Because an airway disorder can manifest in a myriad of ways that include weakening of any airway structures and compromise of any airway passages, the number of implants and the placement of the implants in tissue can greatly affect therapeutic efficacy. As can be appreciated, placing multiple implants carries the added challenges of minimizing tissue trauma with repeated implant delivery as well as delivering implants in appropriate relative placement to optimize therapeutic effect.

While conventional implant delivery techniques are applicable for multiple implant delivery, these are not ideal given the particular challenges of multi-implant treatment. For example, conventionally, an introducer such as a trocar may be inserted into tissue for delivery of a first implant. Once the first implant is delivered, the trocar may be inserted again for delivery of a second implant. However, imaging (e.g. x-ray or fluoroscopy) may be required to confirm the position of the trocar in the tissue relative to the first implant. This allows the proper positioning of the second implant relative to the first. The trade-off is the patient's exposure to multiple sessions of x-ray imaging that carries its own side-effects.

As such, there is a need for less invasive procedures that show promise for greater therapeutic reliability, particularly for multi-implant therapy. Embodiments described herein address at least these concerns.

### SUMMARY OF THE DISCLOSURE

In one aspect, a system for delivering an implant into a patient's airway tissue is provided. The system comprises at least one wire comprising a first wire having a first distal end, first proximal end, and a first wire axis configured to define a first implant position in the airway tissue for a first implant, and the first distal end configured to allow at least partial insertion of the first wire into the airway tissue, wherein when partially inserted a proximal portion of the first wire remains outside the patient's body. The system comprises a wire guide comprising a second distal end, a second proximal end, and at least a first wire channel and a second wire channel, each wire channel having a proximal opening, a distal opening, and a lumen extending between the openings, the first and second wire channels are each configured to releasably receive and retain a wire, and the first wire channel having a first channel axis and the second wire channel having a second channel axis, wherein the first channel axis and second channel axis extend lengthwise through the first and second wire channels respectively.

In another aspect, a system for delivering an implant into a patient's airway tissue is provided. The system comprises at least one wire comprising a first wire having a first distal end, first proximal end, and a first axis configured to define a first implant position in the airway tissue for a first implant, and the first distal end configured to allow at least partial insertion of the first wire into the airway tissue, wherein when partially inserted a proximal portion of the first wire remains outside the patient's body. The system comprises a sheath and dilator assembly comprising a dilator having a second proximal end, a second distal end, and a dilator lumen extending through the dilator between the second proximal and distal ends, wherein the first wire is configured to be movably positioned in the dilator lumen to guide the advancement of the sheath and dilator assembly into the airway tissue; and a sheath having a third proximal end, a third distal end, and a sheath lumen extending between the third proximal and distal ends, the sheath configured to removably receive the dilator into the sheath lumen, wherein the second distal end of the dilator extends beyond the third distal end of the sheath as the sheath and dilator assembly is advanced into the airway tissue. The system also comprises a pusher having a fourth proximal end and a fourth distal end, the fourth distal end configured to releasably engage a distal portion of the first implant, wherein the pusher is configured to move through the sheath lumen and deploy the first implant in the first implant position in the airway tissue.

In another aspect, a system for delivering an implant into airway tissue is provided. The system comprises a sheath and dilator assembly comprising a dilator having a proximal end, a distal end, a dilator shaft extending through the dilator between the proximal and distal ends, and a tip portion at the distal end of the dilator shaft, wherein the tip portion comprises an eccentric tip configured to guide the advancement of the sheath and dilator assembly through the airway tissue. The system also comprises a sheath having a proximal end, a distal end, and a sheath lumen extending between the proximal and distal ends, the sheath configured to removably receive the dilator into the sheath lumen, wherein the tip portion of the dilator extends beyond the distal end of the sheath as the sheath and dilator assembly is advanced into the airway tissue.

In yet another aspect, a method of treating an airway disorder is provided. The method comprises creating an incision on a surface of a tissue near an airway forming tissue and partially inserting at least a first wire and a second wire into the airway forming tissue through the incision, wherein a first axis of the first wire defines a first implant position for a first implant in the airway forming tissue and a second axis of the second wire defines a second implant position for the second implant in the airway forming tissue, the first axis and the second axis forming an angle between about 0 degrees to about 45 degrees. The method comprises placing a first implant at the first position in the airway forming tissue by guiding the first implant to the first implant position along a first path defined by the first axis of the first wire; placing a second implant at the second position in the airway forming tissue by guiding the second implant along a second path defined by the second axis of the second wire; and removing the first and second wires from the airway forming tissue.

In another aspect, a method of treating an airway disorder is provided. The method comprises creating an incision on a surface of a tissue near an airway forming tissue and partially inserting a first wire and a second wire into the airway forming tissue through the incision, wherein a first axis of the first wire defines a first implant position for a first implant in the airway forming tissue and a second axis of the second wire defines a second implant position for the second implant in the airway forming tissue. The method also comprises guiding an implant delivery device through the incision and at least partially into the airway forming tissue by advancing the delivery device along a length of the first wire; placing a first implant at the first position in the airway forming tissue; and removing the implant delivery device from the airway forming tissue after placing the first implant in the first position. The method comprises guiding the implant delivery device through the incision and at least partially into the airway forming tissue by advancing the delivery device along a length of the second wire; placing a second implant at the second position in the airway forming tissue; and removing the implant delivery device from the airway forming tissue after placing the second implant in the second position.

In another aspect, a method of treating an airway disorder is provided. The method comprises creating an incision on a surface of a tissue near an airway forming tissue; advancing a sheath and dilator assembly through the incision and at least partially into the airway forming tissue, wherein the dilator comprises an eccentric tip configured to guide the assembly along a curved area of the airway forming tissue; placing a first implant at the first position in the airway forming tissue; placing a second implant at the second position in the airway forming tissue; and removing the assembly from the airway forming tissue.

In another aspect, a system for delivering an implant into a patient's airway tissue is provided. The system comprises at least one wire comprising a first wire having a first distal end, first proximal end, and a first axis configured to define a first implant position in the airway tissue for a first implant, and the first distal end configured to allow at least partial insertion of the first wire into the airway tissue, wherein when partially inserted a proximal portion of the first wire remains outside the patient's body. The system comprises a sheath and dilator assembly comprising a dilator having a second proximal end, a second distal end, and a dilator lumen extending through the dilator between the second proximal and distal ends, wherein the first wire is configured to be movably positioned in the dilator lumen to guide the advancement of the sheath and dilator assembly into the airway tissue; and a sheath having a third proximal end, a third distal end, and a sheath lumen extending between the third proximal and distal ends, the sheath configured to removably receive the dilator into the sheath lumen, wherein the second distal end of the dilator extends beyond the third distal end of the sheath as the sheath and dilator assembly is advanced into the airway tissue.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 provides an overview of the healthy human airway anatomy, with particular attention to the nasopharyngeal, oropharangeal, and hypopharyngeal regions.

FIG. 2A provides a view of a compromised airway, with an occlusion in the oropharyngeal region due to posterior slippage of the base of the tongue.

FIG. 2B provides a view of a compromised airway with palate closure.

FIG. 3A depicts an elongate implant component of a revisable OSA implant system, the implant having end portions with openings for growth of a tissue plug therethrough to secure the end portions in a treatment site.

FIG. 3B is a cut-away view of an end portion of the implant of FIG. 3A in a tissue site.

FIG. 3C depicts another elongate implant embodiment similar to that of FIG. 3A.

FIG. 3D depicts another elongate implant embodiment.

FIG. 4 depicts another elongate implant corresponding to aspects of the invention.

FIG. 5A depicts a second component of a revisable OSA implant system, the second component comprising a cutting tool.

FIG. 5B depicts the cutting tool of FIG. 5A in a method of use.

FIG. 6 depicts an alternative cutting tool similar to that of FIGS. 5A-5B.

FIG. 7A depicts another elongate implant corresponding to aspects of the invention.

FIG. 7B depicts another elongate implant embodiment.

FIG. 7C depicts another elongate implant embodiment.

FIG. 7D depicts another elongate implant embodiment with multiple openings in multiple planes.

FIG. 7E is a partially cut-away view that depicts an OSA implant with an elastomeric portion that is configured for being releaseably maintained in a tensioned or non-repose condition by a magnesium or magnesium alloy biodissolvable material or element.

FIG. 8A depicts the working end of another embodiment of a cutting tool for cutting a portion of an implant in situ.

FIG. 8B depicts another embodiment of a cutting tool for cutting an implant in a revision procedure.

FIG. 9 depicts another implant with a medial portion having a surface configured for low adhesive energy.

FIG. 10 depicts another elongate implant corresponding to aspects of the invention.

FIG. 11 depicts another implant corresponding to aspects of the invention including a sacrificial portion that can be sacrificed in response to an external stimulus.

FIG. 12 is a cut-away view depicting the implant of FIG. 11 in a tissue site after actuation of the sacrificial portion of the implant.

FIG. 13A depicts an alternative implant including an electrolytically sacrificial portion that can be sacrificed in response to a direct current.

FIG. 13B is a cut-away view depicting the implant of FIG. 13A in a tissue site after actuation of electrolytic connection portion of the implant.

FIG. 14 depicts an end portion of an alternative revisable implant including a cut wire for cutting a tissue plug.

FIG. 15 is a cut-away view depicting the implant of FIG. 14 in a tissue site in the process of actuating the cut wire.

FIG. 16 depicts an end portion of an alternative revisable implant including a cut wire for cutting a plurality of tissue plugs.

FIG. 17 depicts an alternative revisable OSA implant.

FIGS. 18A and 18B illustrate an end portion of the revisable implant of FIG. 17.

FIG. 19 depicts an alternative revisable OSA implant.

FIG. 20 depicts a revisable OSA implant that allows for in-situ post-implant adjustment of the retraction forces applied to tissue by the implant.

FIG. 21 depicts an alternative revisable OSA implant that allows for in-situ post-implant adjustment of the retraction forces.

FIGS. 22 and 23 depict another revisable OSA implant that allows for in-situ post-implant adjustment of the retraction forces.

FIG. 24 depicts an OSA implant with first and second anchoring ends implanted in a particular site in a patient's tongue.

FIG. 25 depicts the OSA implant of FIG. 24 implanted in another particular site in a patient's tongue.

FIGS. 26-27 depict a plurality of OSA implants each with first and second anchoring ends implanted in a patient's tongue for applying linear-directed forces in different distinct vectors.

FIGS. 28A, 28B and 28C depict another OSA implant system for applying linear-directed forces in different distinct vectors with individual implant bodies coupled together in-situ with attachment means.

FIGS. 29A and 29B depict another OSA implant system similar to that of FIGS. 28A-28C for applying linear-directed forces in different distinct vectors in a different orientation.

FIG. 30 illustrates a method of utilizing a cannula apparatus for deployment of an OSA implant as in FIG. 24 in a particular site in a patient's tongue.

FIG. 31 illustrates a working end of the cannula apparatus of FIG. 30 together with a push rod or stylette mechanism for deployment of the OSA implant of FIG. 24.

FIGS. 32A and 32B illustrate a method of utilizing an alternative telescoping cannula apparatus for deployment of an OSA implant at a selected angle in a patient's tongue.

FIG. 33 illustrates another method of utilizing a cannula apparatus to penetrate through a patient's skin for deployment of an OSA implant in a patient's tongue.

FIG. 34 illustrates another method of utilizing a curved cannula apparatus for deployment of an OSA implant in a patient's tongue.

FIG. 35A illustrates two elongated implants in a patient's tongue wherein the implant orientations are non-parallel.

FIG. 35B illustrates a different view of the two elongated implants of FIG. 35A from a different perspective.

FIG. 36 illustrates two elongated implants in a patient's tongue wherein the implant orientations are asymmetric relative to the patient's mid-line.

FIG. 37 illustrates two elongated implants in a patient's soft palate wherein the implant orientations are parallel and symmetric relative to the patient's mid-line.

FIG. 38 illustrates two elongated implants in a patient's soft palate wherein the implant's axes converge in the posterior direction.

FIG. 39 illustrates two elongated implants in a patient's soft palate wherein the implant's axes diverge in the posterior direction.

FIG. 40 illustrates two elongated implants in a patient's soft palate wherein the implant's axes parallel and angled relative to the patient's mid-line.

FIG. 41 illustrates two elongated implants in a patient's soft palate wherein the implant's axes cross about the patient's mid-line.

FIG. 42 illustrates a trocar implant delivery device and method.

FIG. 43 illustrates a trocar device with a pusher for delivering implants.

FIG. 44 illustrates an implant delivered in the tongue using the trocar and pusher of FIG. 43.

FIG. 45 illustrates a wire or stylet according to described embodiments.

FIGS. 46A-B illustrates partial insertion of wires into the patient's tongue for implant delivery.

FIG. 47 illustrates the positioning of a plurality of wires relative to each other and corresponding implant positions.

FIG. 48 illustrates a plurality of wire axes defining corresponding implant positions.

FIG. 49 illustrates a plurality of implants in the implant positions defined by the wires shown in FIG. 46B.

FIGS. 50A-B show variations of implant positions.

FIG. 51 illustrates a wire guide according to some embodiments.

FIG. 52 illustrates a wire guide having three wire guide channels with respective channel axes.

FIG. 53 illustrates the relative position of the three wire guide channels for the wire guide shown in FIG. 52.

FIG. 54 illustrates a wire guide having four channels.

FIG. 55 shows an open side of the wire guide shown in FIG. 54.

FIG. 56 shows the proximal end of the wire guide in FIG. 54.

FIG. 57 shows wires positioned in the channels of the wire guide in FIG. 54.

FIG. 58 shows the open side the wire guide of FIG. 54 with four wires positioned in the wire guide channels.

FIG. 59 shows the open side of the wire guide in FIG. 54 without wires.

FIG. 60 illustrates another embodiment of a wire guide with a common channel opening.

FIG. 61 illustrates a tissue template with four position indicators.

FIG. 62 illustrates a tissue template with three position indicators.

FIG. 63 illustrates a tissue plate with a position indicator.

FIG. 64 illustrates the tissue plate of FIG. 63 placed over a patient's tongue.

FIG. 65 illustrates a sheath and dilator assembly according to described embodiments.

FIG. 66 shows the sheath of the assembly shown in FIG. 65.

FIG. 67 shows the dilator of the assembly shown in FIG. 65.

FIG. 68 shows an example of a delivery system with a pusher, wire, and sheath and dilator assembly.

FIG. 69 illustrates the insertion of a sheath and dilator assembly over a wire and into the patient.

FIG. 70 illustrates the cross-section of the sheath and dilator assembly when passed over the wire in FIG. 69.

FIG. 71 illustrates the removable of the dilator and wire from the patient in the example shown in FIG. 69.

FIG. 72 illustrates the insertion of a pusher and implant into the sheath of FIG. 69.

FIG. 73 illustrates the removal of the sheath and pusher from the patient after implant deployment.

FIG. 74 illustrates the relative positioning of two remaining wires and an implant in the patient's tongue following the removal of the sheath and pusher in FIG. 73.

FIG. 75 illustrates the insertion of wire or stylet into the soft palate.

FIG. 76 illustrates sheath and dilator assembly moved over the wire of FIG. 75 into the soft palate.

FIG. 77 illustrates the sheath in the soft palate with the dilator removed from the assembly of FIG. 76.

FIGS. 78A-B show one and two implants deployed in the soft palate respectively.

FIG. 79 illustrates a sheath and dilator assembly with an eccentric dilator tip.

FIG. 80 shows a variation of an eccentric dilator tip.

FIG. 81 shows an eccentric dilator tip according to described embodiments.

FIG. 82 shows a cross-section of the dilator tip in FIG. 81.

FIG. 83 shows a sheath and dilator assembly having the eccentric tip of FIG. 81 inserted in the soft palate.

FIG. 84 shows a variation of an eccentric dilator tip.

FIG. 85 shows a variation of an eccentric dilator tip.

FIG. 86 illustrates a wire having a light emitter.

FIG. 87 illustrates a wire having an internal light channel.

FIG. 88 illustrates a sheath and dilator assembly equipped with light guidance.

FIGS. 89A-89L are x-ray images and photos for the cadaver study described in Example 3.

### DETAILED DESCRIPTION

### A. Anatomy of the Pharynx

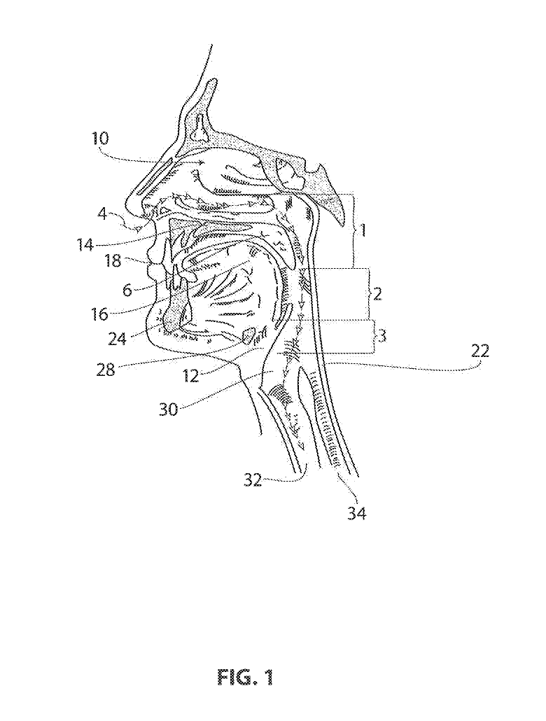

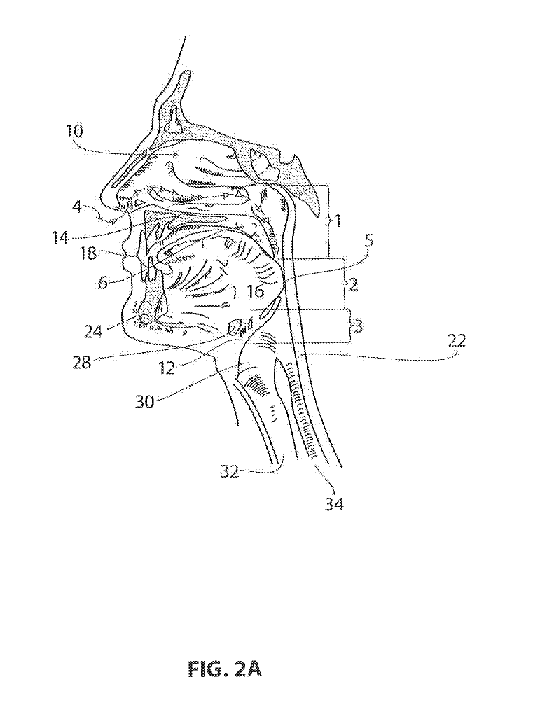

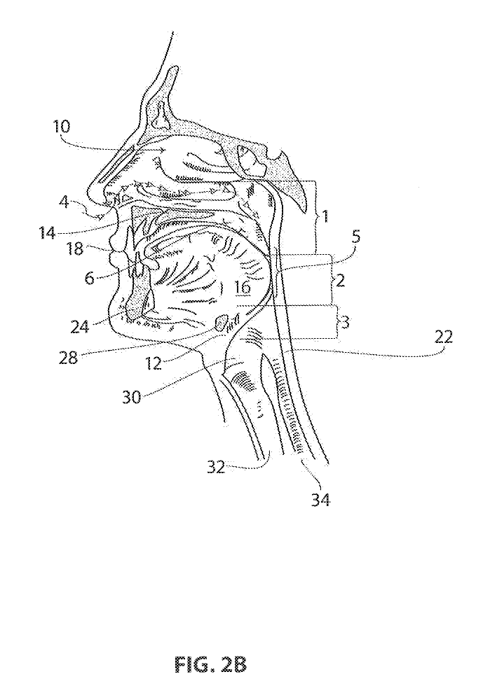

FIG. 1 is a sagittal view of the structures that form the pharyngeal airway 4; some of these structures can become compromised under various conditions to the extent that they obstruct or occlude passage of air through the airway 4, and thus contribute to obstructive sleep apnea. The pharynx is divided, from superior to inferior, into the nasopharynx 1, the oropharynx 2 and the hypopharynx 3. Variations of FIG. 1 are provided in FIGS. 2A and 2B which depict airway obstruction sites 5 at various levels in the pharyngeal airway. FIG. 2A, for example, shows an occlusion 5 at the level of the oropharynx 2, where the base of the tongue 16 and a thickened posterior pharyngeal wall 22 have collapsed against each other. FIG. 2B provides a view of a compromised airway with palate closure. It is also possible for airway obstruction to occur at the level of the nasopharynx 1, where an elongated and/or floppy soft palate can collapse against a thickened posterior pharyngeal wall. Further, an obstruction can occur at the level of the hypopharynx 3, where both an elongated soft palate and a floppy epiglottis can collapse against the pharyngeal wall 22.

With reference to FIGS. 1-2B, the nasopharynx is the portion of the pharynx at the level or above the soft palate 6. In the nasopharynx, a deviated nasal septum or enlarged nasal turbinates may occasionally contribute to upper airway resistance or blockage. Rarely, a nasal mass, such as a polyp, cyst or tumor may be a source of obstruction. The oropharynx 2 includes structures from the soft palate 6 to the upper border of the epiglottis 12 and includes the inferior surface of the hard palate 14, tongue 16, the posterior pharyngeal wall 22 and the mandible 24 as well as the tonsils and palatoglossal arch. The mandible typically has a bone thickness of about 5 mm to about 10 mm anteriorly with similar thicknesses laterally. An obstruction in the oropharynx 2 may result when the tongue 16 is displaced posteriorly during sleep as a consequence of reduced muscle activity during deep or non-REM sleep. The displaced tongue 16 may push the soft palate 6 posteriorly and may seal off the nasopharynx 1 from the oropharynx 2. The tongue 16 may also contact the posterior pharyngeal wall 22, which causes further airway obstruction.

The hypopharynx 3 includes the region from the upper border of the epiglottis 12 to the inferior border of the cricoid cartilage. The hypopharynx 3 further includes the hyoid bone 28, a U-shaped, free-floating bone that does not articulate with any other bone. The hyoid bone 28 is attached to surrounding structures by various muscles and connective tissues. The hyoid bone 28 lies inferior to the tongue 16 and superior to the thyroid cartilage 30. A thyrohyoid membrane and a thyrohyoid muscle attaches to the inferior border of the hyoid 28 and the superior border of the thyroid cartilage 30. The epiglottis 12 is infero-posterior to the hyoid bone 28 and attaches to the hyoid bone by a median hyoepiglottic ligament. The hyoid bone attaches anteriorly to the infero-posterior aspect of the mandible 24 by the geniohyoid muscle.

### B. Revisable OSA Implants

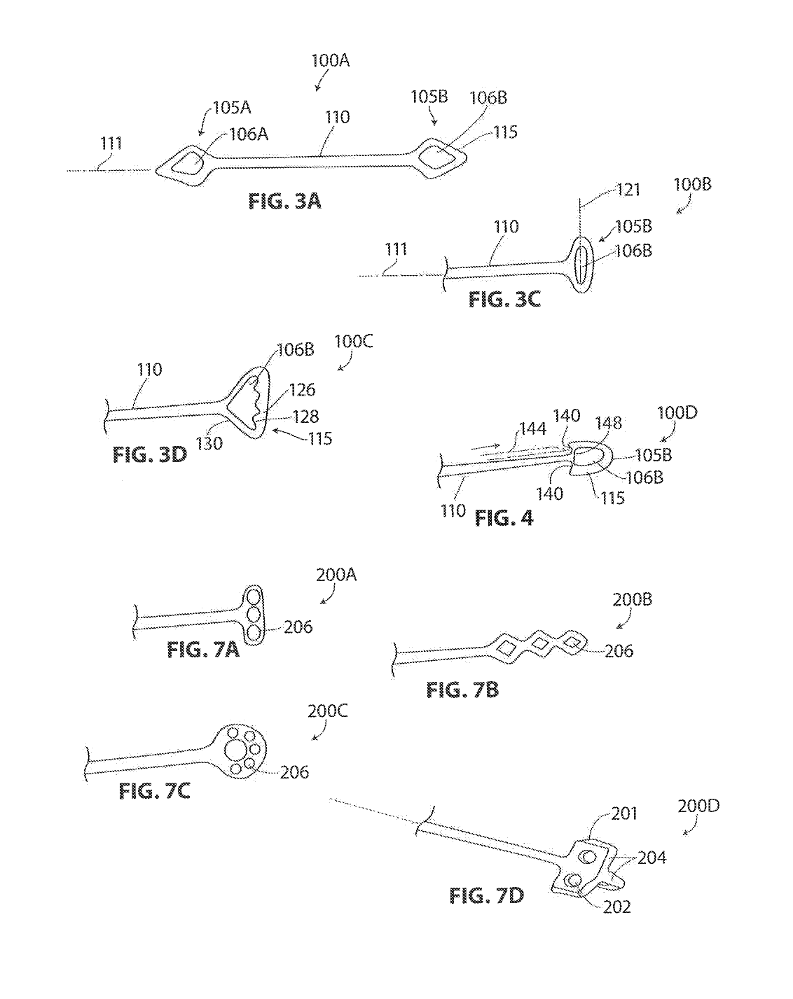

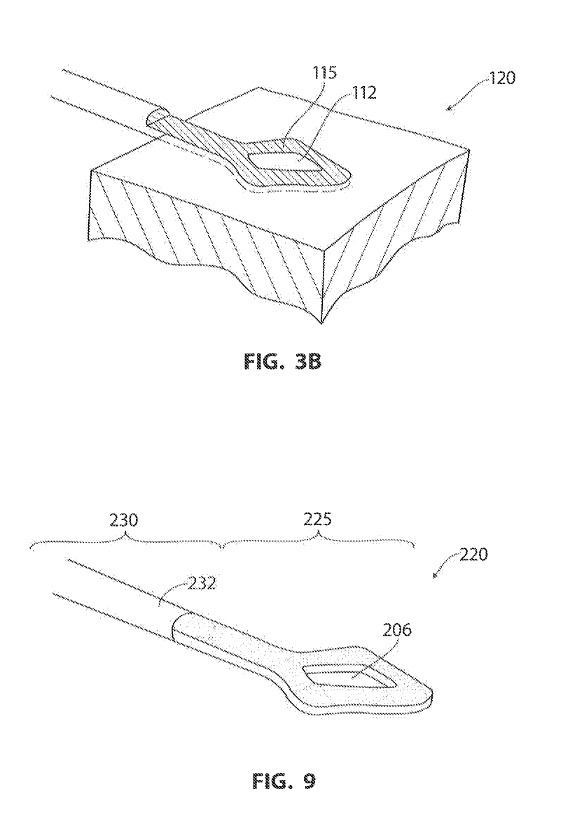

FIG. 3A depicts a first component of a kit or system that provides revisable implants for treating an airway disorders or obstructive sleep apnea (OSA). The second component of the kit is an introducer for insertion into a treatment site as is known in the art and co-pending applications. In FIG. 3A, an elongate device or implant body 100A has first and second end portions 105A and 105B with through-openings 106A and 106B therein. The medial portion 110 of the implant body 100A extends along axis 111 and comprises a biocompatible elastomeric material such as a silicone. The mean cross-section of the medial body portion 110 can range from 1 to 10 mm² and can be round, oval flat or polygonal. The elastic modulus of the medial portion can range from 0.5 to 10 MPA and is configured for implanting in the patient's airway tissue (i.e. tissue in the vicinity of the patient's airway) in a releasable, tensioned position, as described in co-pending U.S. patent application Ser. No. 11/969,201, which is incorporated herein by this reference.

Referring to FIGS. 3A and 3B, it can be seen that through-openings 106A and 106B in the implant body 100 are configured for growth of a tissue plug 112 through the opening to thereby secure the first and second end portions 105A and 105B in a selected tissue site. The cut-away view of FIG. 3B schematically illustrates that a tissue plug 112 that grows through the opening is thus surrounded or encircled by an encircling body portion 115 of the implant. The encircling body portion 115 comprises a small cross-section element that can be cut, severed, sacrificed, decoupled, or dissolved to disengage the implant from a tissue site 120 as will be described below. The element can be a polymer or other material. In other embodiments described below, the tissue plug 112 can be cut or severed to disengage the implant from the tissue site 120. In one embodiment, the mean cross-section of the tissue plug 112, and thus the dimension across an opening 106A or 106B, can range from about 0.5 mm to 10 mm or more. The openings 106A or 106B can have a round shape in plan view or any other plan shape. The end portions 105A and 105B can have similar or dissimilar configurations, for example an implant configured for treatment of a patient's tongue may have a substantially larger end portion and opening 106B for the base of the tongue and a smaller end portion near the mandible.

FIG. 3C illustrates another implant body 100B with an end portion 105B having an elongated opening 106B through which tissue will grow to form a tissue plug to secure the end portion in the site. For example, the implant body 100B of FIG. 3C has an opening 106B with a primary axis 121 and larger dimension that extends generally orthogonal to the axis 111 of medial portion 110 of the implant body. In use, the greater dimension of the tissue plug will better resist the retraction forces applied to tissue by the elastomeric medial portion 110 of the implant aligned with axis 111.

FIG. 3D depicts another embodiment 100C of a revisable implant for treating an airway disorder that is similar to that of FIG. 3C except the end portion 105B has a through-opening 106B with a terminal part 126 of encircling portion 115 configured with irregular shaped surface features 128 that can interface with the tissue plug that grows through opening 106B. The surface features can comprise undulations, textures, protrusions, bumps and the like that can assist in maintaining the end portion in a fixed position when under the tensioning or retraction forces applied by the medial portion 110 of the implant body 100C. In the implant body 100C of FIG. 3D, the end portion 105B also can have an encircling element 115 that includes a proximal portion 130 of a lower modulus material similar to the modulus of medial portion 110 and the terminal part 126 having a higher modulus to prevent it deformation under tensioning forces.

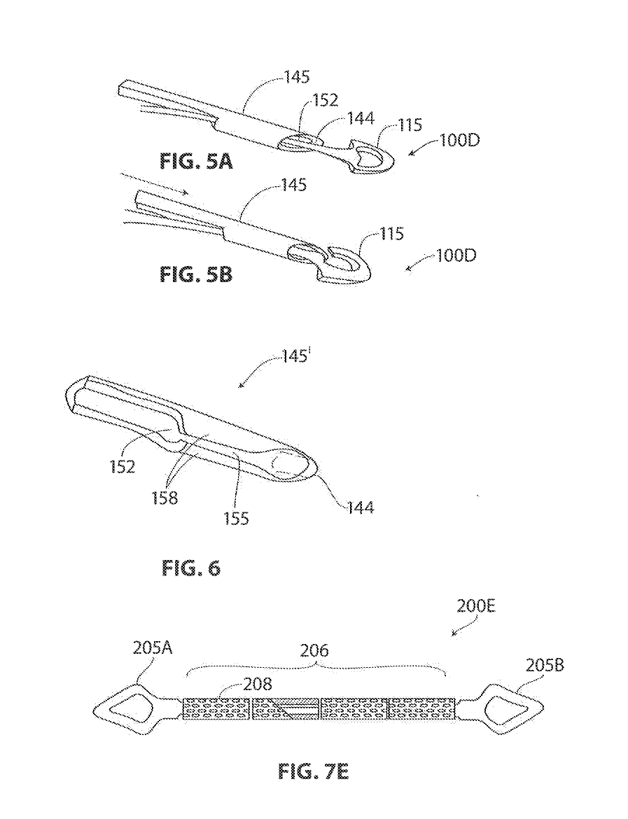

FIG. 4 depicts another embodiment 100D of a revisable implant that is similar to previous embodiments except that at least one end portion 105B includes an indent feature 140 in the proximal-facing aspect of the encircling portion 115 wherein the indent feature 140 is adapted to direct and receive a cutting blade or edge 144 (phantom view) of a cutting tool for cutting the encircling portion of the implant body to allow its removal from the treatment site. As will be described below (FIG. 5B), a cutting tool 145 can be advanced along the medial portion 110 of the implant to sever the end portion, which then will allow the entire implant to be withdrawn from the implant site. In another aspect of the invention, the indent feature 140 in the encircling portion 115 can direct the cutting edge 144 to a reduced cross section portion 148 that will require limited force to cut the polymer element with the cutting edge 144.

FIGS. 5A and 5B illustrate a second component of the kit of a revisable OSA implant system wherein the tool 145 comprises an elongate member with a distal cutting edge 144. One tool embodiment has a passageway 152 extending therethrough for receiving the elongate implant body 100D. In using this tool 145, a first end of the implant body would be freed from tissue or cut and then threaded through the passageway 152. Thereafter, as depicted in FIG. 5B, the tool 145 can be advanced distally while holding the proximal end of the implant to cause the cutting edge 144 to cut across the encircling portion 115. In FIG. 5B, it can be understood how the indent feature 140 and reduced cross section portion 148 (see FIG. 4) direct the cutting edge 144 to easily cut the element to thus release the implant from encircling the tissue plug 112 (cf. FIG. 3B). The tool 145 can be a rigid or semi-rigid member such as a hypotube with a sharpened end. The tool also can be a deflectable, articulatable or deflectable member as in known in the art. In another embodiment, the tool can be a flexible plastic material with a blade insert to provide the cutting edge 144. Referring to FIGS. 5B and 3B, it can be understood that the cut end is flexible and can be pulled from around the tissue plug to extract the implant from the site 120 (see FIG. 3B).

FIG. 6 illustrates another second tool component of system 90 wherein the tool 145′ again comprises an elongate member with a distal cutting edge 144. In one embodiment, the tool end includes a longitudinal gap 155 along a side of passageway 152 to thus allow the tool to be inserted over medial portion 110 of an implant body to then advance and cut the implant as depicted schematically in FIGS. 5A-5B. The tool end as shown in FIG. 6 can comprise a polymer member with flexible elements 158 on either side of gap 155 to allow the device to be inserted over the implant.

FIGS. 7A-7C illustrate other embodiments of implants 200A, 200B and 200C that each have a plurality of the through-openings 206 in various configurations. In these embodiments, the ends are flat or planar with the openings therein. Thus, in use, there will be a plurality of tissue plugs that grow through the openings to secure the implant ends in the tissue site.

FIG. 7D illustrates another embodiment of implants 200D that has a non-planar end 201 with a plurality of through-openings 202. In one embodiment, the ends have a plurality of elements 204 that extend in different radial angles relative to the axis 111 of the implant with each such element 204 having one or more openings therein.

FIG. 7E illustrates an implant body 200E with ends 205A and 205B and medial portion 206 that comprises an axially-stretched and tensioned elastomeric material. The medial portion 206 is releasably and temporarily maintained in the axially-stretched non-repose condition by a biodissolvable magnesium portion indicated at 208. In this embodiment, the magnesium can comprise a thin wall tube, a plurality of thin wall tube segments, or one or more windings or braids of magnesium. The thin-wall magnesium material, or the magnesium filament of a winding or braid, can be very fine and adapted to dissolve and erode with a selected time interval ranging from about 2 weeks to 52 weeks. In another embodiment, the magnesium portion 208 can be disposed in an interior portion of the implant body, in a linear or helical configuration.

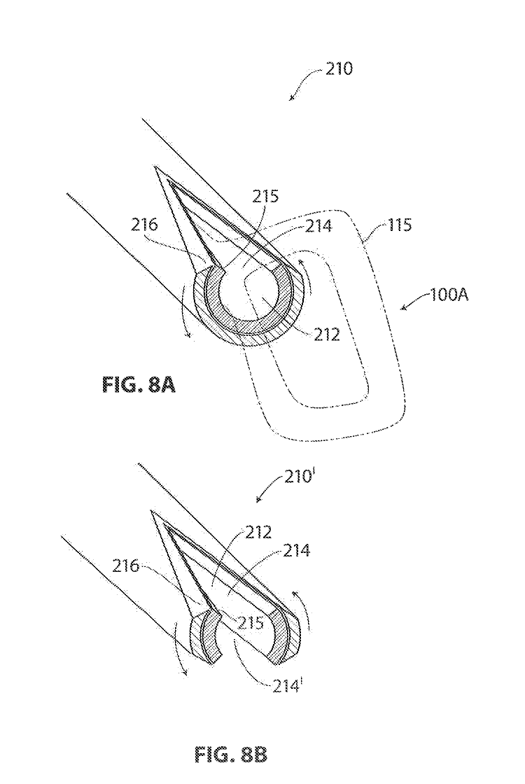

FIG. 8A depicts the working end 210 of an elongated tool that is adapted for cutting an end portion of an implant for its removal, for example an implant of FIG. 3A-3D, 4, or 7A-7D. The tool functions similar to that of FIGS. 5A and 6, wherein the tool has a central bore 212 that receives the elongate medial portion of an implant body. As can be seen in FIG. 8A, the working end 210 includes two concentric hypotubes with a notch 214 therein to push over an end portion 115 of implant 100A of FIG. 3A, for example. The physician can counter-rotate the hypotubes from a proximal handle end wherein blade edges 215 and 216 of the working end function as a scissors mechanism to cut the implant body. Thereafter, the implant can be easily removed from the treatment site. FIG. 8B illustrates another working end 210′ of a similar cutting tool that has opposing notches 214 and 214′ that can receive an implant body portion and blade edges 215 and 216 can be rotated to cut the implant.

FIG. 9 illustrates another embodiment of implant 220 that is similar to any previous embodiment except depicting a difference in surface characteristics of the implant. In one embodiment, the end or encircling portion 225 can have smooth or slightly textured surface features and the medial portion 230 comprises a highly lubricious surface, and in one embodiment comprises an elastomeric material having an ultrahydrophobic surface 232 to allow for slippage of the tissue against the implant during use. Thus, a method of the invention comprises implanting a device in airway-interface tissue, securing first and second implant end portions in the tissue by permitting a tissue growth through at least one opening in end, and allowing an elastomeric portion of the implant to apply retraction forces to alleviate tissue obstruction of the airway wherein an ultrahydrophobic surface of the implant prevents tissue adhesion to said surface. Ultrahydrophobic surfaces can be provided in a biocompatible polymer, as is known in the art.

In another aspect of the invention, referring to FIG. 9, the elongate implant body is configured for implanting in an airway-interface and at least a portion of a body surface has a wetting contact angle greater than 70o, to prevent tissue adhesion and to allow tissue slippage. In another embodiment, at least a portion of a body surface has a wetting contact angle greater than 85o, or greater than 100o.

In another aspect of the invention, still referring to FIG. 9, the elongate implant body is configured for implanting in an airway-interface and at least a portion of a body surface has an adhesive energy of less than 100 dynes/cm, less than 75 dynes/cm or less than 50 dynes/cm.

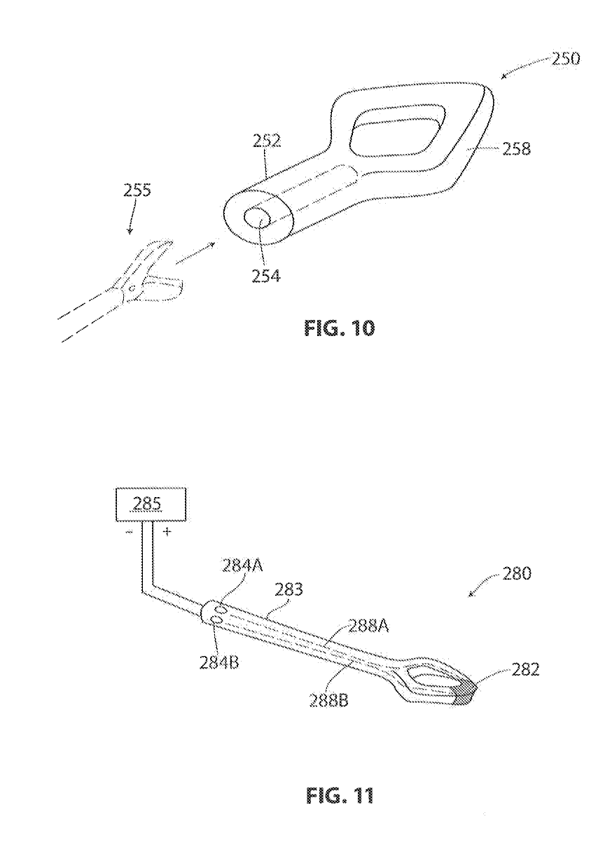

FIG. 10 illustrates another embodiment of revisable OSA implant 250 similar to previous embodiments except the medial portion 252 includes a passageway 254 configured for extending a cutting tool 255 through the passageway for cutting a distal end portion 258 of the implant. The passageway 254 can be accessed by an access opening in the opposing end (not shown) that can be identified by imaging of a marker, visual observation of a marker, by a left-in place guidewire or other suitable means or mechanism. The cutting tool 255 can comprise a scissor member, an extendable blade that is extendable from a blunt-tipped tool, any distal or proximally-facing blade, and/or any type of thermal energy emitter adapted for cutting the implant end 258.

FIG. 11 illustrates another embodiment of revisable OSA implant 280 that has a sacrificial portion indicated at 282 that can be severed or sacrificed by an external stimulus. In one embodiment, a medial portion 283 of the implant includes electrical contacts or extending leads 284A and 284B that can be detachably coupled to an electrical source 285. In FIG. 11, the implant body comprises an elastomeric material as described above and the sacrificial portion 282 comprises a conductively doped polymer portion that acts as a fuse when subject to a very short burst of high voltage RF current. Opposing sides or aspects of the sacrificial portion 282 are coupled to electrical leads 288A and 288B that are embedded or molded into the implant. The use of such doped polymers for a fuse-effect for detachment of endovascular medical implants is disclosed in U.S. Pat. No. 6,458,127 to Truckai et al. and issued Oct. 1, 2002, which is incorporated herein by reference. Similar doped polymers can be used in the revisable OSA implant of FIG. 11.

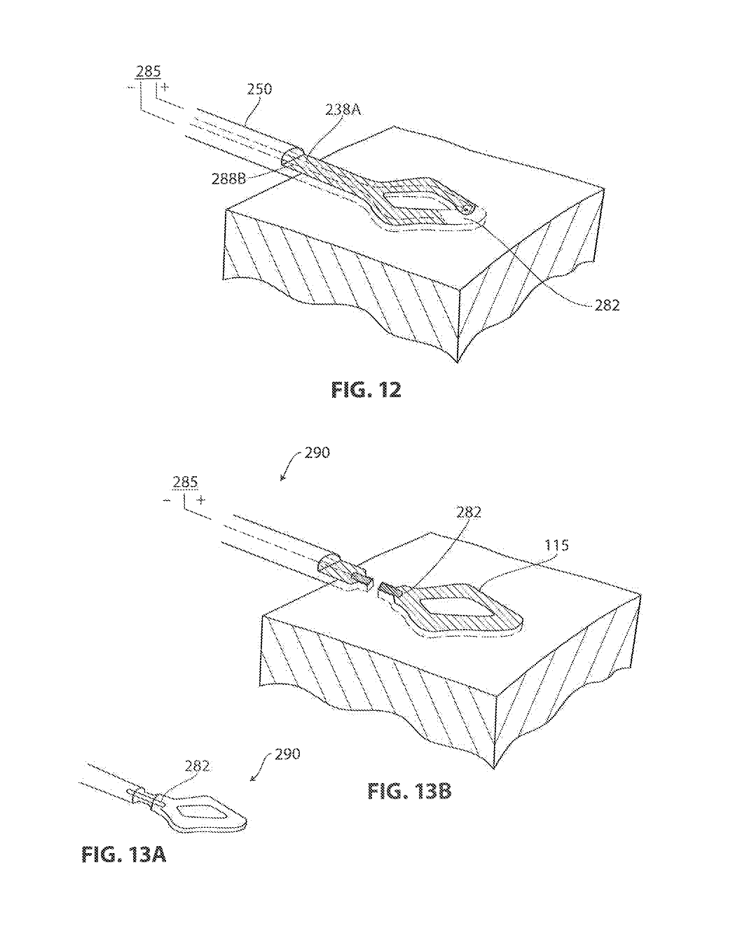

FIG. 12 illustrates a method of using the OSA implant 280 of FIG. 11, and more particularly for revising the treatment. FIG. 12 depicts that an RF current from source 285 has been delivered to melt, sever and sacrifice portion 282 of the implant thus allowing extraction of the implant from around the tissue plug.

FIGS. 13A and 13B illustrate another embodiment of revisable OSA implant 290 that has a sacrificial portion indicated at 282 in a medial portion of the implant that can be actuated and sacrificed by the external stimulus which then leaves the encircling portion 115 of the implant in place. The left-in-place portion of the implant can be used as an anchor for subsequent implants. In one embodiment as in FIGS. 13A-13B, the sacrificial portion 282 can comprise an electrolytic wire that can be sacrificed over a short time interval by direct current as is known in the art. Such electrolytic wire for detachment of embolic coil implants are known in the field of aneurysm implants and treatments.

While FIGS. 11-13B show OSA implants with two forms of sacrificial portions, it should be appreciated that similar implants can have sacrificial portion that are cut, severed or sacrificed by any external stimulus such as RF current, DC current, light energy, inductive heating etc. and fall within the scope of aspects of the invention.

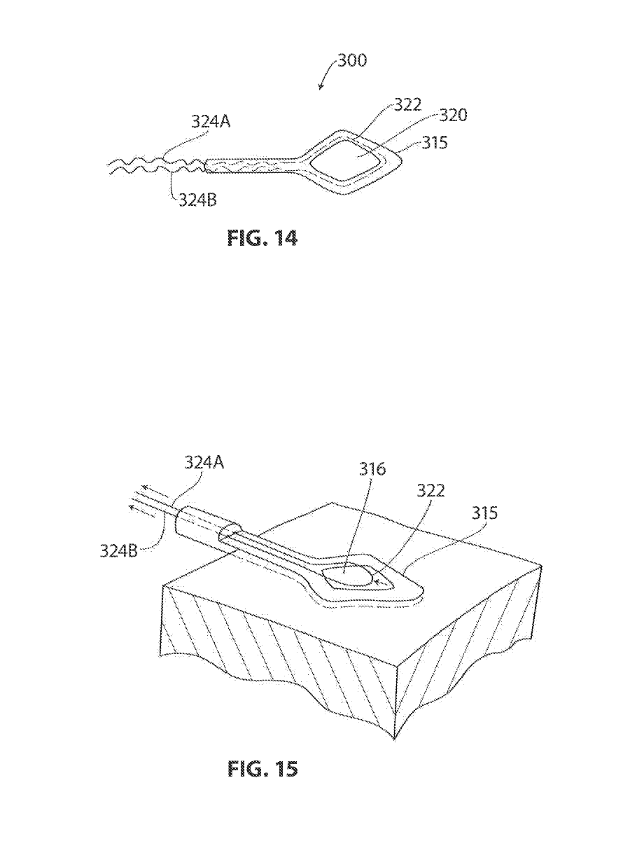

FIGS. 14 and 15 illustrate another embodiment of revisable OSA implant 300 that again includes at least one end with an encircling portion indicated at 315 that encircles a tissue plug 316 that grows through an opening 320. In one embodiment, the implant carries a cut wire 322 that extends in a loop with first and second wire ends 324A and 324B extending through one or more passageways in the implant. The cut wire 322 can be embedded in the surface of the implant surrounding the opening 320. As can be seen in FIG. 15, the looped cut wire 322 can be pulled proximally to cut the tissue plug 316 which then will free the implant from its attachment. In FIG. 14, it can be seen that the cut wire ends 324A and 324B can have a serpentine configuration in the medial portion of the implant so as to not interfere with the tensioning and relaxation of the elastomeric medial implant portion during its use. When the cut wire is accessed and pulled relative to the implant 300, the tissue plug 316 can be cut. It should be appreciated that other tools (not shown) may be used to stabilize the implant when actuating the cut wire as in FIG. 15. The cut wire 322 can be any form of fine wire, or abrasive wire or a resistively heated wire coupled to an electrical source (not shown).

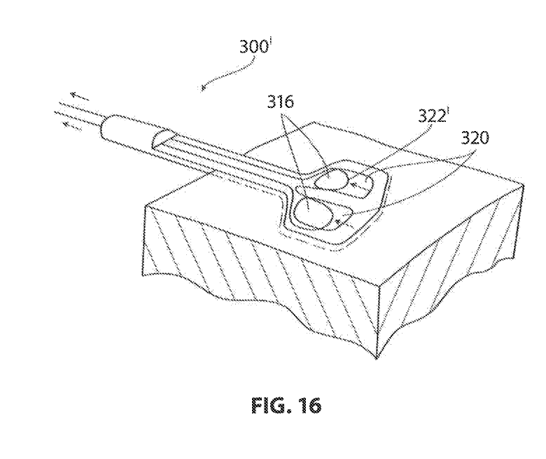

FIG. 16 depicts another revisable OSA implant 300′ that is similar to that of FIGS. 14-15 with the cut wire 322′ configured to cut a plurality of tissue plugs 316 that have grown through openings 320 within an encircling end portion of the implant body.

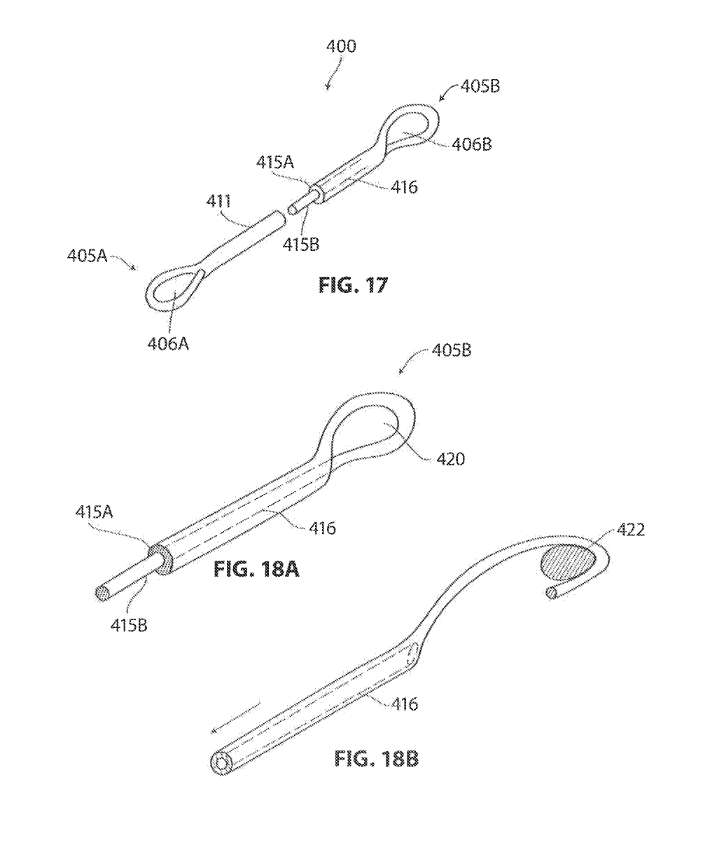

FIG. 17 depicts another OSA implant 400 that is adapted for revision as previous implants and system wherein the elongate device or implant body has first and second end portions 405A and 405B with through-openings 406A and 406B therein. The medial portion 411 of implant body 400 extends about an axis and comprises a biocompatible elastomeric material such as a silicone. In this embodiment, the medial portion comprises first and second extending portions 415A and 415B wherein one such portion can be nested in a passageway 416 of the other portion and then form proximal and distal loops or encircling end portions that define openings 406A and 406B for receiving tissue plugs therein. As can be understood from FIGS. 17 and 18A, both the extending portions 415A and 415B comprise an elastomeric material and thus combine to provide the desired retraction forces of the OSA implant.

Referring to FIGS. 18A and 18B, it can be seen that if the second extending portion 415B is cut in a medial or proximal aspect of the implant, or if both the first and second extending portions 415A and 415B are cut in a proximal or medial aspect, then a proximal aspect of the first or outer extending portion 415A can be pulled in the proximal direction and the cut second extending portion 415B then will snake out of the path around the tissue plug 422. Thus, the implant can be cut in a proximal or medial aspect and can be withdrawn from the treatment site from a remote access location.

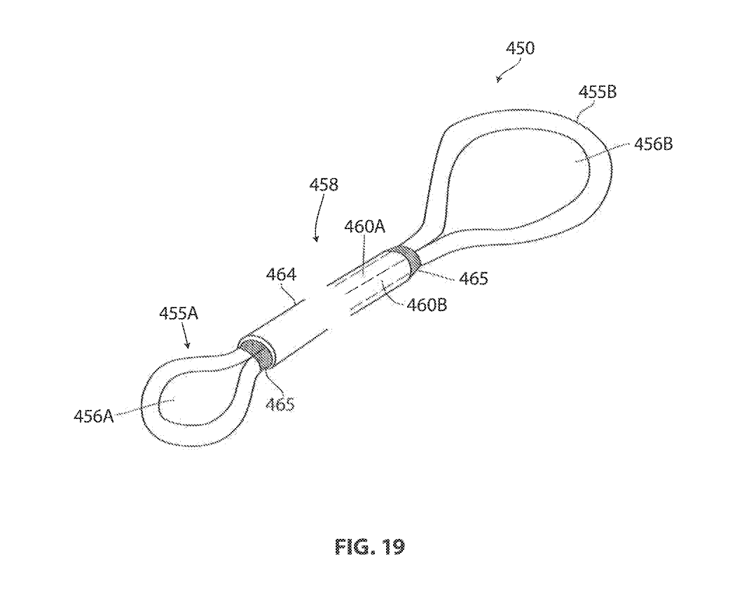

FIG. 19 depicts another OSA implant 450 that is adapted for a revision procedure and comprises an elongate implant body with first and second end portions 455A and 455B with through-openings 456A and 456B therein. This embodiment is similar to that of FIG. 17 in that medial portion 458 includes extending portions 460A and 460B comprise an elastomeric material that combine to provide the desired retraction forces of the OSA implant. The extending portions 460A and 460B are carried in a thin elastomeric sleeve 464 that has tear-away portions 465 about its ends to prevent tissue ingrowth into the passageway in the sleeve. It can be understood that by cutting the medial portion of the implant, and then pulling on an end of an extending portions 460A or 460B will cause the other free end of the implant to snake around the tissue plug similar to the method depicted in FIG. 18B. Both ends of the implant can be removed from the treatment site by this method.

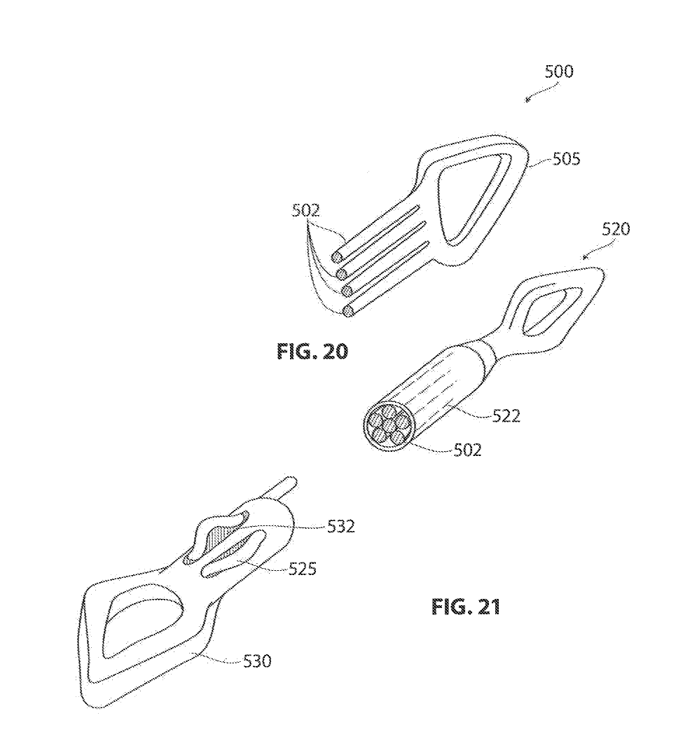

FIG. 20 depicts another revisable OSA implant 500 that is adapted for minimally invasive in-situ post-implant adjustment of retraction forces applied by the implant. In this embodiment, the implant is configured for a downward adjustment of retraction forces applied by the OSA implant. In FIG. 20, it can be seen that the elongate implant body has a plurality of extending elements 502 coupled to end portion 505, wherein the elements 502 can be individually cut to reduce the applied retraction forces of the implant. The number of extending elements 502 can range from 2 to 20 or more.

FIG. 21 depicts a revisable OSA implant 520 that functions as the previous embodiment except that the plurality of extending elements 502 are housed in thin-wall elastomeric sleeve 522. Further, an axial portion 525 of each extension element 502 protrudes outward from sleeve 522, or an end portion 530 of the implant, to allow such a portion to be cut. Again, any form of cutting tool can be used for minimally invasive access to cut an elastomeric element to titrate retraction forces in a downward direction.

### C. In-Situ Adjustable Force OSA Implants

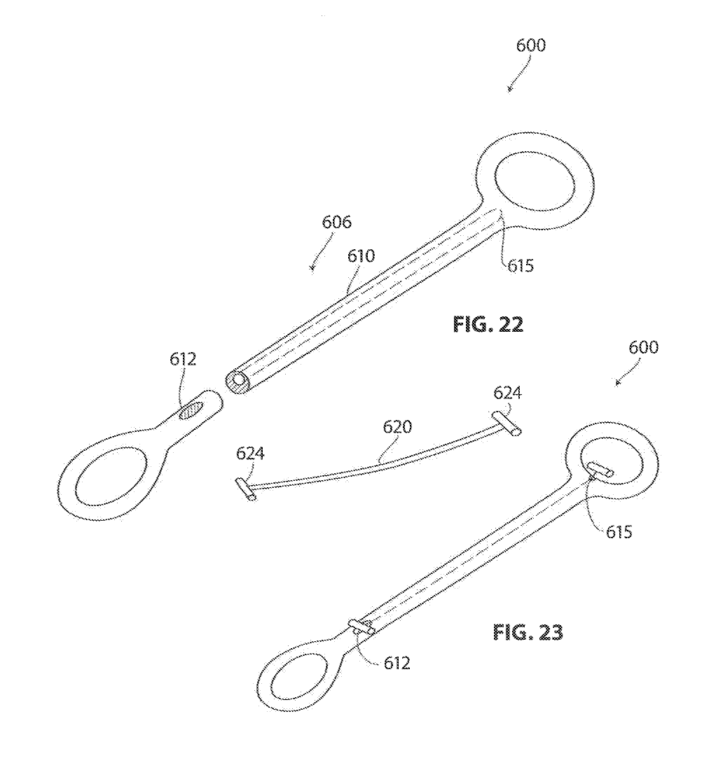

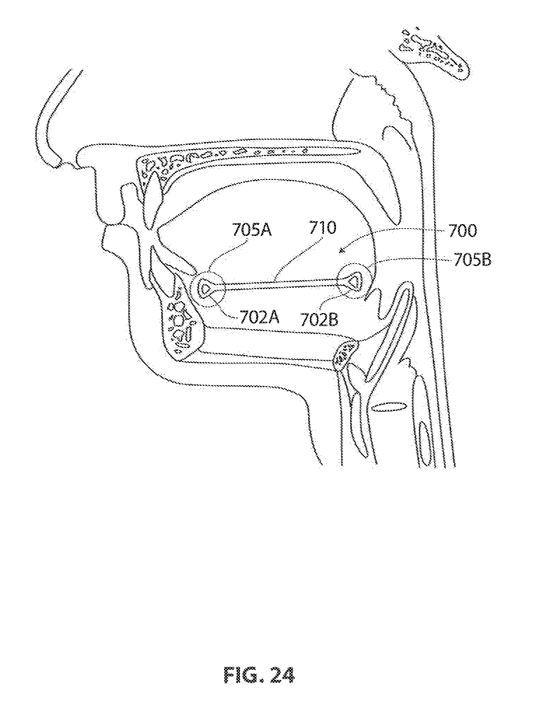

Another type of OSA implant includes means for in-situ adjustment of force applied by the implant after implantation in the treatment site. Such an adjustment can increase or decrease the applied forces applied to the treatment site by the implant. Such adjustment of forces applied by the implant typically may be performed upon specific event, such as periodic evaluations of the treatment. The adjustment also can be done at a pre-determined schedule, based on an algorithm, or can be random. In one example, the patient may gain or lose weight which could result in a need for adjusting the forces applied by the implant. Other influences can be a worsening of the patient's condition, the aging of the patient, local tissue remodeling around the implant, age of the implant or degradation of material properties of the implant. In another embodiment described below, an implant system can be provided that is easily adjustable in-situ between first and second conditions on a repetitive basis, for example, that can be adjusted for sleep interval and for awake intervals on a daily basis. Such an adjustable embodiment can thus deliver tissue-retraction forces only when needed during sleep. One advantage of such an embodiment would be to allow the tissue of the treatment site to be free from implant-generated retraction forces during awake intervals to prevent or greatly limit the potential of tissue remodeling due to a continuous application of such retraction force. FIG. 22 depicts an OSA implant 600 that is adapted for in-situ post-implant adjustment of retraction forces applied to targeted tissue. In one method, assume that it is desirable to increase the applied retraction forces over time due to tissue remodeling wherein greater retraction forces are desired. In FIG. 22, the elongated implant body has a medial portion 606 that includes an interior channel 610 that extends from an accessible first end 612 to a remote end 615. Each end 612 and 615 can include a silicone membrane to prevent tissue ingrowth but will allow a needle to be inserted therethrough. The channel ends 612 and 615 can be disposed in more rigid end portions of the implant, wherein the medial portion of the implant body comprises an elastomer to provide the desired retraction forces. In one embodiment, the channel 610 is dimensioned to collapse or flatten but can also accommodate the insertion of at least one additional elastomeric element indicated at 620. It can be understood from FIG. 23 that an elastomeric element 620 with end-toggles 624 be inserted in a bore of a flexible needle member (not shown) and inserted through the channel in the implant so that the toggles are released to deploy the element 620 in a tensioned position to thereby add to the retraction forces applied to tissue collectively with the medial portion 606 of the implant 600. In a similar manner, an end of the implant can be clipped to reduce the applied retraction forces as in the system and method depicted in FIGS. 20 and 21.

Thus, in general, the system and implants of FIGS. 20-23 corresponding to aspects of the invention comprise an elongate implant sized and shaped to conform to an airway-interface tissue site in a manner compatible with normal physiological function of the site, a medial portion of the implant comprising an elastomeric material configured to apply retraction forces to the site, and adjustment means for in situ adjustment of retraction forces applied by the implant.

### D. OSA Implants for Applying Non-Aligned Displacement Forces

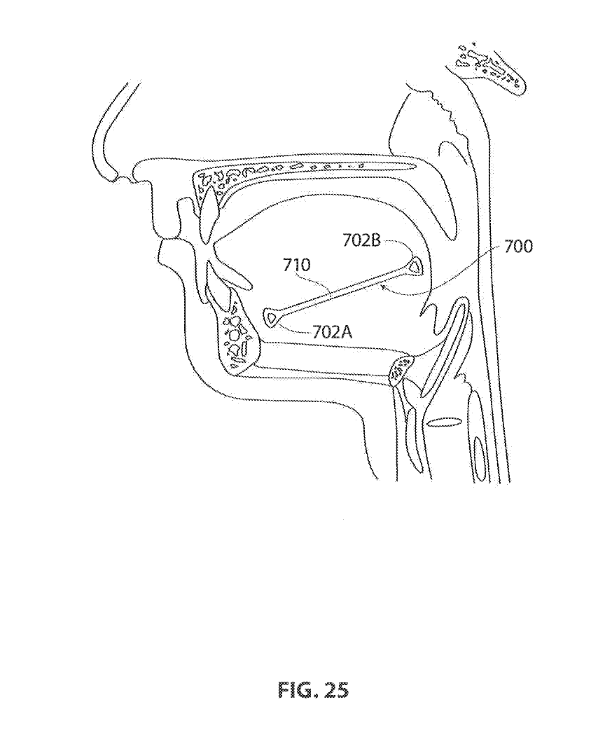

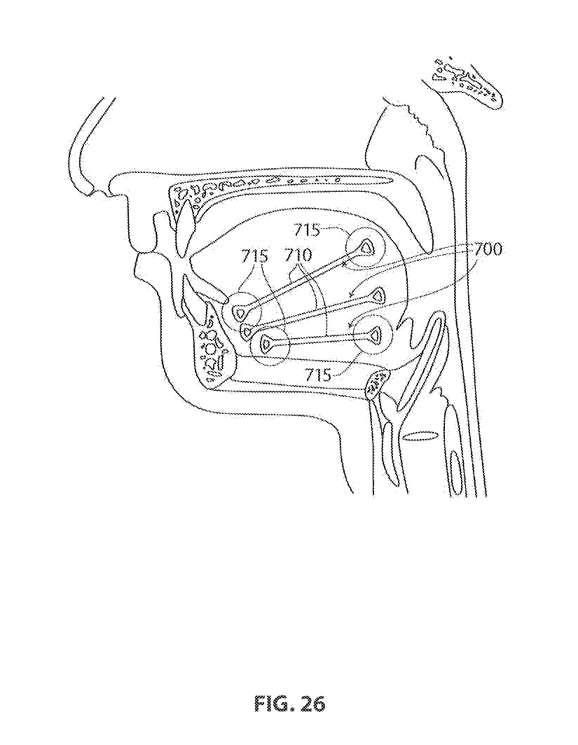

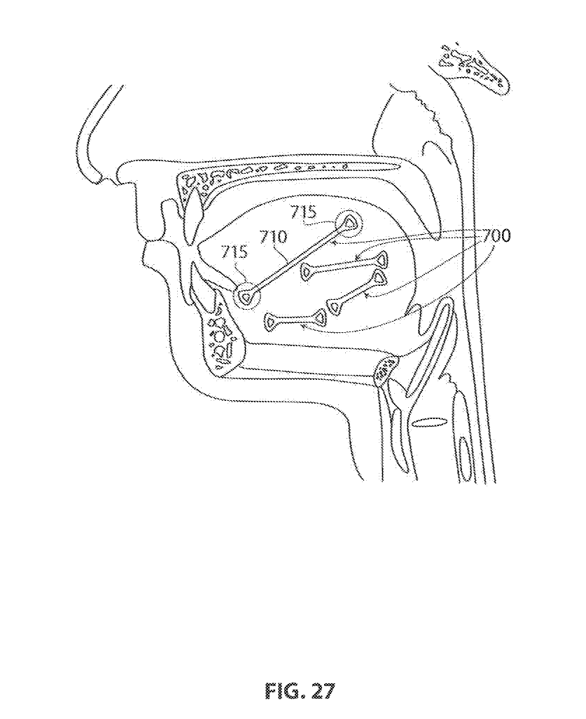

Another aspect of the invention can be described with reference to FIG. 24-27, wherein a resilient implant (or implants) can be positioned in airway-interface tissue to apply tensile forces or displacement forces in at least two non-aligned directions or vectors. In a typical embodiment depicted in FIGS. 24-25, an implant 700 corresponding to aspects of the invention can form a linear structure wherein two anchor ends 702a and 702b form anchor points or regions 705a and 705b in the tissue. Such points 705a and 705b are connected by a straight or substantially straight elastic portion 710 or spring element of the implant such that said elastic portion or spring element applies a tensile force and/or a tensile displacement between said anchor points 705a and 705b. In the embodiment of FIG. 24, the implant 700 acts to apply forces and/or displacements between the said anchor points 705a and 705b to displace and/or apply forces to the patient's tongue, but it should be appreciated that an appropriately dimensioned implant can also or instead be introduced into the soft palate or pharyngeal structures adjacent to the patient's airway. FIG. 25 illustrates the implant 700 can have various orientations in the tissue. Now turning to FIGS. 26-27, it can be seen that a plurality of substantially linear elastic implants 700 similar to that of FIGS. 24-25 can thus provide a plurality of tissue anchor points 715 wherein the elastic or spring portion 710 of the implants function in such a manner to provide tensile or displacement forces to achieve the desired clinical effects. Testing in animal models has indicated that forces applied to the subject's tongue by two implants in two different directions may improve implant performance when compared with unidirectional application of forces from a single implant.

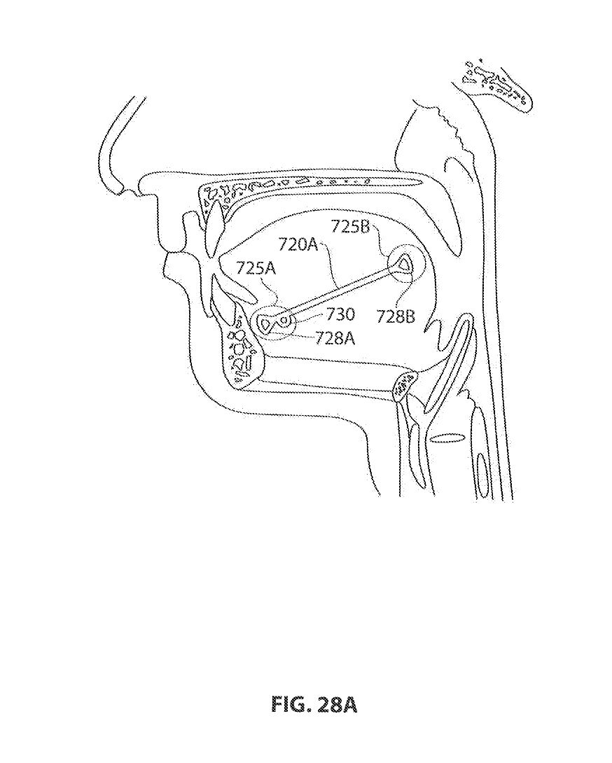

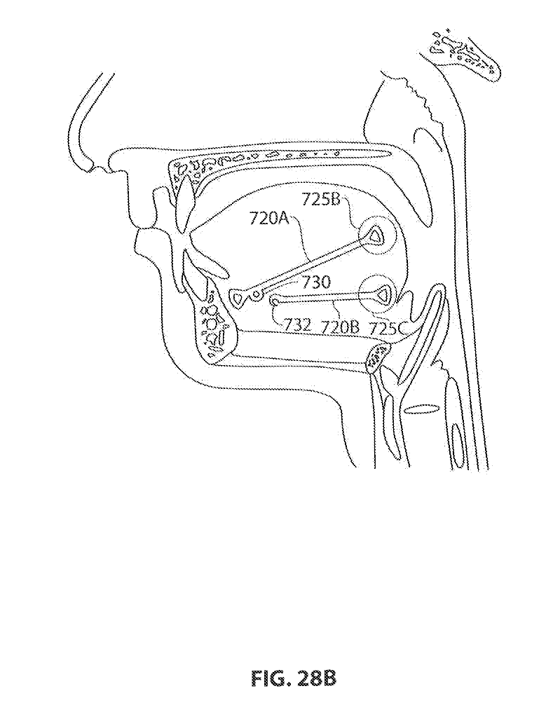

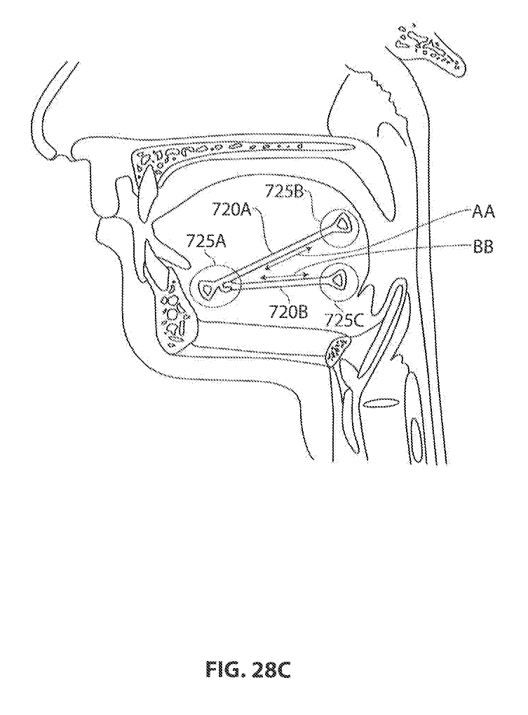

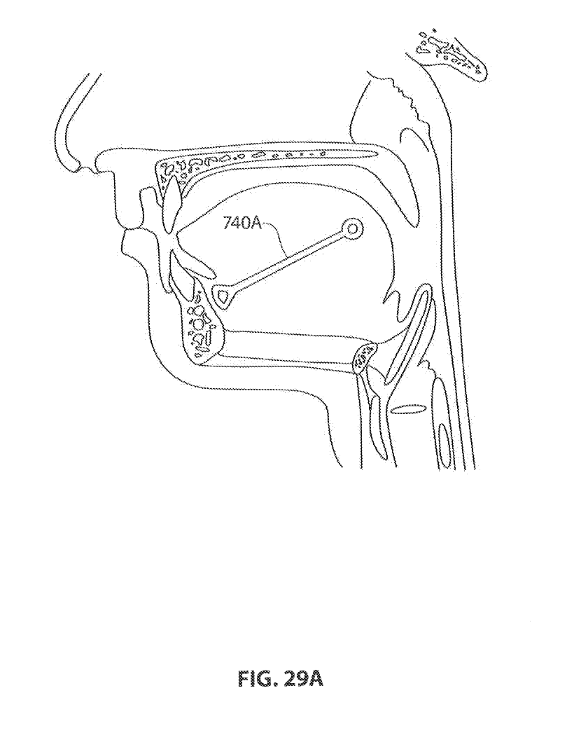

FIGS. 28A-28C schematically illustrate another embodiment of implant system according to aspects of the invention that comprises first and second elastic elements 720A and 720B that provide three anchor points in tissue indicated at 725a, 725b and 725c. FIG. 28A depicts the implantation of the first elastic element 720A which has anchoring ends 728a and 728b as described above, wherein at least one end is configured with an attachment element such as a loop 730 that is connectable with a hook element 732 of a second elastic element 720B. Thus, FIGS. 28A and 28B depict the steps of implanting the elastic elements wherein elastic element 720A is initially implanted in its desired location. Then, FIG. 28B depicts elastic element 720B being positioned in its desired location such that the hook 732 is adjacent to loop 730 of the elastic element 720A. FIG. 28C then depicts the loop 730 and hook 732 be connected in such a manner to produce a fixed-link implant structure which thus applies forces in two non-aligned vectors AA and BB. It can be understood that the implants can be implanted in sequence and then coupled in situ to form a V-shaped implant system. It should be appreciated that the implant structure of FIGS. 28A-28C can have components such as elastic or spring elements that can be connected prior to, during, or following implantation by means of adhesives, connectors, snap-fit features, hooks and loops, clamps, ratchets, keyed fittings, etc., or by means of separate attachment, such as sutures, junctions, clamps, or other connection means. In another embodiment, two end portions of separate implant bodies can be disposed proximate to one another, and the body's fibrotic response or wound healing response can cause a connection of the two implant ends.

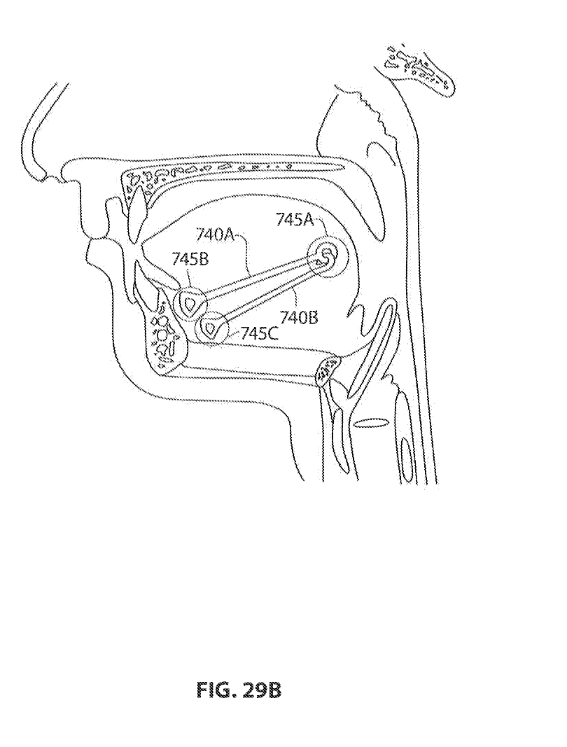

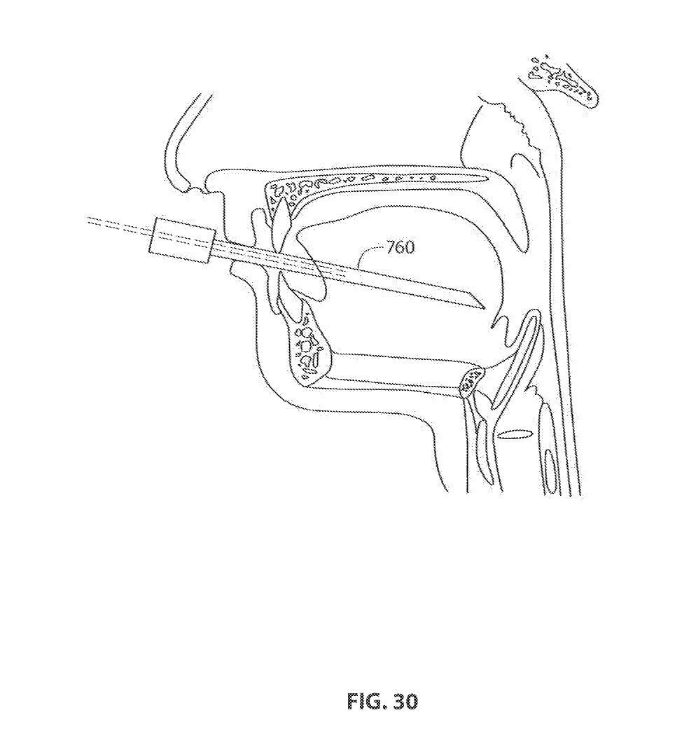

FIGS. 29A-29B schematically illustrate another embodiment of implant system comprising first and second elastic elements 740A and 740B in a different orientation in a patient's tongue. Each implant has an elastic medial section as described above. The implant system again provides three anchor points 745a-745c as shown in FIG. 29B, wherein the first implant can be fixedly attached to the second implant by loop and hook features or other similar means. As described previously, the implants can be implanted in sequence and then coupled in situ to form the V-shaped implant system. In some embodiments, the angle between the legs of V-shaped implant can range from about 10o to 170o, depending on the implant site. The lengths of the legs of the V-shaped implant can vary, as well as the forces applied by each leg of the V-shaped implant.

In general, when the implants of the disclosure as described above are implanted in the tongue and/or the palate of the patient, the positioning of the implants will affect the location and direction of the applied forces and the displacements of the surrounding tissues. The implants may be placed in various locations to achieve the desired clinical effects, and may be specifically tailored to an individual patient based on the nature and details of each patient's OSA, including their specific anatomy and physiology. For example, if a patient suffers obstructions associated with the lower posterior region of the tongue impinging on the posterior pharyngeal wall, then an implantation location that places one end of a linear implant lower in the tongue may be appropriate (see FIG. 24). In another example, if the patient suffers obstructions associated with the upper posterior region of the tongue impinging on the posterior pharyngeal wall, then an implantation location that places one end of a linear implant higher in the tongue may be more appropriate (see FIG. 25). In a similar manner, the implants of the disclosure may be placed in various locations within the tongue and soft palate, utilizing one or more implants, to address the specific needs of the patient and to achieve the desired clinical effects.

In general, a method according to aspects of the invention for treating an airway disorder comprises implanting at least one elastic implant in airway-interface tissue wherein the at least one implant in configured to apply tensile forces to the tissue in at least two non-aligned directions or vectors. The non-aligned vectors thus describe the linearly-directed forces applied to tissue by substantially linear, elongated implants disposed in the tissue, such as vectors AA and BB in FIG. 28C.

In one aspect of the method, the linearly-directed forces can be applied to tissue in the non-aligned vectors by a single implant configured with first and second body portions that extend in between different anchoring sites. In another aspect of the method, at least first and second implants can be implanted to apply such forces in at least first and second non-aligned vectors. In any implant embodiment, the elongated elastic body portions can cooperate with bioerodible materials that temporarily maintain the implant in an extended position as described above. Further, as described previously, the targeted airway-interface tissue which receives the implant can comprise the patient's tongue, soft palate and/or pharyngeal tissue.

### E. Implant Force and/or Movement Parameters

Implant Force Threshold. The implants of the disclosure may apply forces and displacements to anatomical structures within the patient's airway, including the tongue and soft palate, to treat obstructive sleep apnea (OSA) by repositioning and/or applying forces to said anatomical structures in such a manner as to provide an open airway during normal breathing. The forces applied by said implants to said anatomical structures are large enough to sufficiently to move, or displace, said structure so as to provide a clear airway when the patient is asleep, but are not so large as to damage the surrounding tissue, damage the implant, prevent proper airway function, or prevent proper tongue function such as normal speech and swallowing.

When the one or more implants of the disclosure are employed within the patient's tongue to prevent airway occlusion associated with OSA when said patient is asleep and fully relaxed, said implant(s) provide sufficient force to allow the airway to open during normal breathing. The force necessary to open said airway during normal breathing may be a force less than the weight of the tongue itself, as normal breathing provides an internal pressure that acts to help open the airway. The minimum force supplied by said implant(s) to allow the airway to open during normal breathing is referred to as the minimum threshold force for therapeutic benefit. This minimum threshold force for one or more implants within or adjacent to the tongue is 0.5 Newtons in some embodiments, the minimum threshold force is 1.5 Newtons in other embodiments, and the minimum threshold force is 3.5 Newtons in still other embodiments.

When one or more implants of the disclosure are employed within the patient's soft palate to prevent airway occlusion associated with OSA when said patient is asleep and fully relaxed, said implant(s) provide sufficient force to deflect the soft palate away from the back wall of said patient's throat thus providing an open airway. As with the tongue, the force necessary to open said airway during normal breathing may be a force less than the weight of the soft palate itself, as normal breathing provides an internal pressure that acts to help open the airway. The minimum force supplied by said implant(s) to allow the airway to open during normal breathing is referred to as the minimum threshold force for therapeutic benefit. This minimum threshold force for one or a more implants within or adjacent to the soft palate is 0.2 Newtons in some embodiments, the minimum threshold force is 0.5 Newtons in other embodiments, and the minimum threshold force is 1.0 Newtons in still other embodiments.

Implant Motion Threshold The implants of the disclosure apply forces and displacements to anatomical structures within the patient's airway, including the tongue and soft palate, to prevent obstructive sleep apnea (OSA) by repositioning said anatomical structures. The displacements applied by said implants to said anatomical structures are large enough to sufficiently move, or displace, said structures so as to provide a clear airway when the patient is asleep, but are not so large as to cause adverse side effects. Said side effects may include limited tongue or soft palate function resulting in adverse effects on speech and/or swallowing, difficulty breathing, unwanted remodeling of tissues over time, damage to soft or hard tissues, and causing said soft structures, like the tongue or soft palate, to interfere with other anatomical structures or to cause other unwanted effects.

When implanted within the tongue, the implants of the disclosure provide forces and displacements to the tongue to allow the patient's airway to remain open during normal breathing when the patient is asleep and fully relaxed. The maximum displacement of the tongue that does not result in undesired side effects, as mentioned above, is referred to as the maximum threshold displacement for therapeutic benefit. This maximum threshold displacement for one or a more implants within or adjacent to the tongue is between about 0.5 mm and about 20 mm in some embodiments, between about 1.0 mm and about 15 mm in other embodiments, and between about 1.0 mm and about 10.0 mm in still other embodiments.

When implanted within the soft palate, the implants of the disclosure may provide forces and displacements to the soft palate to allow the patient's airway to remain open during normal breathing when the patient is asleep and fully relaxed. The maximum displacement of the soft palate that does not result in undesired side effects, as mentioned above, is referred to as the maximum threshold displacement for therapeutic benefit. This maximum threshold displacement for one or a more implants within or adjacent to the soft palate is from 0.5 mm to 5.0 mm.

When implanted in the tongue, the implants of the disclosure may provide an effective therapeutic window of operation bounded by a minimum threshold force required to prevent the tongue from obstructing the airway during normal breathing when the patient is asleep and relaxed, and by a maximum displacement threshold above which the implant(s) adversely affects normal airway and tongue function including speech, swallowing, breathing, etc. This effective therapeutic window is identified based on the forces and displacements described above.

When implanted in the soft palate, the implants of the disclosure may provide an effective therapeutic window of operation bounded by a minimum threshold of force required to prevent the soft palate from obstructing the airway when the patient is asleep and relaxed, and by a maximum displacement threshold above which the implant(s) adversely affects normal airway or mouth function including speech, swallowing, breathing, etc. This effective therapeutic window is identified based on the forces and displacements described above.

Implant Force/Motion Directions within the Tongue. When the one or more implants of the disclosure are employed within the patient's tongue to prevent airway occlusion when said patient is asleep and fully relaxed, said implant(s) provide sufficient force to open the airway during normal breathing. One or more implants may be employed to apply the desired forces and deflections to the patient's tongue. Said implants may be employed in one or more locations within or adjacent to the tongue, they may be anchored in one or more locations within or adjacent to the tongue, and they may apply forces and/or deflections in one or more directions and between two or more locations within or adjacent to the tongue.

Said implants may be employed in such a manner as to relieve obstructions in the airway caused by the tongue resulting in OSA. Generally, this includes displacing the posterior region of the tongue and/or providing forces on the posterior region of the tongue that pull said posterior region in the anterior direction, away from the posterior pharynx wall, resulting in keeping the opening of the airway the airway from closing such that normal breathing can be maintained. Said forces and/or displacements may act to affect the entire posterior region of the tongue, a very specific location in the posterior region of the tongue, a linear area of affect in the posterior region of the tongue (i.e., a linear area that runs cranially and caudally so as to create a channel through which the airway remains patent), or any combination of the above.

In one example exemplary embodiment, a single implant is employed to apply a force to the posterior region of the tongue in an approximately horizontal anterior direction as viewed in a patient standing straight up with their head facing forward (FIG. 24). In another exemplary embodiment, a single implant is employed to apply a force to the posterior region of the tongue at an inclined angle to the horizontal, and in the anterior direction as viewed in a patient standing straight up with their head facing forward (FIG. 25).

In another embodiment of the invention, more than one implant can be used to apply the appropriate therapeutic force(s). As shown in FIG. 26, three implants are employed within the tongue to apply forces to the posterior region of the tongue in such a manner as to advantageously create a longitudinal open region between said tongue and the posterior pharyngeal wall, running in the direction of air motion during normal breathing. The three implants in this embodiment are acting in different directions to create the desired net distribution of forces and displacements on the tongue (FIG. 26). In another embodiment of the invention, four implants are employed within the tongue to apply forces distributed throughout the tongue, with the implants acting in different directions to create the desired net distribution of forces and displacements on the tongue (FIG. 27).

When more than one implant is used, the set of implants may all lie in any orientation with regard to each other and the surrounding anatomical structures, including in a linear arrangement, a parallel arrangement, a planar array (including but not limited to a triangulated structure), a three-dimensional array, or any combination of these arrangements. The implants may be joined together in any multi-linear, non-linear, or multiply-linearly segmented manner. One example is described above in FIGS. 28A-28C, wherein two linear elastic or spring elements 720A and 720B are connected to provide a common anchor point 725a in tissue at one end of each of the two said linear elements, respectively. The other ends of the first and second linear elements provide additional anchor points 725b and 725c in the tissue. In this manner, anchor points 725b and 725c are pulled in the direction of the common anchor 725a so as to provide a bi-linear implant structure. By extension, and in this manner, complex multi-linear structures or networks of linear elements may be constructed to achieve the desired clinical effects. Similarly, two or more implants comprising multi-linear components may be employed in conjunction to achieve the desired clinical effects. Alternately, the elastic or spring elements may be fabricated in such a fashion as to produce a joined, jointed, or linked structure during the manufacturing process.

FIGS. 35A-35B illustrate another method of treating an airway disorder which comprises implanting two elongated implants 1200A and 1200B similar to those described above in a patient's tongue 1204 in a non-parallel orientation. In the side view of FIG. 35A, it can be seen that the anterior ends 1206a and 1206b of the implants 1200A and 1200B, respectively are anchored proximate the patient's mandible 1208. The anterior ends can be fastened directly to the mandible or implanted in tissue adjacent the mandible. In another variation, the anterior ends can be coupled to each other or coupled to one another and slidably coupled to an anchor in the mandible.

In FIGS. 35A and 35B, it can be seen that the posterior ends 1212a and 1212b of implants 1200A and 1200B, respectively, are positioned in a posterior region of the base 1214 of the patient's tongue. As can be seen in FIGS. 35A-35B, one variation of a method corresponding to the invention comprises implanting the two elongated implants in the tongue wherein the orientations of the implant axes are non-parallel. In particular, the posterior ends 1212a and 1212b of implants 1200A and 1200B are spaced apart vertically by a selected dimension V which can be at least about 0.25 cm, at least about 0.50 cm, at least about 1 cm or at least about 1.5 cm. In one variation, the spacing indicated at V in FIG. 35A can be between about 1 cm to and about 1.5 cm. Referring to FIG. 35B, the implants 1200A and 1200B can be on opposing sides of the mid-line 1220 of the tongue with the anterior implant ends 1216a and 1216b close to the mid-line 1220 and the posterior ends 1212a and 1212b spaced transversely from the mid-line 1220 a distance T that can range from 0 to 1 cm. The implants can lie on opposing sides of the median longitudinal raphe 1222 of the tongue. For example, the implants may be on opposite sides of a sagittal plane of the patient, and in particular may be on opposite sides of a mid-sagittal plane (a longitudinal plane that divides the body into left and right sections). In this variation, it has been found that restraint provided by the implants over a vertical region of the base of the tongue can assist in preventing airway obstruction. In all other respects, the implants depicted in FIGS. 35A-35B can be the same or similar to the implants described earlier in this disclosure, with all, some, or none of the features. For example, the implants may have an expanded configuration and a contracted configuration and may be held in the expanded configuration, such as by a bioerodible portion.

In general, a method includes implanting first and second elongated implants in a patient's tongue, wherein each implant has an anterior end in an anterior location and a posterior end in a posterior location in the patient's tongue, and wherein the posterior end locations are asymmetric relative to a transverse plane. Further, each implant may be asymmetric relative to the mid-line of the tongue.

A method of treating an airway disorder or otherwise treating airway, mouth, nasal, or throat tissue may include implanting at least first and second elongated implants in a tongue of a patient, wherein each of the first and second implants is configured to have a first, expanded configuration and a second, contracted configuration, wherein implanting comprises implanting the first and second implants having their first, expanded configurations, and wherein each implant has an anterior end in an anterior location and a posterior end in a posterior location in the patient's tongue and the posterior end locations are different vertical distances from a transverse plane of a patient. The implants may have a bioerodible portion and an elastomeric portion, and the method may include holding the respective elastomeric portion of each implant in the first expanded configuration with the respective bioerodible portion of the implant.

Another method of treating an airway disorder comprises implanting at least first and second elongated implants in a patient's tongue wherein each implant has an axis and wherein the first axis of 1228a of the first implant 1200A and the second axis 1228b of the second implant 1200B are non-parallel relative to the mid-line 1220 of the tongue (FIG. 35B). Further, the first axis 1228a and the second axis 1228b of the implants may be asymmetric relative to a transverse plane (FIG. 35A).

Another method of treating an airway disorder or otherwise treating airway, mouth, nasal, or throat tissue may include implanting at least first and second elongated implants in a tongue of a patient, wherein each implant is configured to have a first, expanded configuration and a second, contracted configuration and implanting comprises implanting the first and second implants in their first expanded configurations, and wherein each implant has an axis and wherein the axis of the first implant and the axis of the second implant are oblique relative to at least one of a midline plane of the tongue and a transverse plane of the tongue. In a particular embodiment, the axis of the first implant and the axis of the second implant may be oblique relative to both the midline plane of the tongue and the transverse plane of the patient.

FIG. 36 illustrates another implant configuration and method for treating an airway disorder which comprises implanting a plurality of axially-extending implants in a patient's tongue wherein the implants are disposed on one side of the patient's mid-line. For example, in FIG. 36, implants 1230A and 1230B are disposed on one side of the mid-line 1220 of the tongue.

Implant Force/Motion Directions within the Soft Palate. When the one or more implants of the disclosure are employed within the patient's soft palate to prevent airway occlusion when said patient is asleep and fully relaxed, said implant(s) provide sufficient force to open the airway during normal breathing. One or more implants may be employed to apply the desired forces and deflections to the patient's soft palate. Said implants may be employed in one or more locations within or adjacent to the soft palate, they may be anchored in one or more locations within or adjacent to the soft palate, and they may apply forces and/or deflections in one or more directions and between two or more locations within or adjacent to the soft palate.

Said implants may be employed in such a manner as to relieve or prevent obstructions in the airway caused by the soft palate resulting in OSA. Generally, this includes displacing the posterior region of the soft palate and/or providing forces on the posterior region of the soft palate that pull said posterior region in the anterior direction away from the posterior wall of the pharynx resulting in the opening of the airway during normal breathing. More specifically, said implants within said soft palate tend to cause a curvature of the soft palate in the downward and anterior direction to affect said opening of said airway. Said forces and/or displacements may act to affect the entire posterior region of the soft palate, a very specific location in the posterior region of the soft palate, a linear area of affect in the posterior region of the soft palate, or any combination of the above.

In one exemplary embodiment, a single implant is employed to apply a force to the posterior region of the soft palate resulting in a curvature of said soft palate that displaces said soft palate away from the pharynx wall. In another embodiment of the invention, two implants are employed within the soft palate at differing angles and in different locations to apply forces and displacements to the soft palate resulting in a curvature of said soft palate that displaces said soft palate away from the pharynx wall.

FIGS. 37-41 illustrate variations of methods for treating an obstructive airway disorder relating to implanting at least first and second elongated implants in a patient's soft palate 1232. The implants can be of the types described above which include anterior and posterior anchoring ends and an elongated resilient medial region. FIG. 37 illustrates implants 1240A and 1240B which are implanted in the soft palate 1232 with each implant axis extending between the anchoring ends being symmetric and parallel relative to the patient's mid-line 1220. In general, the palate implants have a length of about 2.5 cm to 3.0 cm.

A method of treating an obstructive airway disorder or otherwise treating airway, mouth, nasal, or throat tissue may include implanting at least first and second elongated implants in a patient's soft palate, each implant having anchoring ends and configured to have a first, expanded configuration and a second, contracted configuration, and implanting comprises implanting the implants each having a first, expanded configuration, each implant further having an axis extending between its anchoring ends, wherein the axis of the first implant and the axis of the second implant are symmetric relative to a mid-line of the patient.

FIG. 38 illustrates another variation in which implants 1242A and 1242B are implanted in the soft palate 1232 with the implant axes being symmetric relative to the mid-line 1220 but converging in the posterior direction in the soft palate.

The variation of FIG. 39 is similar to that of FIG. 38 except the implants 1244A and 1244B in the soft palate 1232 have axes that are symmetric relative to the mid-line 1220 but diverge in the posterior direction in the soft palate.

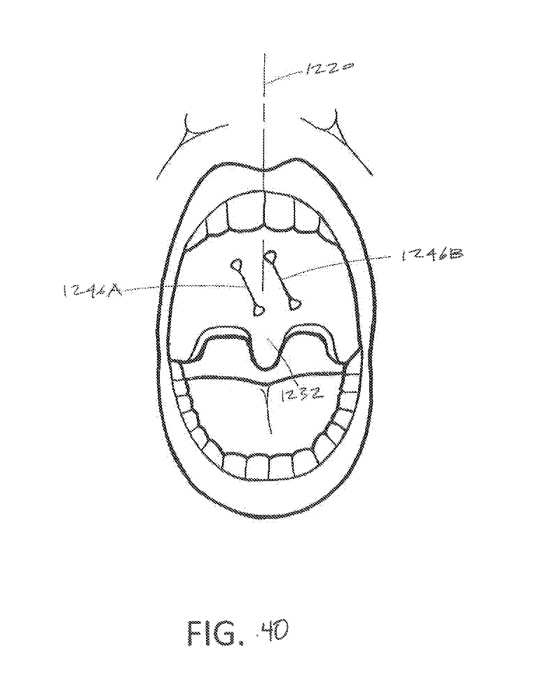

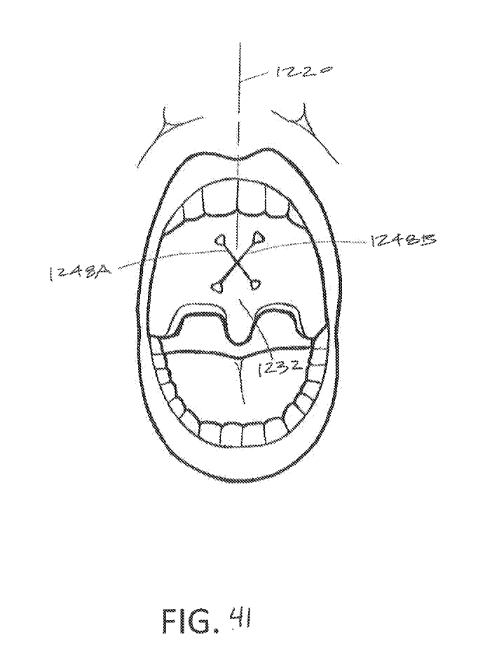

FIG. 40 illustrates another variation in which first and second implants 1246A and 1246B are implanted in the soft palate 1232 with axes that are parallel with each other but have an angled orientation relative to the mid-line 1220. The variation of FIG. 41 depicts first and second implants 1248A and 1248B implanted in the soft palate 1232 with axes that cross one another and are angled relative to the mid-line 1220. Implants that cross one another may contact each other or may cross over one another (e.g. may appear to cross each other if viewed from the top (head) of the patient).

### F. Implant Delivery Method, Systems, and Devices

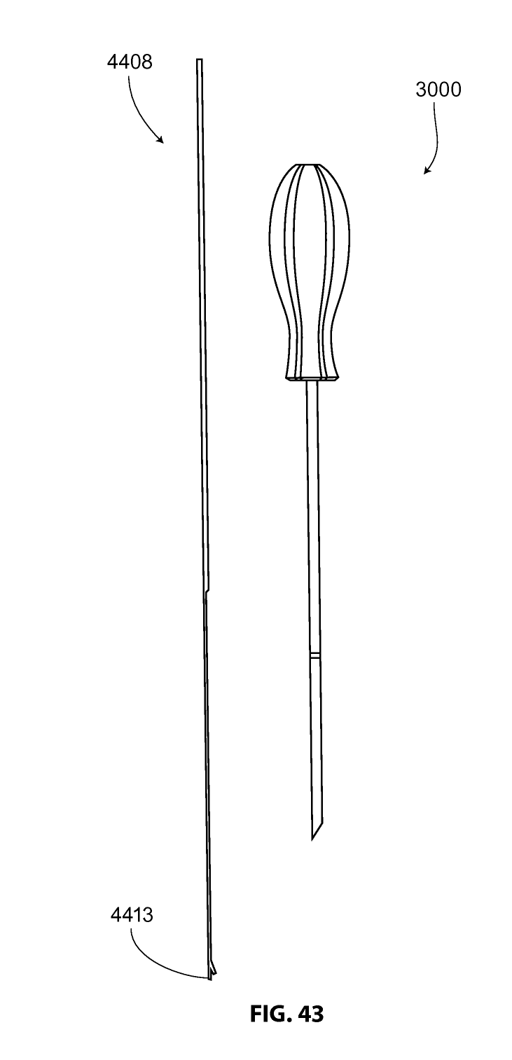

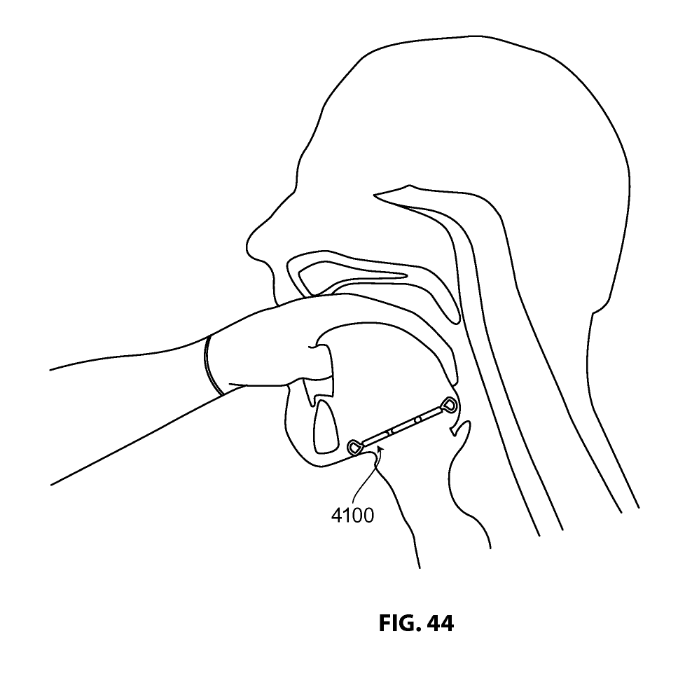

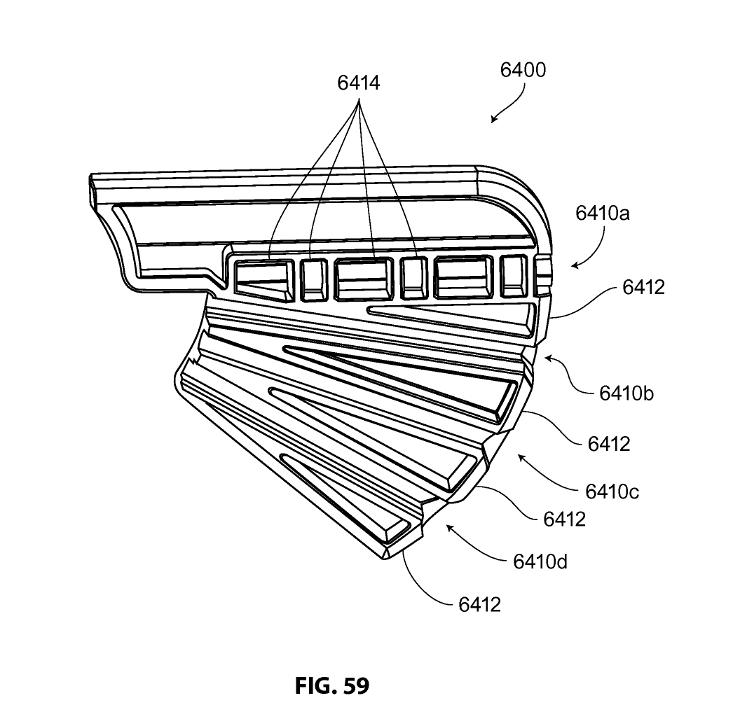

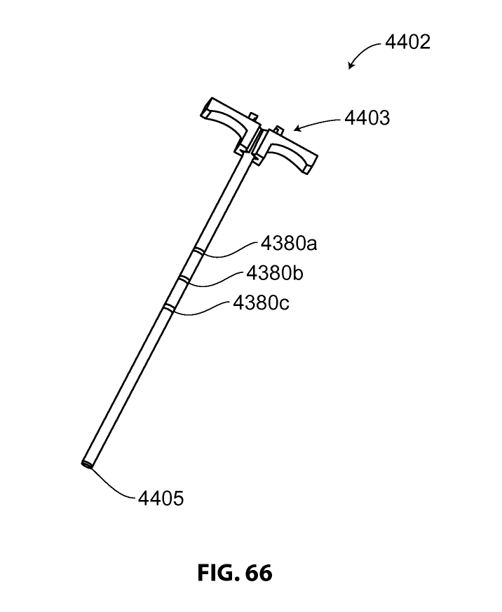

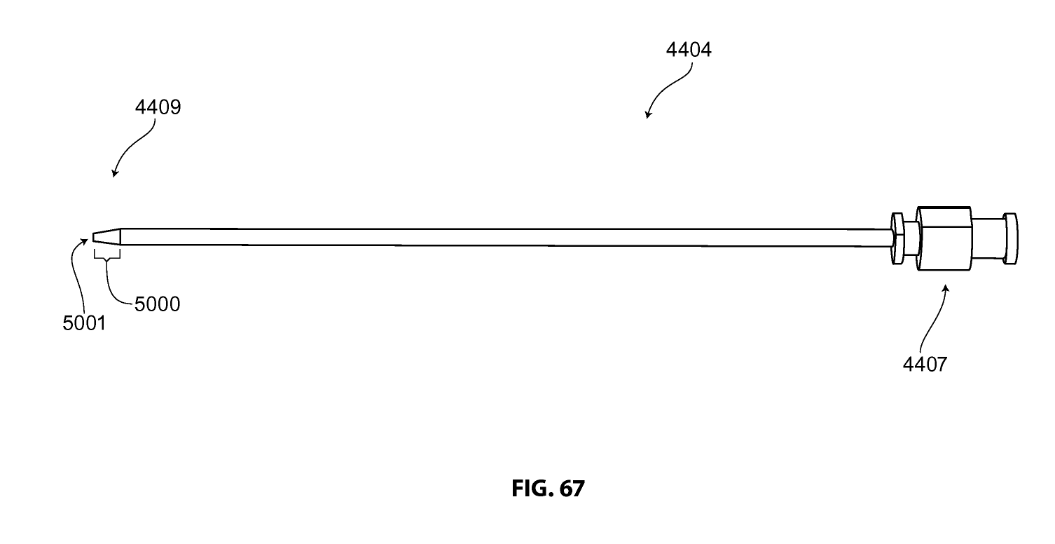

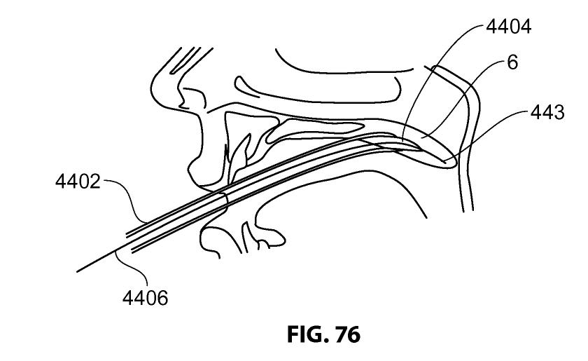

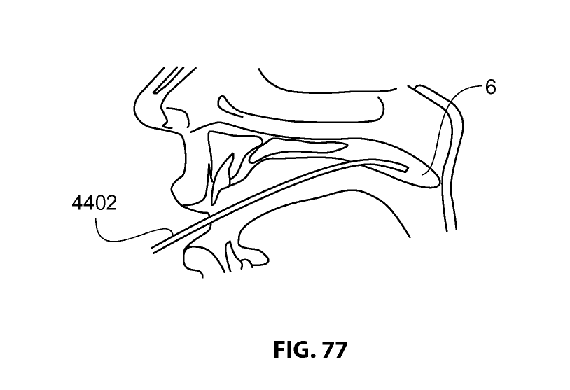

Now turning to FIGS. 30-34 and FIGS. 42-89K, various aspects of the invention are described that relate to placement of the implants within the tongue or soft palate of the patient. Implantation may be achieved in a variety of manners, and has been typically accomplished by the insertion of a needle-based cannula 760 as shown schematically in FIG. 30. It should be appreciated that an open surgery or other minimally invasive surgical technique can be used.

In one embodiment of sharp-tipped cannula 760 shown in FIG. 30, the implant body 770 is carried in bore 772 of the cannula. A thin push rod or stylet member 775 has a distal end 777 that releasably engages a distal portion 778 of the implant body. The engagement can comprise a hook or other attachment means for coupling with the distal end of the implant body. The stylet 775 can reside in the cannula bore 772 alongside the flexible implant body in such a manner that when said stylet is pushed, the distal end of the stylet functions pull or deploy the implant 770 through said cannula, avoiding any jamming or bunching of said implant during deployment. Further, the implant can be deployed in the targeted tissue site in a fully elongated (i.e. non-bunched) fashion. In another aspect of the method, the cannula is introduced into the targeted site, and thereafter the physician maintains the stylet 775 in a fixed position and contemporaneously withdraws the cannula 760 to thus deploy the implant body 770 in the targeted site.

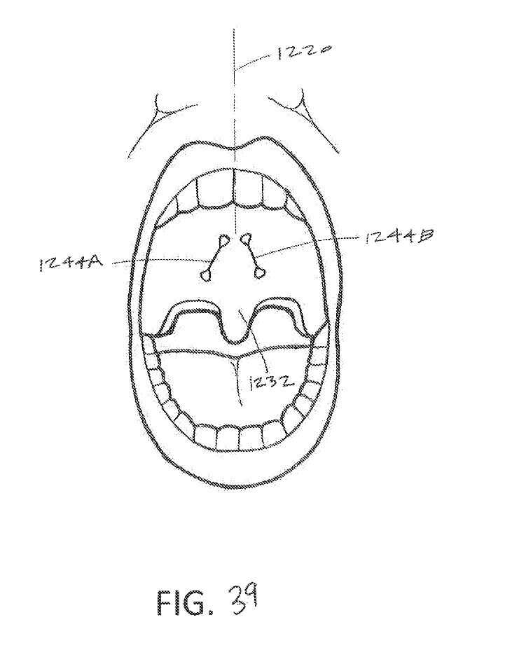

The disclosed implants may be placed within the tongue by means of straight, curved, articulating, deformable or telescoping cannulas 760 as in FIGS. 30-34, which may be introduced through any access points described above. The route of access to the implantation site within the tongue may include access via a sublingual location as depicted in FIGS. 30 and 32A-32B, (within the oral cavity, below the anterior portion of the tongue), access via a submandibular location as depicted in FIGS. 33-34 (below the anterior portion of the mandible), access via a posterior lingual location (on the posterior surface of the tongue) or any other access point that may allow for proper implant positioning.

The route of access to the implantation site within the soft palate may include access via an intra-oral location (within the oral cavity adjacent to the junction of the soft palate and the hard palate) or an intra-nasal location (within the nasal cavity adjacent to the junction of the soft palate and the hard palate), or any other access point along the soft or hard palate that may allow for proper implant positioning.

In one example, FIG. 30 shows a straight cannula inserted in the sublingual location, resulting in a substantially straight placement with the anterior anchor located adjacent to a superior part of the mandible. In another example, FIGS. 32A-32B depict an angled, bendable, or articulating cannula 780 with a telescoping secondary cannula 782 inserted in the sublingual location which would result in a substantially straight implant placed with the anterior anchor portion of the implant located adjacent to a superior part of the mandible.

FIG. 33 depicts a straight cannula 760 inserted in the submandibular location which would result in a substantially straight implant placement with the anterior anchor located adjacent to an inferior part of the mandible. In another example, FIG. 34 shows a curved cannula inserted from a submandibular location which results in a slightly curved position with the anterior anchor located adjacent to a mid-level position on the mandible.

In another embodiment, the second sleeve may have memory shape (e.g. NiTi) or may be a plastic sleeve.

Additionally, the disclosed implants as described above are substantially flexible, and are typically fabricated of flexible and/or elastic materials such as silicone, urethane, fluoroelastomer, or other bio-compatible elastomers, polyethylene terephthalate (e.g. Dacron®) or other fibers, bioabsorbable polymers, flexible metals or the like. The flexibility of the implants allows for such implants to be easily deployed and implanted through small cross-section cannulas, which may be straight, curved or articulated, without the implant body jamming within the cannula bore. Longer implants may be delivered through curved or bent cannulas than would be possible with stiff or rigid implant materials or designs.

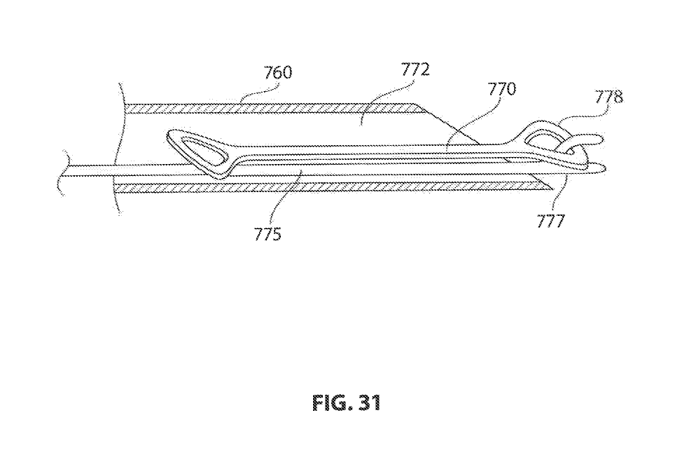

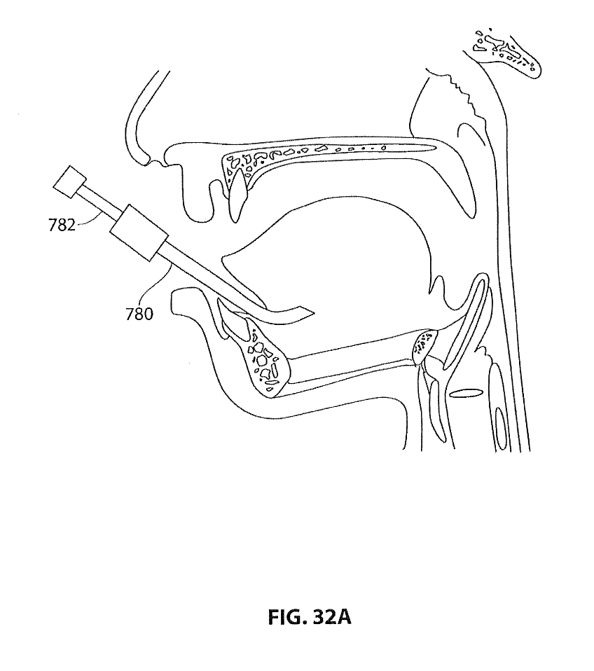

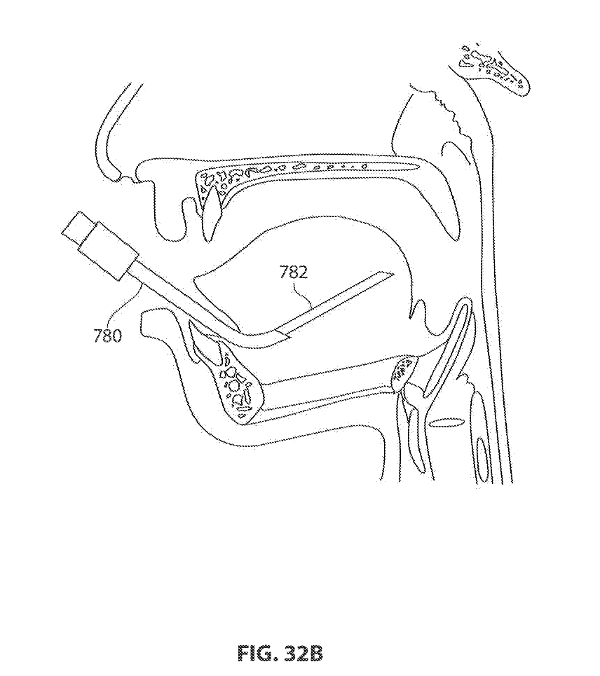

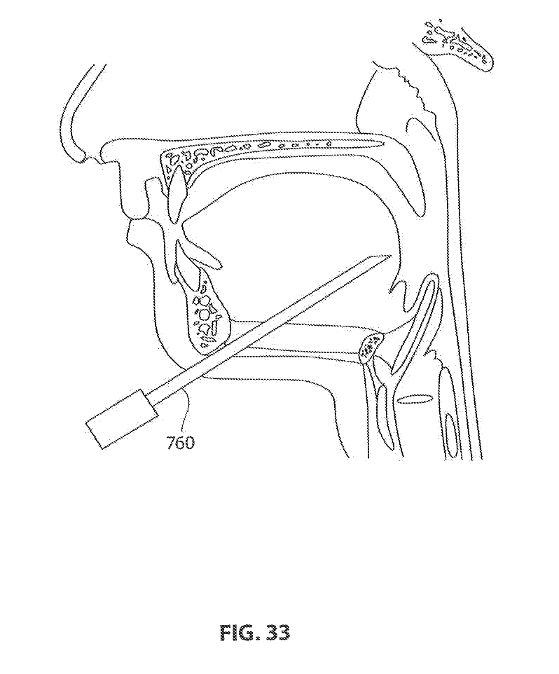

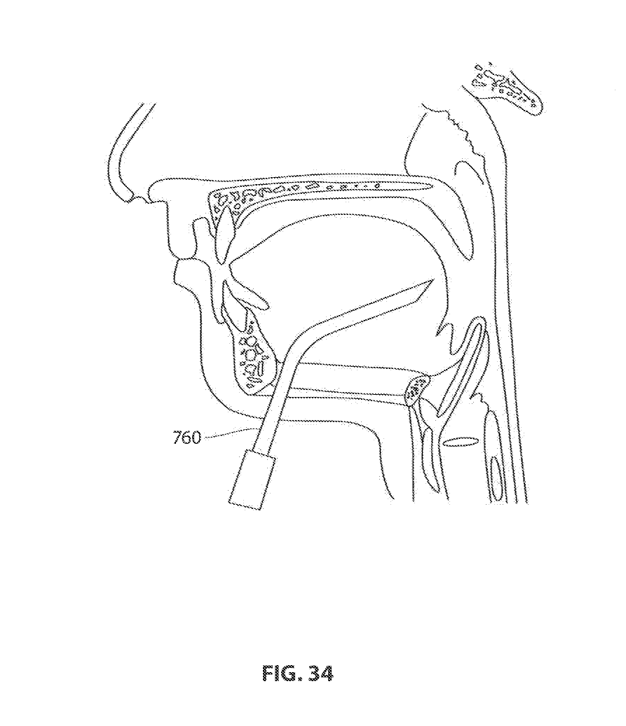

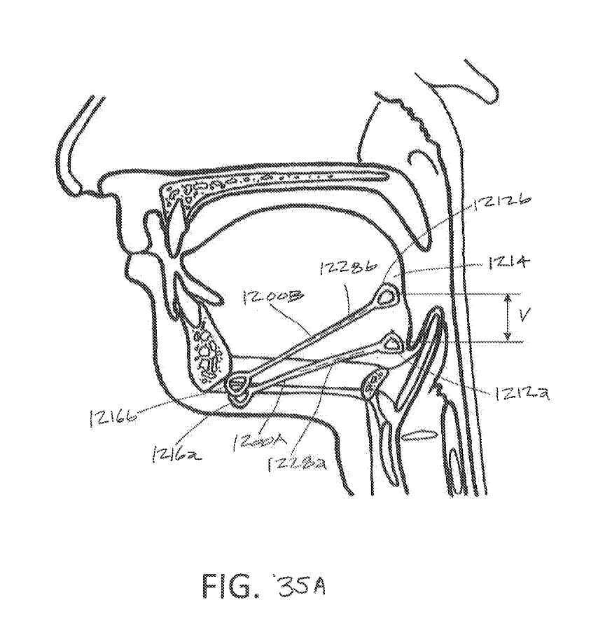

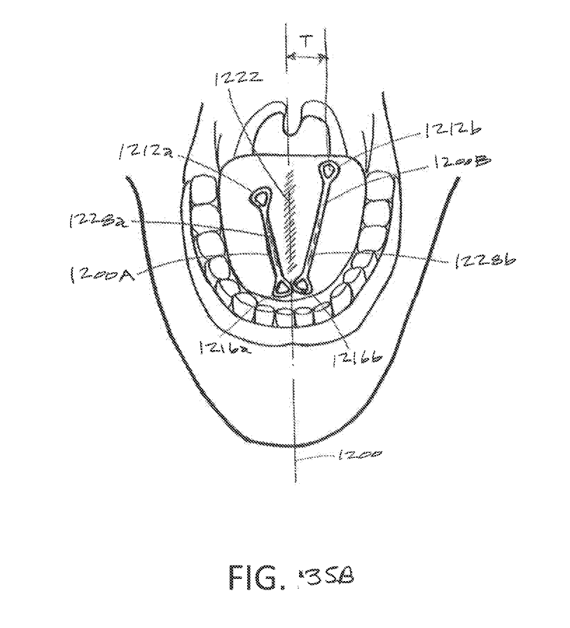

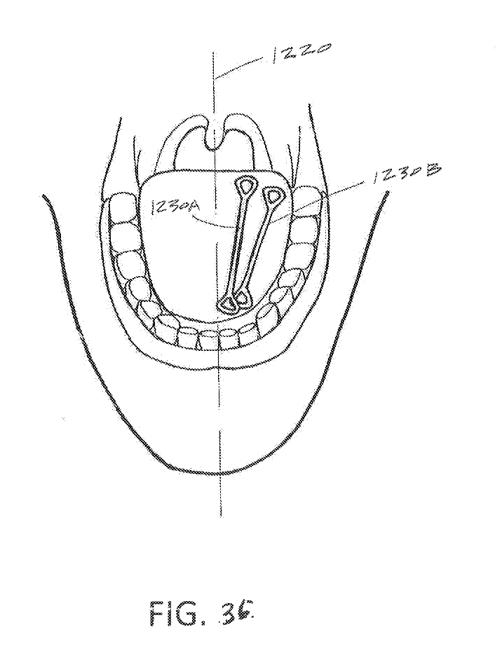

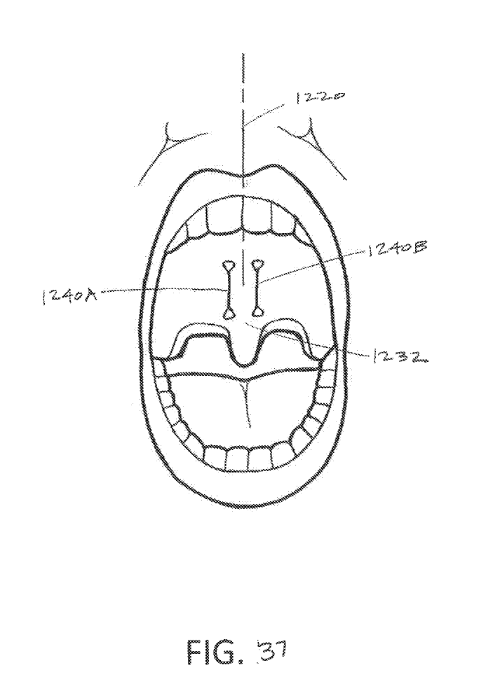

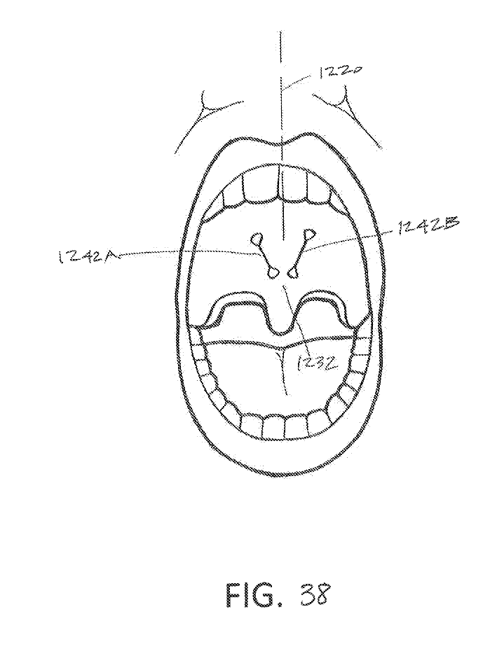

Because such implants are substantially flexible, pulling the implants, instead of pushing them, through the cannulas may be advantageous for certain applications, such as narrow, straight, curved, deformable or articulated cannulas. The primary advantage of pulling or deploying a flexible implant from such a curved or straight cannula is an increased resistance to bunching, buckling, or otherwise jamming in the cannula bore. This aspect of the deployment method allows such flexible implants to be delivered around tight bends in the cannula, thus enabling implantation in difficult to reach locations such as delivery within the tongue through the sublingual space (see FIGS. 31-32B). Pulling also allows longer implants to be delivered than would otherwise be the case. In another embodiment, only the end portions of the implant are deformable.

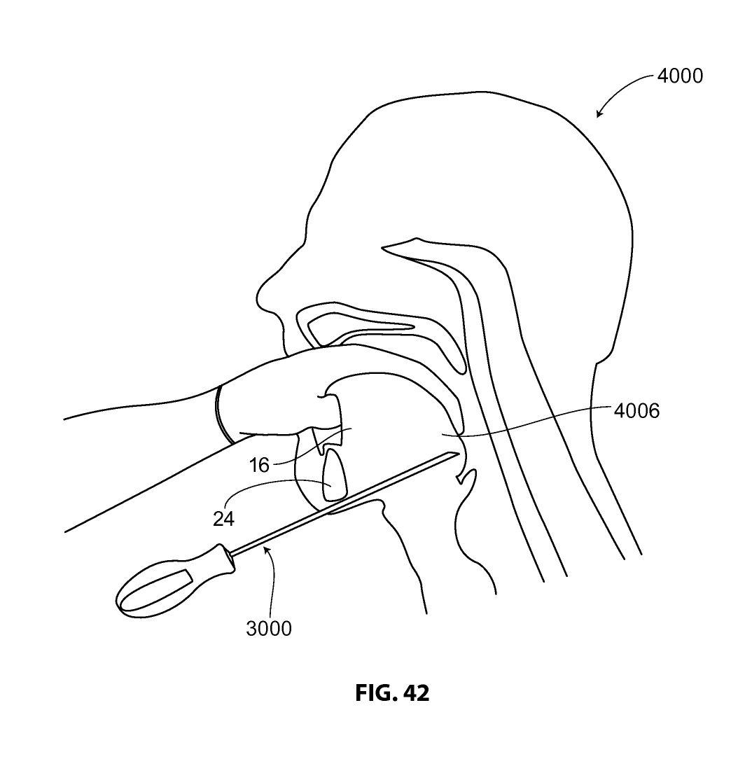

Referring to FIGS. 42-44, a trocar 3000 can be used to deliver an implant into an implant position within the patient's tongue 16. As shown, the trocar is inserted through an incision near the jawline of the patient 4000 and into the tongue tissue to position the tip of the trocar near the base 4006 of the tongue 16. In some cases, the physician (or other medical professional performing the implantation process) places his hand in the patient's mouth and on the tongue 16 to provide an opposing force against the insertion force of the trocar. This allows the physician to insert the trocar tip to a sufficient tissue depth. Additionally, the physician's hand and fingers may be able to feel the trocar tip through the base of the tongue. This tactile feedback allows the physician to determine if the trocar tip is positioned at the desired location, which may correspond to the desired location of an implant. In some cases, the insertion force for inserting the trocar into the patient's tongue is about 2 lbs to 2.5 lbs of force.

The trocar 3000 may include a lumen through which an implant body can be received and delivered to a target tissue site. As described above, the implant may be deployed by pusher 4408 (e.g. push rod or stylet) that engages a portion of the implant 4100. The pusher 4408 may include a distal end adapted to releasably engage an end of the implant 4100. Once engaged, the pusher 4408 can move the implant through the trocar lumen and out of the tip of the trocar 3000 positioned near the base of the tongue. The pusher 4408 can disengage the implant 4100 once the implant 4100 has been placed into the target position within the tongue 16. Following implant deployment, the pusher and trocar are removed from the patient. As can be appreciated, FIGS. 42-44 show implant delivery in the tongue for illustration purposes only and does not limit the use of the delivery system or methods to a particular airway tissue.

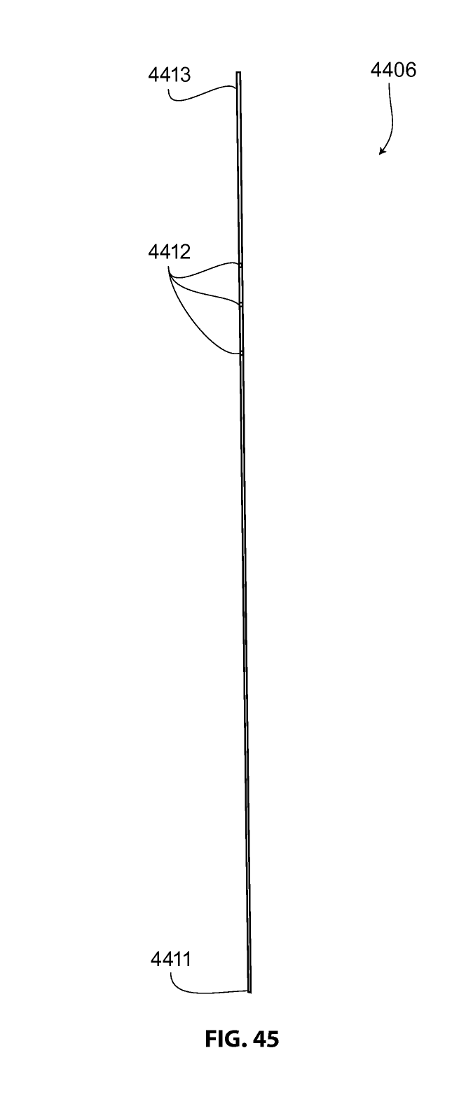

Additionally, although a trocar or cannula as described above can be used effectively to place one or more implants into target tissue sites for treatment of airway disorders, in another embodiment, an implant delivery system having one or more wires provides an alternative delivery approach. FIGS. 45-48 depict an example of a multi-wire delivery system having at least one wire 4406 adapted for insertion into airway forming tissue such as the soft palate 6 or tongue 16. As shown in FIG. 45, a delivery wire 4406 may include a wire body having a distal end 4413 and a proximal end 4411. In some embodiments, the wire body is an elongate body that may be stiff or flexible along the length of the wire body. The elongate body may be sufficiently stiff to maintain a straight or linear orientation when partially positioned within an airway tissue. In some cases, the wire is sufficiently stiff to maintain a straight or linear orientation while dissecting through tissue. In other cases, the wire may be sufficiently flexible to allow at least a portion of the wire body to curve or bend to accommodate rounded or curved tissue structure. For example, the wire body may include a flexible portion configured to align with a curvature of an airway tissue as the wire is advanced into the airway tissue.

Advantageously, the contemplated wire may be adapted to provide minimally invasive insertion through and into the airway forming tissue. In some cases, this may be accomplished by including a suitable distal end 4413 having a tip sufficient for cutting through the airway forming tissue without perforating, coring, or otherwise injuring mucosal and dermal tissue. For example, the distal end 4413 may be tapered, rounded, beveled, domed, or otherwise shaped to allow dissection through tongue tissue without allowing the tip to cut through tougher or thicker mucosal or dermal tissue at the base of the tongue, which could lead to the tip perforating and injuring the patient's pharynx, causing a possible infection risk, or making the precise placement of the implant near the tongue surface more difficult.

Depending on the target treatment site and airway forming tissue, the wire tip shape may be blunt, domed, sharp, beveled, lancet-shaped, tapered, rounded, etc. In some cases, the wire has a beveled tip with a bevel angle between about 30 to about 60 degrees. In some cases, the beveled tip has a bevel angle about 45 degrees. In other embodiments, the wire has a tapered tip with a taper angle of about 15-30 degrees per side. In some cases, the tapered tip has a taper angle about 20 degrees.

In additional variations, the wire may have a diameter or cross-sectional dimension between about 0.10 inches to about 0.60 inches. For example, the wire may have a diameter of about 0.18 inches, about 0.020 inches, about 0.024 inches, about 0.025 inches, about 0.037 inches, about 0.040 inches, about 0.050 inches, and about 0.060 inches.

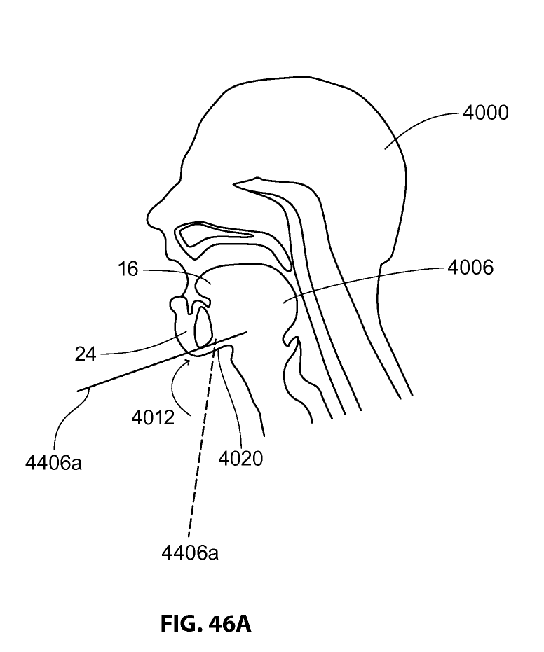

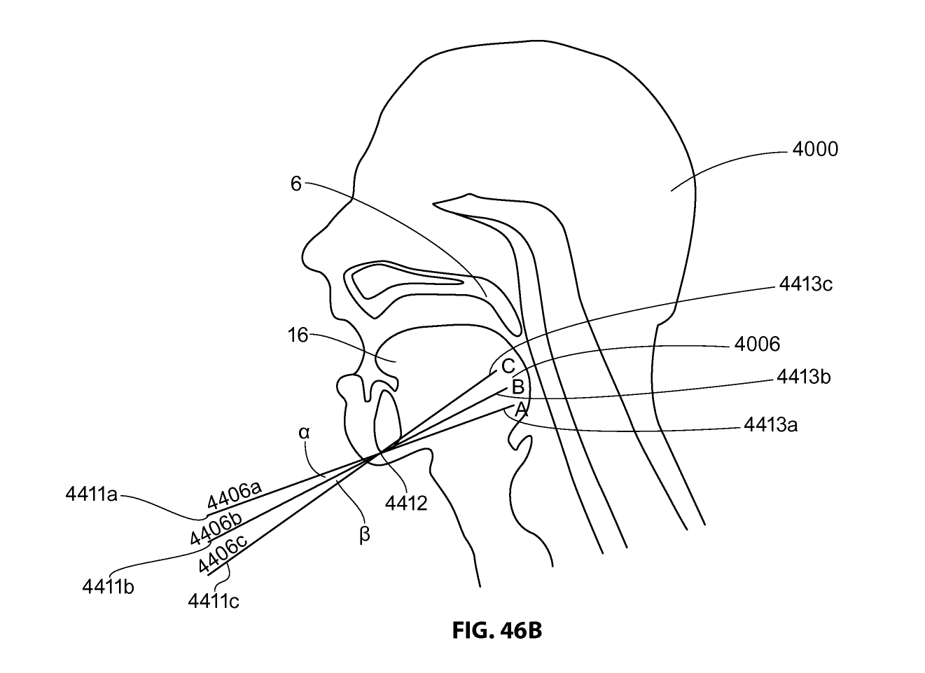

The wire may also have any suitable length depending on the expected depth of expected tissue penetration and ease of manipulation. As shown in FIG. 46A, the wire 4406a may have a sufficient length allowing a portion of the wire 4406a to be inserted into the patient's tongue while another proximal portion of the wire resides outside of the patient's body. The wire 4406a is partially inserted through an incision 4020 near the mandible 24 to a first wire position shown in dashed line. The physician then adjusts the wire 4406a to a second wire position (shown in solid lines) by manipulating the proximal portion of the wire remaining outside of the patient's body. In the example, the physician pivots the wire 4406a against the patient's chin to adjust the position of the wire 4406a.

Advantageously, the wire length may also be sufficient to include at least one marker 4412 (FIG. 45) for indicating tissue penetration of the wire. In some cases, the wire length is between about 8 cm and 40 cm. In other cases, the wire length may be between about 8 cm and about 25 cm.

The physician may also apply force to further insert, drive, or push the wire into the target tissue. This may be accomplished by applying an insertion force on the proximal portion of the wire residing outside of the patient's body to distally drive the wire further into the target tissue. In some cases, the amount of force used to insert the wire into tissue is between about 1 lb to about 3 lbs of force. In other cases, the force is about 1.15 lbs of force. In other cases, the force is less than about 1.5 lbs of force.

Once the wire is in a desired position and orientation, the wire defines a delivery pathway for an implant into the target tissue site. The wire may also define an implant position in the tissue site based on the wire's own orientation in the tissue. FIG. 46B depicts an example of a plurality of wires used to define a plurality of implant positions within the patient's tongue. As shown, a first wire 4406a, a second wire 4406b, and a third wire 4406c are partially inserted into the tongue 16. Each wire includes a distal end 4413a-c respectively that corresponds with a target site for implant delivery. Additionally, each wire defines an implant delivery path along the length of the wire from outside the patient into the tongue.

In operation, the placement of each wire relative to each other wire in the tissue may be determined and set prior to implant deployment. For example, a physician may insert the first wire 4406a having a first wire location in the tissue that defines a first implant delivery path to deliver a first implant to a first implant position A in the patient's tongue. The physician can then insert a second wire 4406b having a second wire location in the tissue that defines a second implant delivery path to deliver a second implant to a second implant position B. Continuing with this example, a third wire 4406c having a third wire location is inserted into the tissue and defines a third implant delivery path to deliver a third implant to a third implant position C. Any and all of the wires 4406a-c may be placed in respective positions and location in the tissue prior to deployment of any of the implants to the implant positions at the target tissue site(s). Additionally, the physician may adjust the position of any of the wires 4406a-c relative to each other to adjust the corresponding implant position defined by a wire.

In some embodiments, the wires provide tactile feedback indicating depth of tissue penetration. The physician may place his hand along the tongue with fingers on the base of the tongue. The wire may be configured to allow the physician to feel the tip of the wire through a tissue thickness at the base of the tongue to determine if proper tissue depth has been achieved.

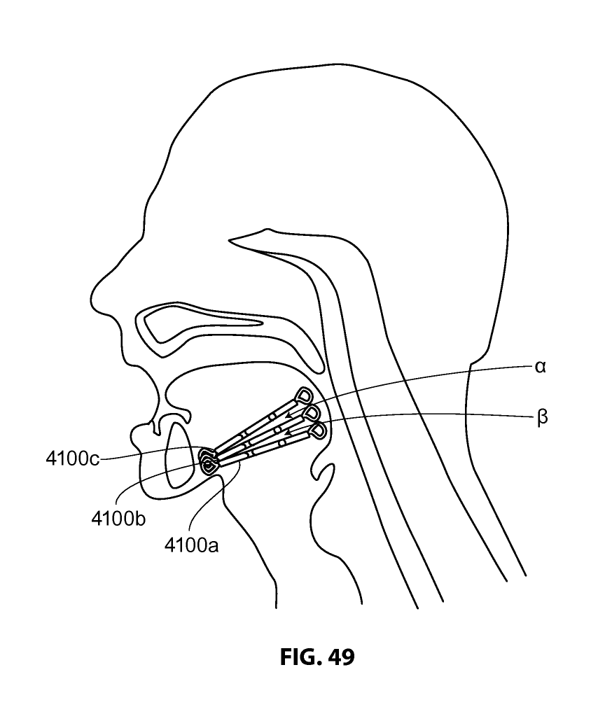

Referring to FIG. 49, three implants 4100a-c are delivered along delivery paths defined by respective wires 4406a-c. The angles between the wires 4406a-c correspond to the angles between the implants. Similarly, the spacing between the wires also reflects the spacing of implants relative to one another.

In some variations, the angle between wires may be between about 0 degrees to about 60 degrees. In other embodiments, the angle is between about 0 degrees to about 45 degrees. In further embodiments, the angle is between about 10 degrees to about 30 degrees. In additional embodiments, the angle is between about 15 degrees to about 45 degrees. As used herein, angle may refer to the angle between two wires or to the total angle between more than two wires (e.g. total angle). Additionally, the wire may be spaced apart by a distance which can be at least about 0.25 cm, at least about 0.50 cm, at least about 1 cm or at least about 1.5 cm.

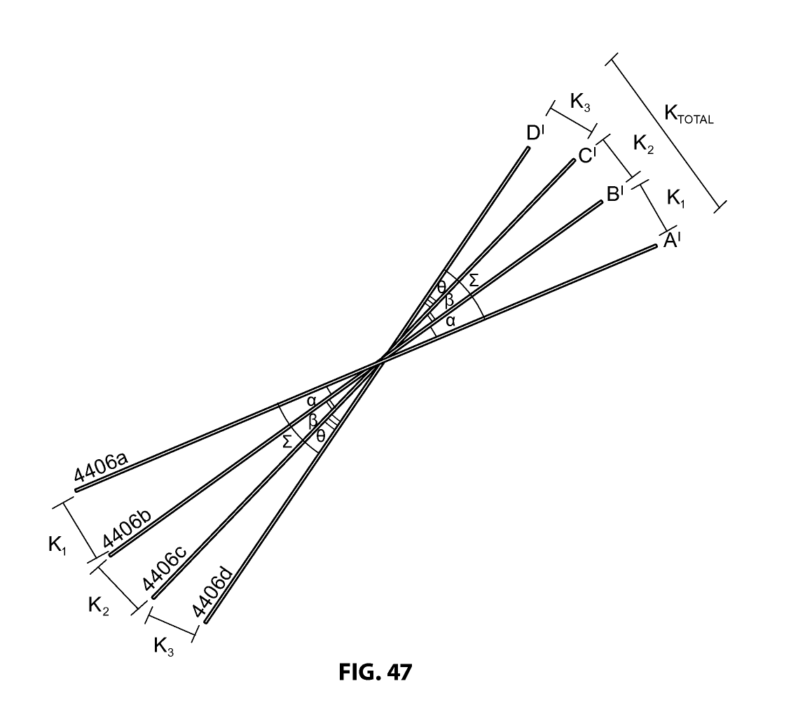

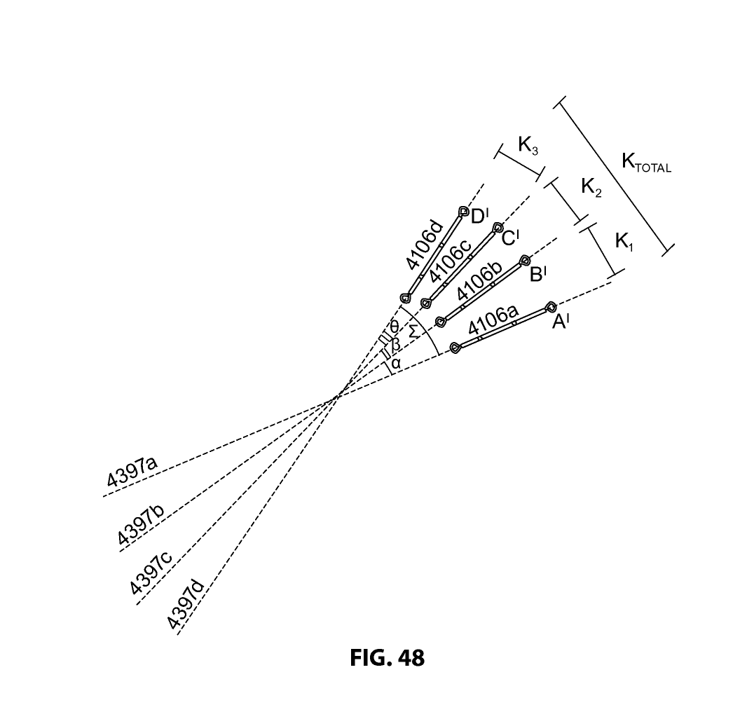

FIGS. 47-48 provide an example with four wires and four corresponding implant positions (A′, B′, C′, D′) defined by the placement of the four wires. As shown in FIG. 47, a first wire 4406a and a second wire 4406b are separated by an angle alpha α and a distance K1. The second wire 4406b and third wire 4406c are separated by an angle beta β and a distance of K2. Likewise, the third wire 4406c and fourth wire 4406d are separated by an angle theta θ and a distance of K3. In some variations, the wires may define a total angle Σ that is a sum of all angles between the wires. Similarly, a total separation distance between the wires may be represented by a total Ktotal.

Referring to FIG. 48, the position of the wires in FIG. 47 corresponds to the implant positions of implants delivered along a delivery path of a specific wire. For example, a first implant 4100a delivered along a delivery path of first wire 4406a will be placed in implant position A′. A second implant 4100b delivered along a delivery path of second wire 4406b will be placed in implant position B′. Moreover, implant position A′ and implant position B′ will be set apart by an angle alpha and a distance K1.

The positional relationship between the third implant and fourth implant will also correspond with the wire orientations. That is, the second implant position B′ will be set apart by an angle beta and distance K2 from the third implant position C′. The third implant position C′ will be set apart by an angle theta and distance K3 from the fourth implant position D′. Likewise, a total angle will be formed by the sum of the angles between the implant positions A′-D′ and a total dimension Ktotal will separate the implant positions collectively.

By setting the wire placement prior to implant deployment, the physician can precisely determine the approximate implant positions for multiple implants prior to delivery. Where a proximal portion of the wires reside outside the patient, the physician can visually confirm the orientation of wire portions inside the patient as the proximal portions outside the patient correspond to the portions inside. The implants can then be introduced into the implant positions with the proper spacing, separation, and orientation relative to one another to provide optimal therapeutic treatment.

In further embodiments, the wire may include a wire axis extending along a length of the wire. In some cases, the wire axis defines an implant position within tissue. Where multiple wires are employed, each wire may define a wire axis. As shown in FIG. 48, a first wire may define a first wire axis 4397a and a second wire may define a second wire axis 4397b. The first and second wire axes may be aligned, parallel, non-parallel, vertically or horizontally staggered, and otherwise orientated as suitable for defining an implant position with the patient's target tissue site. FIG. 48 shows an additional third and fourth wire axis 4397c-d for the third and fourth wires.

As described, the angle between the wires or wire axes may be between about 0 degrees to about 60 degrees. In other embodiments, the angle is between about 0 degrees to about 45 degrees. In further embodiments, the angle is between about 10 degrees to about 30 degrees. In additional embodiments, the angle is between about 15 degrees to about 45 degrees. As used herein, angle may refer to the angle between two wires or to the total angle between more than two wires (e.g. total angle Σ).

Additionally, the wire, wire axis, implants, and/or implant positions may be spaced apart by a selected dimension K, which may be a distance between positions. In some variations, K is at least about 0.25 cm, at least about 0.50 cm, at least about 1 cm or at least about 1.5 cm. In some variations, the total distance between wires, axes, implants, or positions is at least about 0.25 cm, at least about 0.50 cm, at least about 1 cm or at least about 1.5 cm.

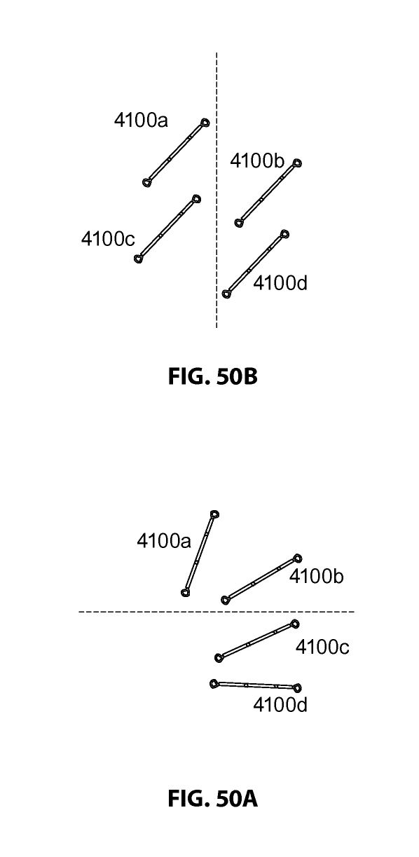

As can be appreciated, any number or variations of suitable implant positions may be appropriate for treatment of airway disorders. As such, the placement of multiple wires may be adjusted to correspond to desired implant positions. As shown in FIG. 50A, the implants may have a horizontally offset or staggered orientation (e.g. not parallel) relative to one another where each implant is angled relative to a horizontal plane (dashed line). Furthermore, FIG. 50B shows vertically offset or staggered implants relative to a midline plane. Two implants 4100a,c are to the left of the midline plane (dashed line) while two implants 4100b,d are to the right of the midline plane (viewing from the posterior or base of tongue).

In any of the contemplated embodiments, any type of wire may be suitable for delivering the implant to the target site. In some cases, the wire may be a solid stylet, needle, or a combination of a stylet and a needle. Additionally, the wire may be coated or uncoated.

To facilitate wire placement, a wire guide may be used to define and maintain wire positions. A wire guide may include a plurality of through-holes, openings, lumens, or channels with predetermined orientation and position. In some variations, the wire guide lumens may include a locking mechanism for holding a received wire in a fixed position. The wire guide may be adapted to receive and engage a wire through an opening or channel and to hold the received wire in the predetermined orientation and position. The predetermined orientation or position may include a fixed angle and/or spacing for one or more wires.

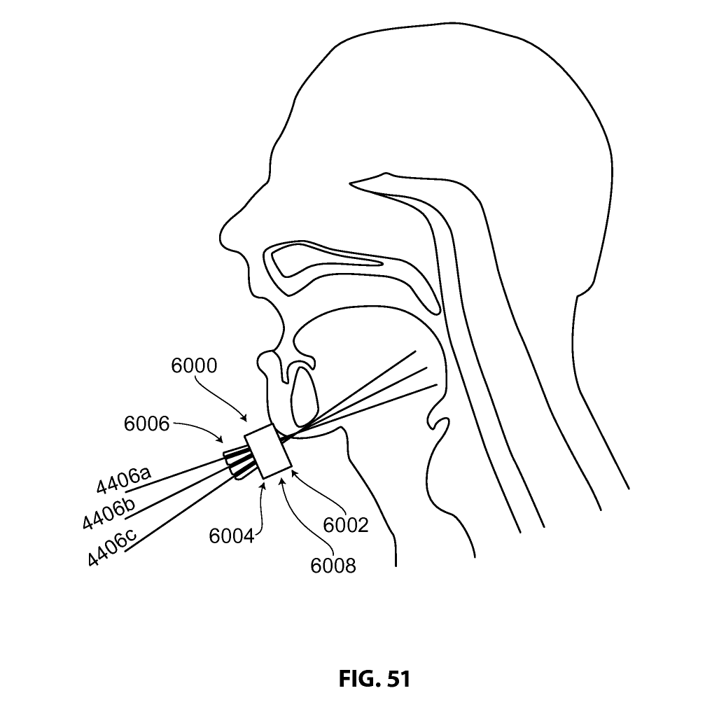

FIG. 51 shows a wire guide 6000 having a main body 6008 with a proximal end 6004 and a distal end 6002. The distal end 6002 may be adapted to interface with a patient such as by being shaped to lie against the patient's chin. The wire guide 6000 may include several channels through which wires 4406a-c can be received in order to determine the positioning of the wires relative to one another. In some embodiments, the proximal end 6004 of the wire guide 6000 may include extension members 6006 (e.g. tubing) to aid in holding a proximal portion of a wire that resides outside of the patient's body. The extension members 6006 may be made from any suitable materials including, but not limited to, Polyethylene, Silicone Rubber, Polyurethane, polypropylene, Pebax® Poly ether block amide, Teflon®, (ie PTFE Poly tetra flouro ethane and various PTFE Blends, Ethylene tri flouro ethane, Polyester and polyester blends.

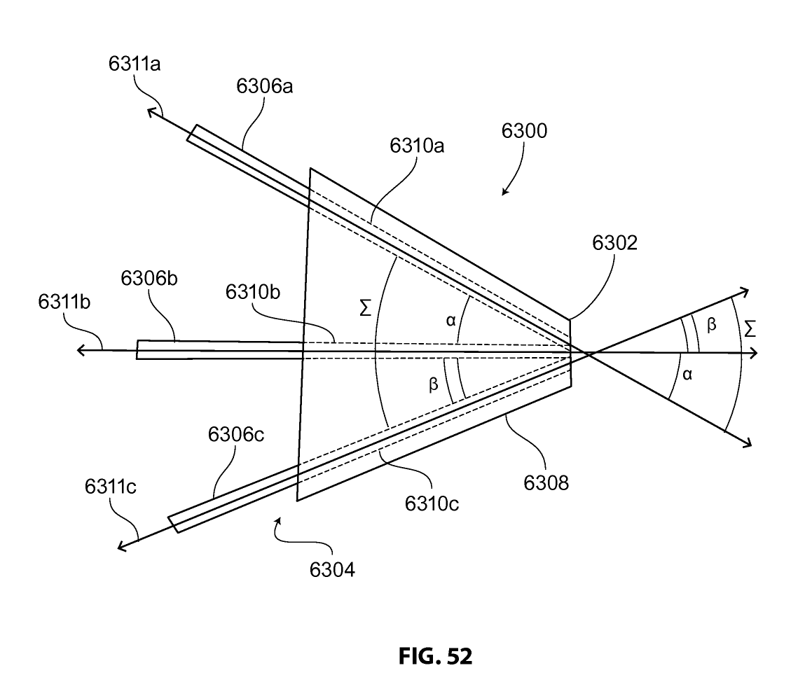

FIG. 52 shows a wire guide having a main body 6308 with a proximal end 6304 and a distal end 6302. As shown, the distal end 6302 has a smaller cross-section compared to the proximal end 6304. The main body 6308 includes three lumens or channels 6310a-c extending between the proximal and distal ends. Each lumen includes an opening at both the proximal and distal ends. Additionally, three extension members 6311a-c are coupled to the main body 6308 such that the extension members extend the length of lumens 6310a-c through the respective extension members.

Referring still to FIG. 52, each of the channels 6310a-c includes a channel axis 6311a-c extending lengthwise through the channel. The channel axes may form angles that correspond with angles formed by the channels relative to one another. For example, a first channel 6310a forms an angle alpha α with a second channel 6310b. The second channel 6310b forms an angle beta β with a third channel 6310c. Additionally, a total channel angle sigma Σ is formed by the sum of the channel angles alpha and beta. As shown, the angles for the channel axes 6311a-c correspond with the angles alpha, beta, and sigma for the channels 6310a-c. Moreover, once wires are placed into the wire guide channels, the channel axes angles (and channel angles) define the wire angles for wires in the wire guide. As such, the wire guide also guides the eventual placement and orientation of implants in the target tissue site.

In some variations, the angles may be between about 0 degrees to about 60 degrees. In other embodiments, the angle is between about 0 degrees to about 45 degrees. In further embodiments, the angle is between about 10 degrees to about 30 degrees. In additional embodiments, the angle is between about 15 degrees to about 45 degrees. As used herein, angle may refer to the angle between two channels (or axes) or to the total angle between more than two channels or axes (e.g. total angle Σ).

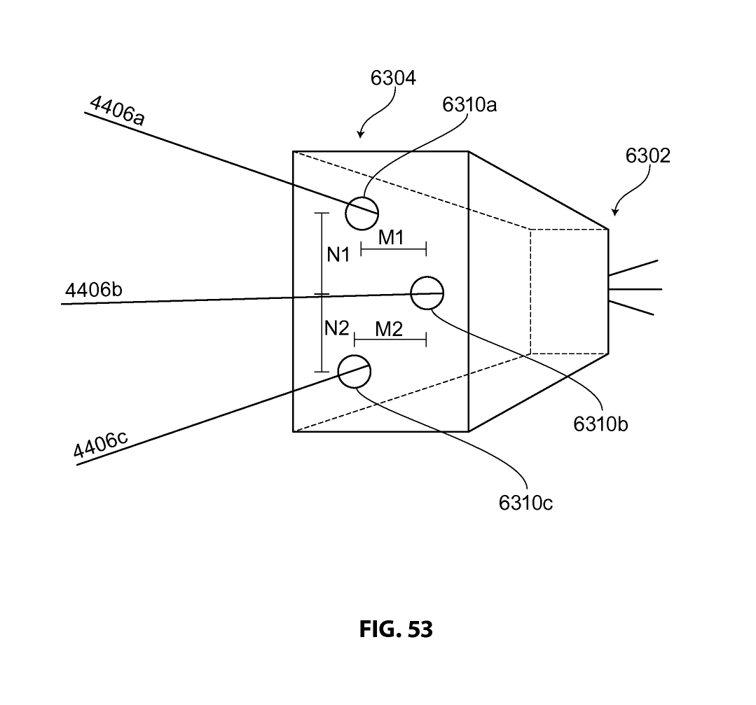

Referring to FIG. 53, the wire guide channels or lumens 6310a-c may be horizontally or vertically offset from one another. As shown, a first channel 6310a is vertically offset or staggered from a second channel 6310b by a separation dimension/distance of M1. Second channel 6310b and third channel 6310c are vertically staggered by a dimension/distance of M2. Additionally, the second channel 6310b is horizontally staggered from the first and third channels 6310b-c by a dimension/distance N1 and N2 respectively. This arrangement determines the corresponding placement of the wires 4406a-c in the wire guide channels, which, in turn, determines the corresponding implant positions in the target tissue suite that are delivered along a delivery path defined by each wire 4406a-c.

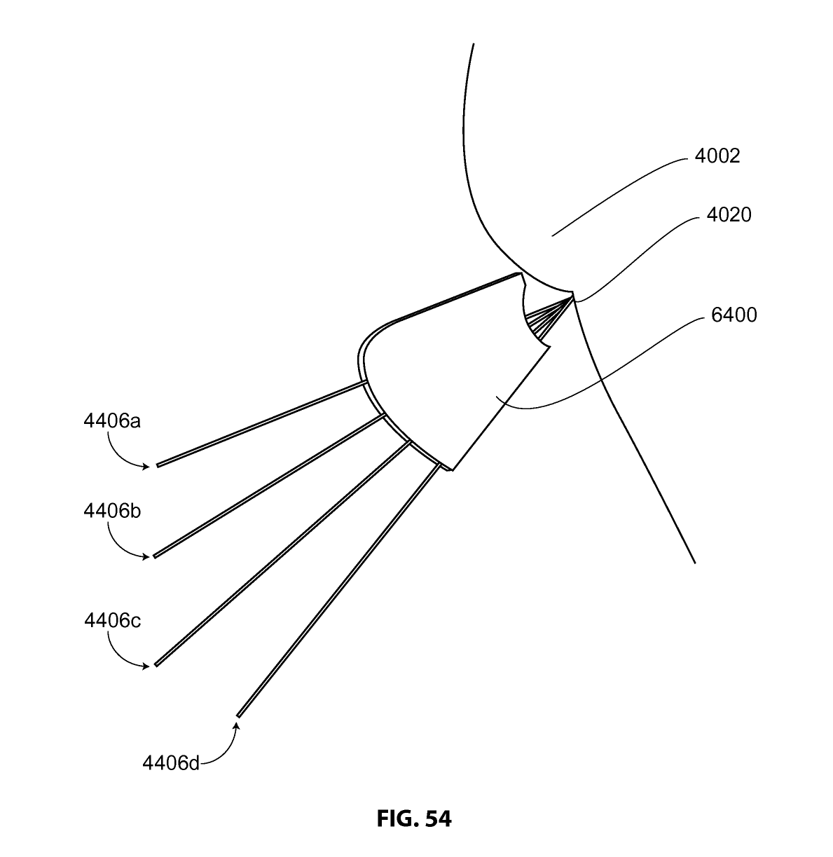

FIGS. 54-59 illustrate another example of a wire guide having lumens, openings, or channels for receiving and positioning multiple implant wires. FIG. 54 shows a wire guide 6400 with four wires 4406a-d extending through the wire guide body. The wire guide 6400 includes a distal end adapted for interfacing with the patient's chin 4002 near an incision point 4020. As shown, the wire guide 6400 is configured to receive and engage a wire within each lumen. Once received within a channel, the orientation of the wire corresponds to the orientation of the wire channel and/or a channel axis of the wire channel.

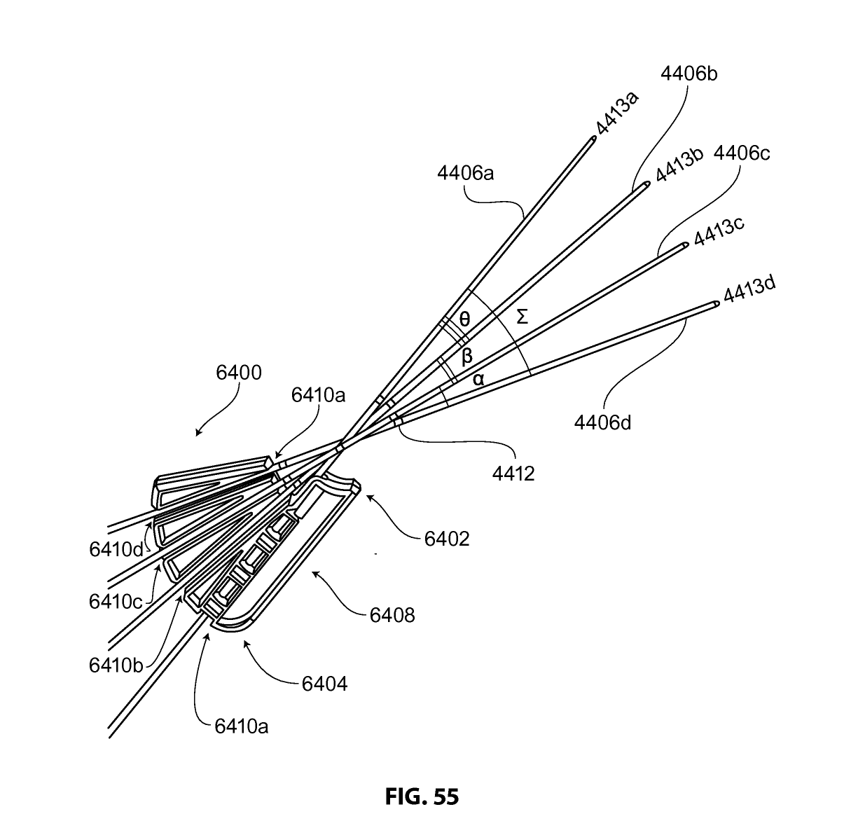

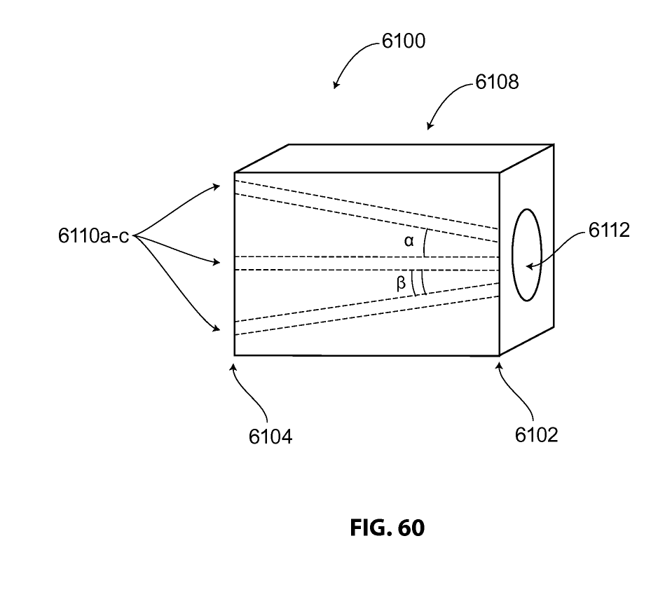

FIG. 55 shows a side view of the wire guide 6400 in FIG. 54. The wire guide 6400 has a main body 6408 with an open side. The main body 6408 includes four separate wire channels 6410a-d. Each wire channel 6410a-d includes an opening at the distal end 6402 and proximal end 6404 of the main body 6408. In some embodiments, each channel has independent openings at each end of the main body. In other cases, the channels may share an opening at one or both ends (FIG. 60).

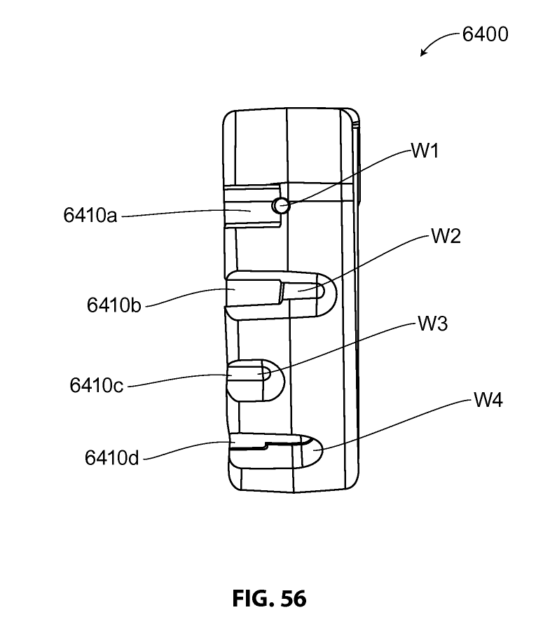

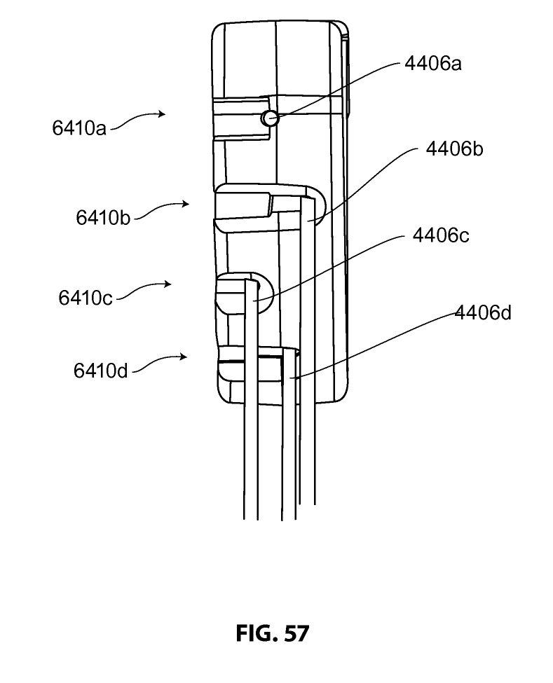

As shown in FIG. 55, it is not necessary for the channels to be uniform in dimensions. For example, first wire channel 6410a is longer than the second, third, and fourth channels 6410b-d. In some cases, the length of a channel is between about 4 cm and 5 cm or between 1 and 3 cm. The channel length may be more than two times the diameter of the wire being guided and preferably longer than 5 times the wire being guided to capture the diameter of the wire and determine its range of position relative to the desired placement angle. Channels may also have different widths W or depths relative to others. Referring to FIG. 56-57, the first channel has a width W1 that is less than the widths W2-4 of the remaining channels. In some cases, varying the width or depth of a channel allows variation for the vertical arrangement of wires (and corresponding implant positions). FIG. 57 shows the placement of wires 4406a-d in the channels 6410a-d with varying widths. The channel width variation results in a vertically offset arrangement for the wires, which corresponds to a vertically offset arrangement for the implants in situ. The channel width may be sufficient to allow a wire to pass through it but not so wide as to not precisely define the angled path of the wire or at least 2× the diameter of the wire. In cases where the length of the channel length is longer than 5 or 10 times the wire diameter a larger channel diameter may be used and still provide sufficient definition of the wire angled path. In some variations, the width of the channel is between about 1.0 and 1.5× or 1.01 and 1.2× or 1.02 and 1.05× the diameter of the wire being guidedIt is to be understood that by varying the dimensions of the wire guide, any number of suitable implant positions can be fixed to greatly improve ease of delivery and precision of implant deployment.

The channels may be partially or completely enclosed by the wire guide body. As shown in FIGS. 55 and 58-59, wire body 6408 has an open side and an enclosed side. Wire channels 6410a-d are partially enclosed in the wire body 6408 where one side of the body 6408 is open. In some variations, the open side of the wire body 6408 allows lateral release of one or more wires from the wire guide 6400. As can be seen in FIG. 57, the second, third, and fourth wires 4406b-d are received and engaged in a section of the channel inward from the open side of wire guide. To release the second, third, and fourth wires from the wire guide, the wire guide may be tilted, turned, or rotated to slide the wires 4406b-d out of the open side of the wire guide body 6408. In some cases, the wires 4406b-d may be moved out of the wire guide without any rotational movement. As can be appreciated, the wires may also be placed into the wire guide from the openings on the proximal and distal ends or by sliding the wires into channels having an open side.

As shown in FIGS. 55-59, the first channel is designed to laterally retain a received wire. A plurality of struts 6414 extend along a width of the first channel to laterally hold a received wire. In operation, the first channel may be designed to laterally retain a wire in order to keep the wire guide on at least one of the wires during use. Although shown as having one laterally retaining channel and several laterally releasing channels, any combination or variation of channels may be used. Moreover, the laterally retaining mechanism may be any suitable mechanism including through holes in a 2-piece wire guide which snaps together to determine the channels and comes apart to release the wires, a single piece clam shell configuration which snaps together to form the wire channels, slots, dimples on opposing sides of the wire diameter which when the wire is inserted between them, determine its path. The wire channels may contain a tapered insertion point to facilitate easy insertion of the wire into the channel or slot.

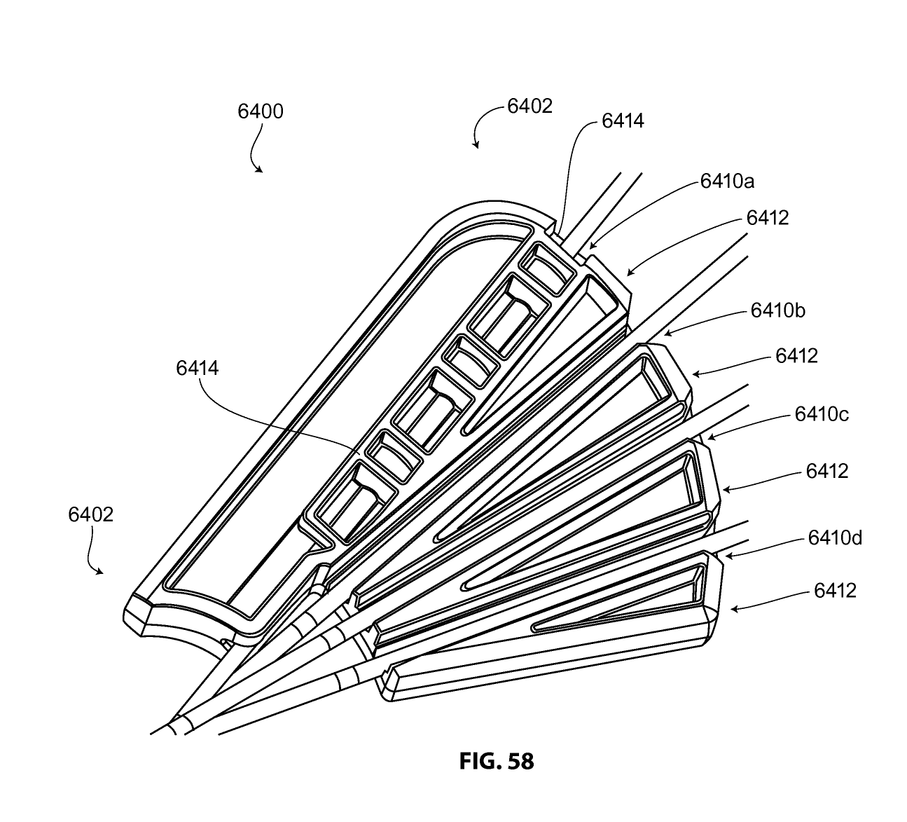

As described above, the wire guide channels may include varied dimensions to facilitate wire and implant placement in a patient's airway forming tissue. In some cases, the wire guide includes spacing structure that maintains the angle, spacing, etc. between channels and wires. FIG. 58 shows spacers 6412 between each channel. In some cases, the spacers include a sloped surface defining a portion of the channel. The sloped surface may be sloped at an angle that defines the channel angle. Additionally, a cross-sectional dimension of the spacers may define the separation between wires, and thereby the separation between implant positions at the target tissue site.

As can be appreciated, the shape of the wire guide can be any suitable shape including a triangular, circular, oval, rectangular shape, hexagonal or octagonal. FIG. 60 shows a wire guide 6100 with a rectangular wire guide body 6108 having a proximal end 6104 and a distal end 6102. Channels 6110a-c extend from openings in the proximal end 6104 to a single opening 6112 at the distal end 6102.

In operation, a wire guide may be used to facilitate placement of wires and implants in a target treatment site. In some cases, a first wire is partially inserted into a patient's airway tissue such as the tongue. Once the first wire is partially inserted, a wire guide may be placed over the first wire. The first wire channel may include a retaining mechanism to hold the wire guide onto the first wire. A second wire may then be partially inserted into the tissue and moved relative to the first wire to form an angle or distance of separation between the wires. The angle or distance of separation between the wires may be defined by a first and second wire channel in the wire guide. For example, the second wire may be received in a second channel of the wire guide where the first and second channels have a preset and pre-arranged channel angle set by spacers or other structure on the wire guide. Because the wire guide fixes the relative position of engaged wires, the guide may be used to arrange wires with any suitable angles, alignment, orientation, spacing, etc.

In some embodiments, once the wires are inserted and arranged according to the wire guide, the wire guide may be removed from the wires. The wires may be disengaged from the wire guide by rotating the wire guide to allow retained wires to laterally release from an open side of the wire guide. In some variations, where one or more the wires may be laterally retained by a channel, the wire guide may be removed from the laterally retained wires by passing the wire guide along a length of the wire and off the proximal end of the wire.

Additionally, as can be appreciated, the number of wires, implants, and channels may be varied according to treatment needs. In some cases, one to four implants may be delivered to a patient, which may require a corresponding number of wires and wire guide channels. In some cases, a wire guide may have more channels than the number of wires used for a particular procedure.

In another aspect, the placement of implants and/or wires may be facilitated by a tissue template or stencil. The tissue template may include one or more position indicators to assist in the delivery of implants into desired locations within airway forming tissue. For example, the tissue template may include one or more openings through a thickness of the template that allows a physician to use tactile feedback during an implant delivery process. In practice, the physician may place his fingers or hands on the openings during delivery to tactilely confirm the presence of a tip for a stylet, wire, introducer, or other delivery tool near the opening of the template through tissue.

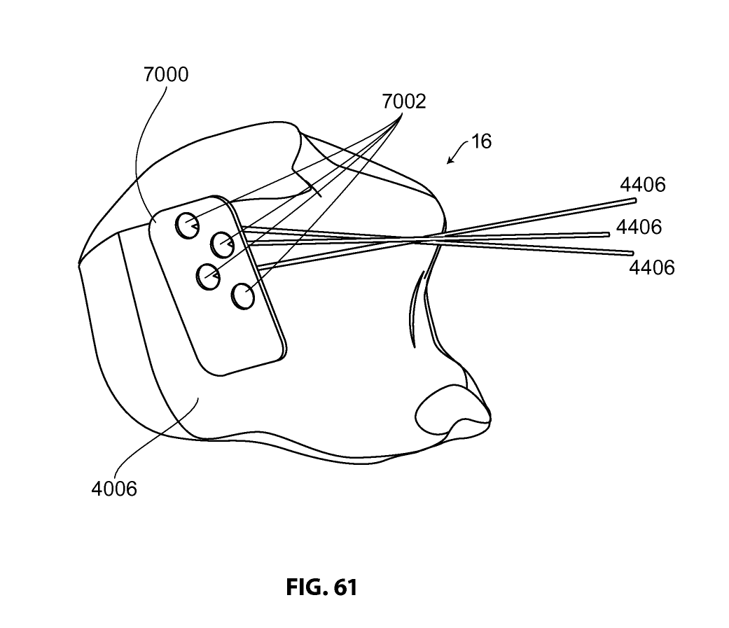

FIG. 61 illustrates an example of a tongue delivery template with position indicators 7002. As shown, a template 7000 includes a template body having one or more position indicators 7002. In some embodiments, the position indicators 7002 may be holes or openings through a thickness of the template body. The holes or openings may extend through the entire thickness of the template body. In some variations, the position indicators 7002 are arranged to correspond to predetermined locations for implants in the patient's tongue. FIG. 61 shows the template body having four openings or position indicators 7002. Two of the indicators are vertically aligned on a left side of the template while two of the indicators are vertically aligned on the right side of the template. The indicators 7002, as shown, are horizontally offset.

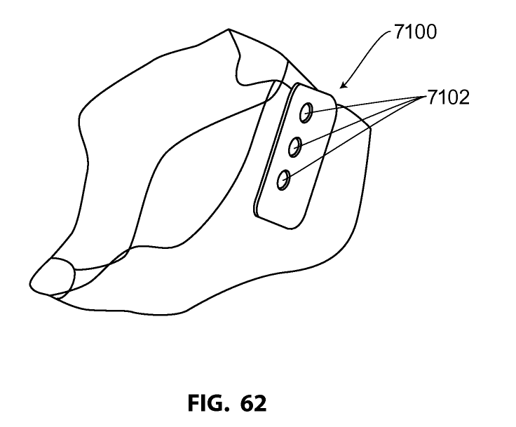

Although shown with four position indicators 7002, the template may include any number of position indicators positioned in any suitable arrangement depending on the desired corresponding implant positions. FIG. 62 shows a three-position indicator embodiment. Moreover, the position indicators 7102 of template 7100 are vertically aligned as shown.

In operation, the physician may affix, adhere, couple, attach, or otherwise place the template onto a surface near or at an airway forming tissue. As shown in FIG. 61, the template 7000 is adhered to the base 4006 of the patient's tongue 16. Once attached, the physician may then begin inserting delivery tools such as wires 4406 or an implant introducer into the patient's tongue. As the delivery tools are inserted, the physician may place a hand onto the tongue with one or more fingers touching at least one of the position indicators 7002. If the delivery tool has been inserted to an adequate tissue depth and proper position, the physician will be able to feel the delivery tool through the template opening 7002 against the base 4006 of the tongue. For example, the physician may feel the tip of a wire 4406 under the tongue base 4006 at one of the position indicators 7002.

In some cases, once the physician receives tactile confirmation of proper delivery tool placement, the physician may continue with implant deployment. If a trocar is used, the physician may insert the trocar into the tongue such that the physician can feel the trocar tip, through tissue, near or at a position indicator on a template. The physician may then push an implant through the trocar to deploy the implant near the trocar tip. The physician may tactilely confirm the deployed implant position by, again, touching the tissue area around, at, or near the position indicator to feel for the implant under the tissue.

The template may be made from any suitable materials with any suitable dimensions. Suitable materials may include those containing silicone or neoprene. The template may be made from a flexible material that can conform to an anatomical shape or surface, such as the surface of the tongue. In some cases, the template material may have a thickness between about 0.010 inches to about 0.040 inches. In other variations, the template may have thickness of about 0.010 inches, about 0.020 inches, or about 0.031 inches. In further variations, the template may have an adhesive backing or a backing/surface adapted to be adhered to tissue. In some embodiments, the template material may have a hardness of about 10 A, about 20 A, about 30 A, about 35 A, and/or about 40 A.

In an alternative variation, the template may be worn on the physician's finger such as a finger template. A finger template may be, for example, a finger cot having position indicators such as a hole or opening on the finger cot. When the physician places his finger against the base of the tongue, tactile feedback can be achieved by feeling for a delivery tool at or near the position indicator.

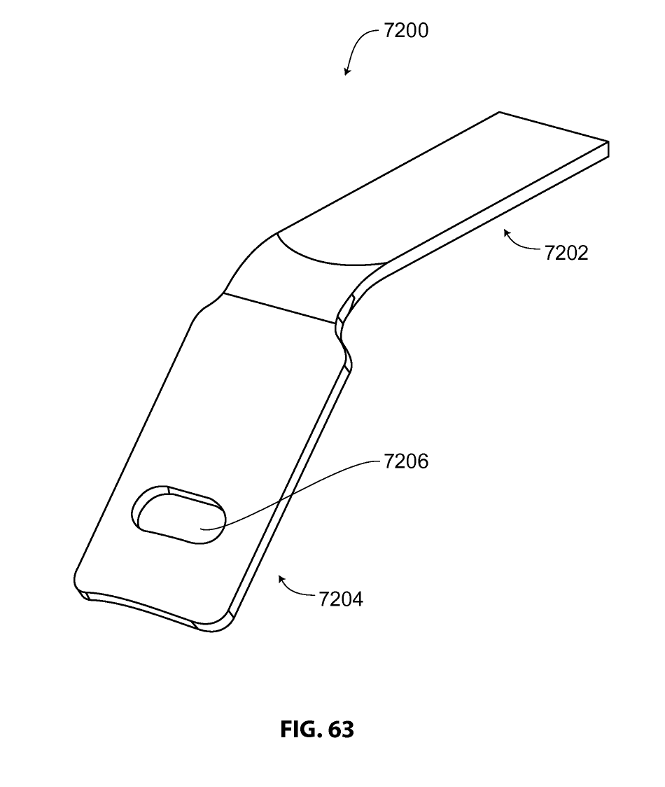

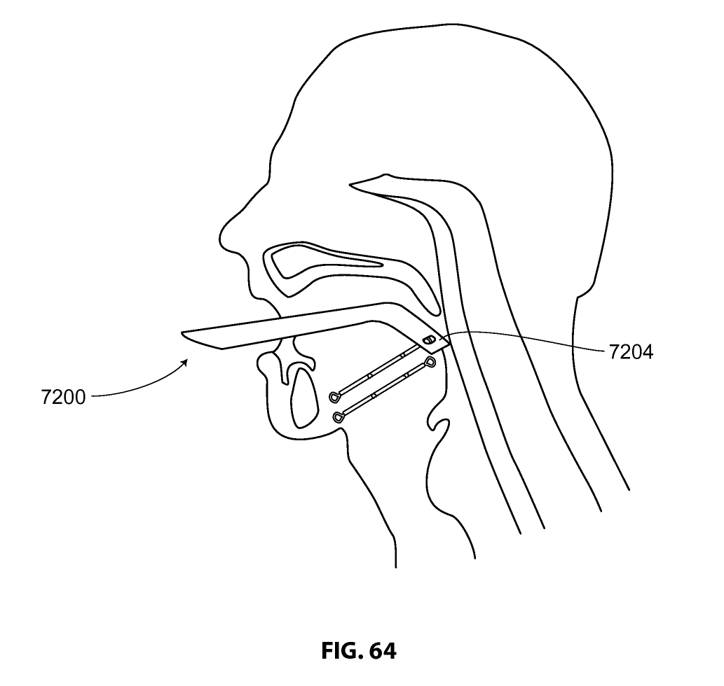

FIG. 63 shows another delivery tool that may be used to aid implant delivery into the patient's tongue. A tongue plate 7200 may include a plate body have a first portion 7202 and a second portion 7204. The first and second portions 7202, 7204 may be adapted to be placed against a surface of the tongue. In some cases, the second portion 7204 is adapted to be placed on a base of the tongue while the first portion 7202 is adapted to be placed on the tongue anterior and superior to the tongue base. As shown in FIG. 63, the plate 7200 may be curved, bent, or angled to fit over the tongue body from the apex to the base. FIG. 64 shows the curved plate 7200 in the patient's mouth on the tongue, where the second portion 7204 is placed against or adjacent the base of the tongue. Additionally, the plate may include a position indicator 7206 like those described with respect to the tissue templates. The position indicator may be a hole through which the physician can use his finger(s) to feel for proper placement of a delivery tool or the implant in the patient's tongue.

In further embodiments, the implant delivery system may include a sheath and dilator assembly. In some embodiments, a dilator is needed to further dilate a delivery path through the patient's tissue in order to accommodate the insertion of the implant. A dilator is typically inserted into a sheath and initially used as a dilator and sheath assembly. The assembly is inserted into the tissue over a wire (although insertion over a wire not required as the sheath with dilator could be directly inserted with the use of a guide assembly similar to the previously described wire guide which uses the outside diameter of the sheath as the determining factor for the channel diameter or width). The wire guide assembly could further be designed with some channels which are to guide wire placement while other channels are for direct sheath insertion without the use of a wire. It is contemplated that any combination of wire and sheath channels could be used in a wire guide. The dilator may dilate or expand the insertion opening and/or the implant delivery path through the tissue to the target implantation site. After the opening and/or tissue delivery path is dilated, the dilator may be removed from the patient by removing the dilator proximally from the sheath. The sheath may remain in the delivery pathway to accommodate insertion of the implant through the sheath and into the target tissue treatment site. After the implant is inserted into the target position in the tissue, the sheath is removed from the patient.

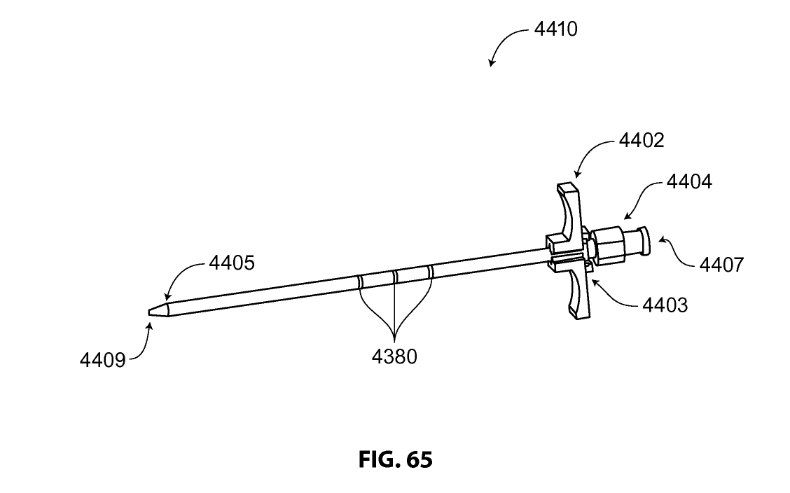

FIGS. 65-67 show a dilator and sheath assembly 4410 that may be used to deliver one or more implants to a patient's airway forming tissue. As shown, the assembly 4410 includes a sheath 4402 having a proximal end 4403 and a distal end 4405. An inner sheath lumen or hollow passageway extends through the sheath body between the proximal and distal ends 4403, 4405. The sheath body may include markings 4380 that provide a visual cue or guide on the assembly's depth of tissue penetration. In some implementations, there are three markings 4380 on the sheath body 4402, corresponding to tissue penetration depths of 55, 65 and 75 mm, respectively. By observing the marking 4380 closest to the entry point through the skin after the sheath assembly has been inserted there through, the appropriate length implant can be chosen (i.e. either a 55, 65 or 75 mm long implant).

The assembly 4410 includes a dilator 4404 received within the sheath 4402. The dilator 4404 extends through the sheath lumen and may extend beyond the proximal and/or distal ends of the sheath. The dilator 4404 also includes a proximal end 4407 and a distal end 4409 with a dilator lumen extending between the ends. The distal end of the dilator 4404 may include a tip portion 5000 and a tip 5001. As shown in FIG. 65, a portion of the dilator's distal end 4409 may extend distally beyond the sheath distal end 4405. The dilator may have a tapered, beveled, or narrowed tip portion 5000 compared to a dilator shaft portion proximal of the tip. In some cases, the length of the tip portion 5000 is about 3 mm to about 4 mm as measured proximally from the distal end 4409. The length between the sheath distal end 4405 and the dilator distal end 4409 may be about 3 mm to about 4 mm.

Any appropriate sizing or dimensions may be applicable for the assembly 4410. The size of the sheath assembly may be determined by the dimensions needed inside sheath diameter to facilitate delivery of the implant. In some cases, the assembly 4410 has a diameter of about 8 Fr. In other embodiments, the assembly has a diameter of about 2 to 12 Fr., about 4 to 10 Fr, or about 6 to 9 Fr.

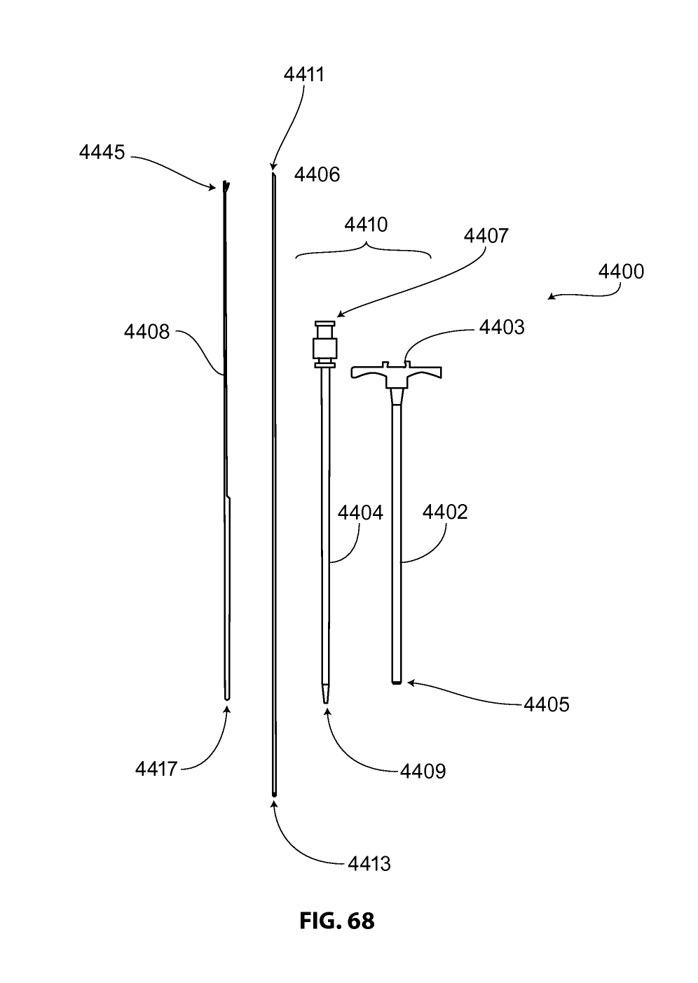

FIG. 68 depicts an example of an implant delivery system including both a wire 4406 and a sheath and dilator assembly 4410. In operation, the wire (e.g. stylet) 4406 may be any of those described for defining a delivery pathway into airway forming tissue. Additionally, the wire 4406 or an axis of the wire may define an implant location in the patient's tissue. In some cases, once a wire is inserted into the tissue, the sheath and dilator assembly may be passed over the wire to further facilitate delivery of an implant to a target implant position in the tissue. In some cases, a plurality of wires may be used to define multiple implant locations in the tissue. The sheath and dilator assembly may be passed over each of the wires to deliver respective implants at each position.

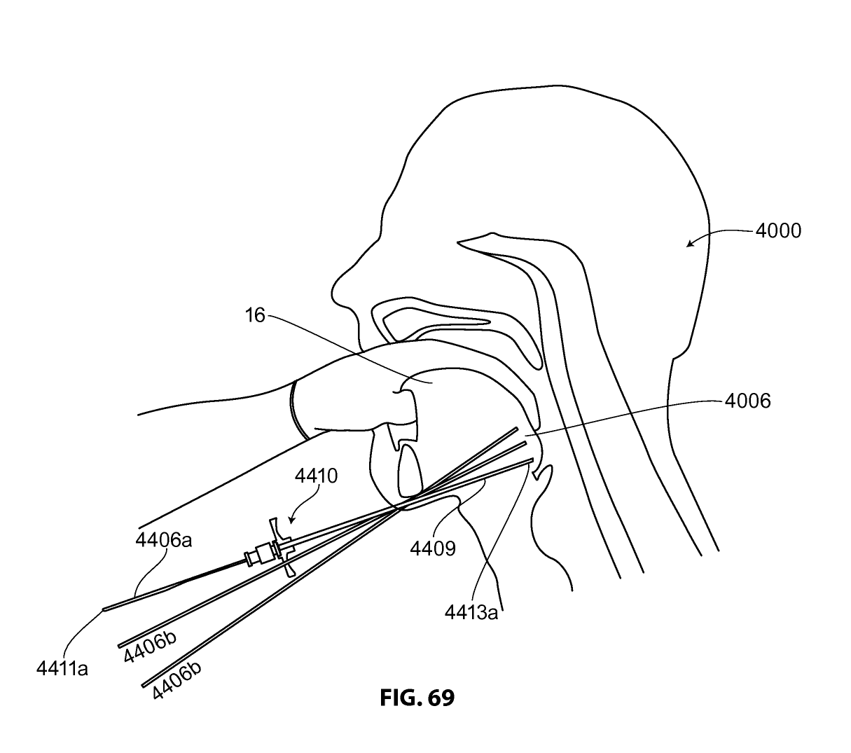

FIGS. 69-74 show an example of such a delivery using a plurality of wires defining multiple implant positions in the tongue. In the depicted procedure, three implants 4100 will be delivered to three different implant positions within the patient's tongue. FIG. 69 shows the respective orientation and position of a first, second and third wire 4406a-c in the patient. The wires 4406a-c each includes a proximal portion that resides outside of the patient, which corresponds to the position of each wire relative to the others in the tissue. The wire positions also define the desired position and orientation of three implants in the target tissue site. Once the first, second, and third wires 4406a-c are in place, the sheath and dilator assembly 4410 may be passed over each wire separately.

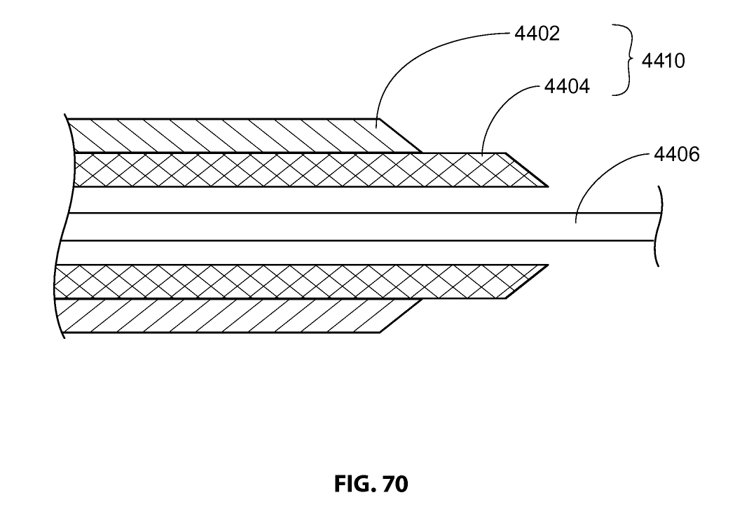

FIG. 69 shows the assembly 4410 passing over the first wire 4406a. The first 4406a is received within the dilator lumen as the assembly 4410 is passed over the wire 4406a and through the tissue. The assembly 4410 may be introduced and inserted partially into the patient such that the distal end 4409 of the dilator 4404 is near or at the distal end 4413a of the first wire 4406a. FIG. 70 shows a cross-sectional view of the delivery system where the assembly 4410 is passed over the wire (e.g. stylet) 4406.

After the assembly 4410 is in the desired position within the tissue, the sheath 4402 may be further advanced over the dilator 4404 to align the dilator distal end 4409 with the sheath distal end 4405. The sheath may be moved over the dilator 4404 body such that the sheath distal end 4405 overlaps or co-extends with the dilator tip 5001. This can ensure that the distal end 4405 of the sheath 4402 is in the proper position for implant deployment.

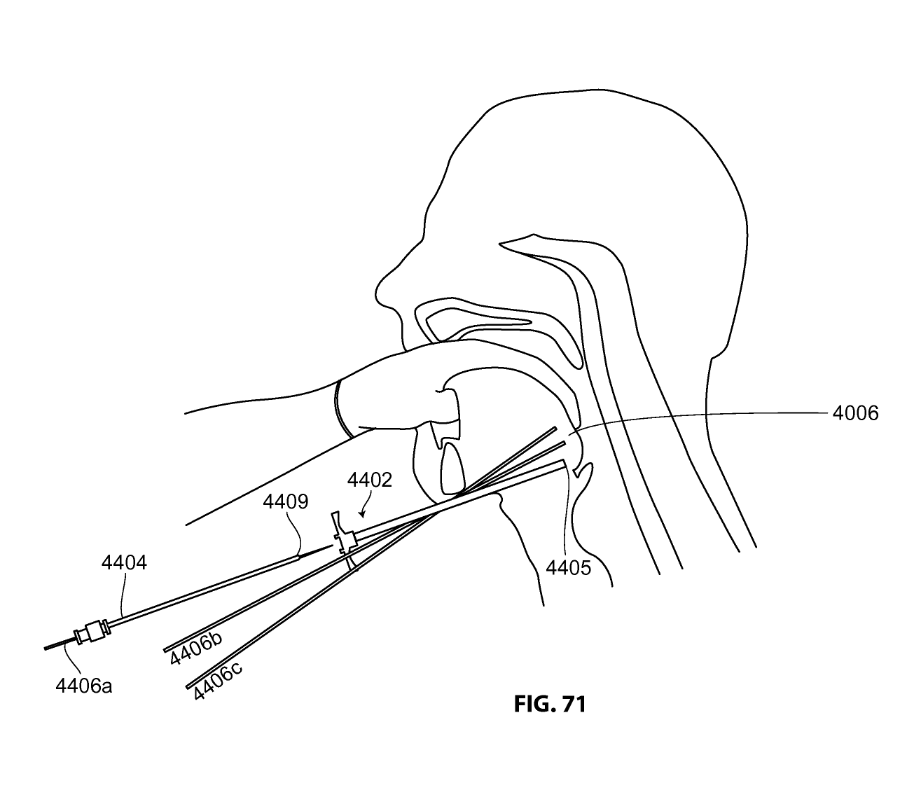

As shown in FIG. 71, the dilator 4404 may be removed from the sheath 4402 by proximally pulling the dilator 4404 out of the sheath lumen. In some cases, the assembly 4410 may include a locking mechanism configured to releasably retain the dilator in the sheath. Releasing the locking mechanisms allows the dilator to be removed from the sheath.

Where a wire is used, the wire may also be removed from the patient once the assembly 4410 is in place. FIG. 71 shows the first wire 4406a and the dilator 4404 are both removed from the incision site.

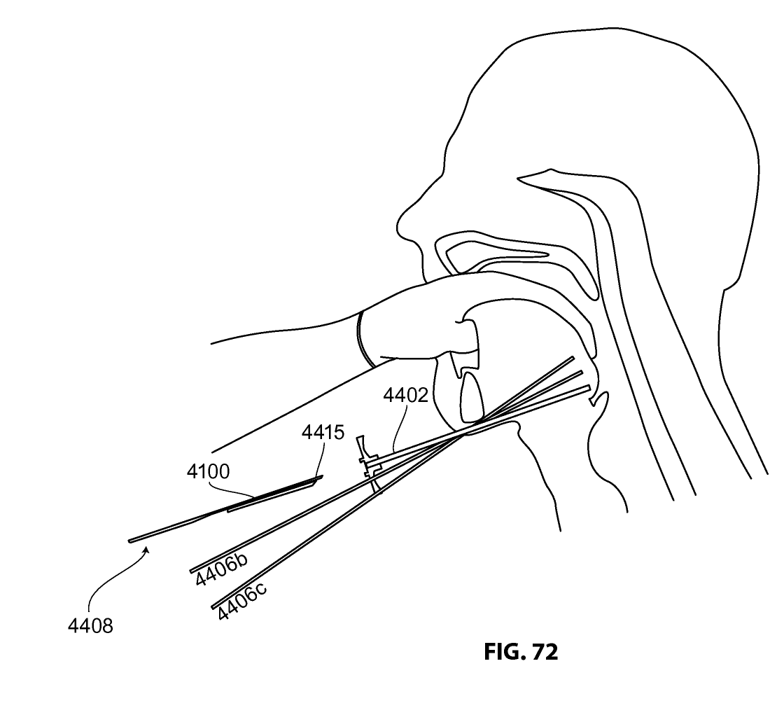

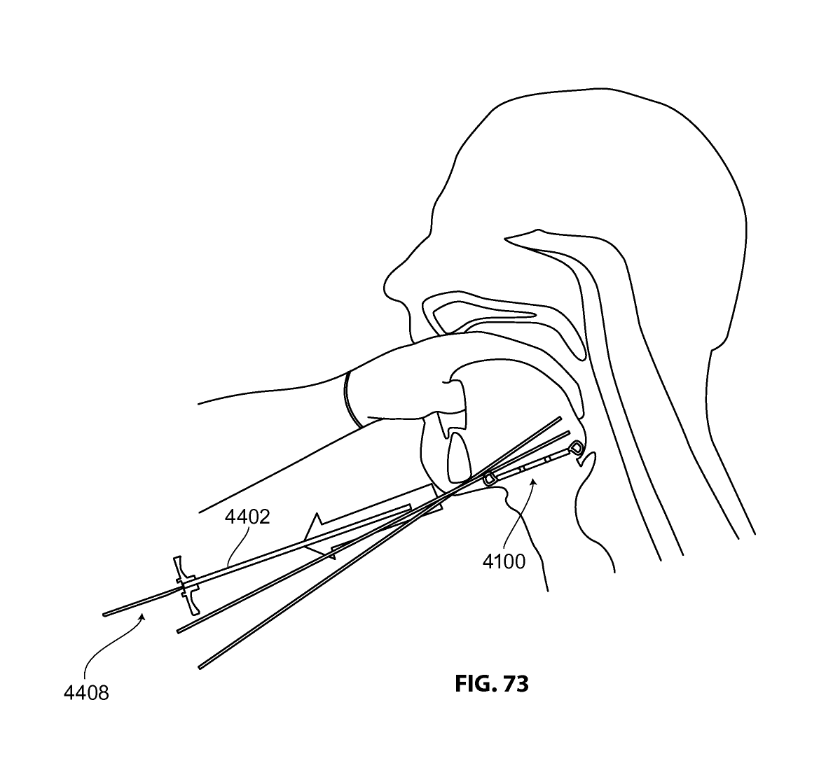

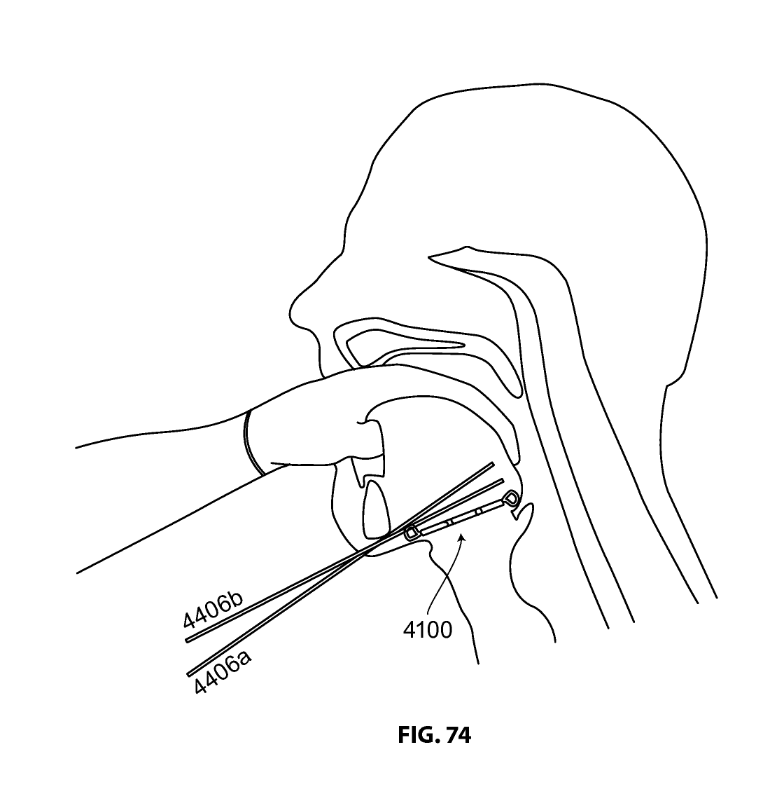

To deploy the implant in the target tissue site, the implant may be introduced by a push rod as described above. The push rod 4408 may having a proximal end 4417 and a distal end 4415 adapted to engage an implant. The push rod 4408 may engage the implant 4100 such as by engaging the distal end of the implant 4100. The push rod 4408 is adapted to deliver the implant 4100 through the sheath lumen. In some cases, a distal end of the implant 4100 is deployed in the tissue at or near the distal end 4405 of the sheath (shown in FIG. 72 near the base of the tongue). Once the implant is deployed, the push rod and sheath are removed from the patient (FIG. 73). After the first implant has been delivered, the remaining second and third wires 4406b-c (shown in FIG. 74) each provides a delivery path for the remaining implants. The same or different sheath and dilator assembly may be advanced over the remaining wires 4406b-c to deliver implants to the target implant positions in the patient's tongue.

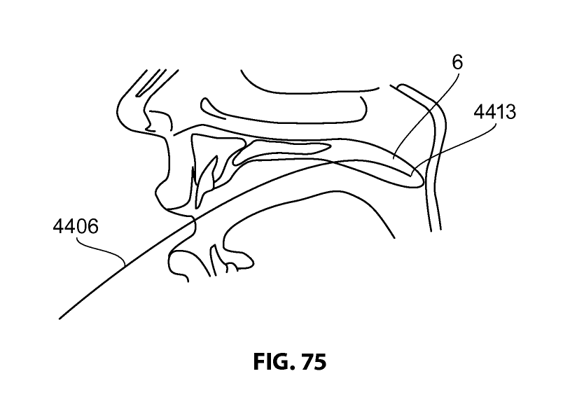

FIG. 75 depicts a similar implant delivery procedure for the soft palate. As shown, the wire can be introduced into the soft palate through an incision. The wire may be partially introduced into the patient's mouth and partially inserted through the incision. The wire may define a delivery path for delivering an implant into the tissue and an implant position in the tissue. For example, the distal end 4413 of the wire 4406 may correspond with a distal end of the implant in situ. Likewise, the slope or angle of the wire 4406 may define the angle of the implant in the soft palate.

In some cases, the wire may be sufficiently flexible to bend or curve to accommodate curvature in the target tissue area. For example, the wire 4406 may include a portion that bends in a curved area of the soft palate. As such, this can avoid using a stiff wire that does not bend and would instead perforate straight through the curved area of the soft palate 6.

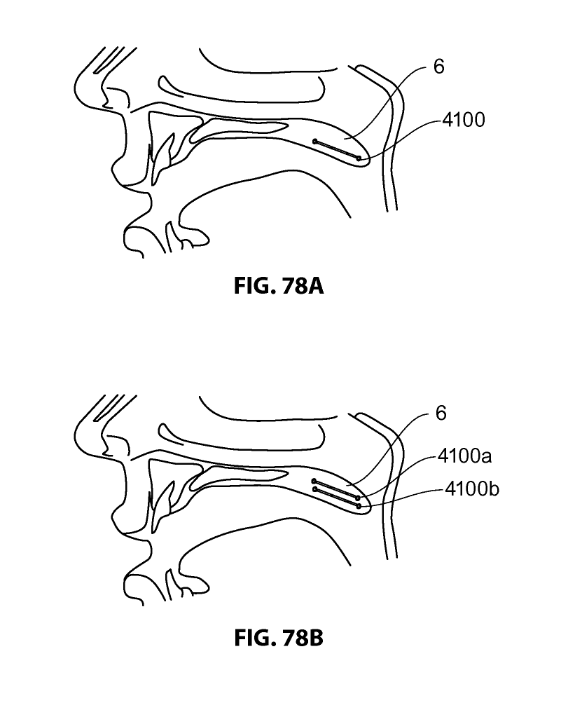

Once one or more wires have been placed in the soft palate 6, a sheath and dilator assembly may be introduced over the wire as described in an earlier section (FIGS. 75-77). The dilator may be removed with the wire to leave a remaining sheath in position for receiving an implant through a sheath lumen. The implant may be deployed in the soft palate by using a push rod (as described) to move the implant through the sheath and into the target tissue site. These steps may be repeated for additionally implants. Another wire may already be in place for use of the sheath and dilator assembly in deploying a second implant in the soft palate (FIGS. 78A-B).

Multiple implants may be placed in the soft palate. The implants may include any suitable orientation relative to one another. In some cases, the implants are separated by at least about 0.25 cm, at least about 0.50 cm, at least about 1 cm or at least about 1.5 cm. In one variation, the spacing is at least about 0.50 cm.

In other cases, the implants are separated by an angle between about 0 degrees to about 60 degrees. In some variations, the implants form an angle between about 0 degrees to about 45 degrees. In further embodiments, the angle is between about 10 degrees to about 30 degrees. In additional embodiments, the angle is between about 15 degrees to about 45 degrees. As used herein, the angle may be the angle between two or more implants.

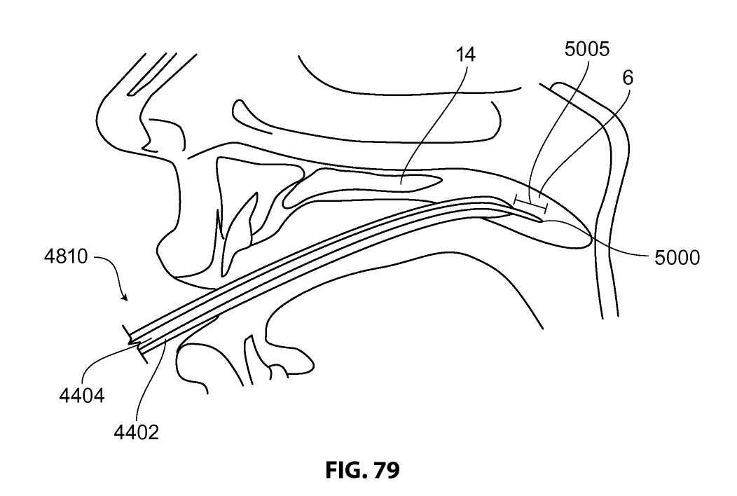

In some embodiments, the sheath and dilator assembly can be used to deliver an implant without a wire, stylet, guidewire, etc. defining the delivery path prior to insertion of the assembly. In such cases, it may be advantageous to include a dilator tip configured to steer the assembly along a path of the tissue without perforating or coring through the dermal or mucosal lining of the tissue. FIG. 79 shows an assembly 4810 having a dilator 4804 in a sheath 4802. The distal end 4809 of the dilator 4804 includes a tip portion 5005 that extends distally beyond the distal end 4805 of the sheath. The tip portion 5005 includes an eccentric tip 5003. As shown, the eccentric tip 5003 helps to steer or direct the dilator tip portion 5005 along the curved portion of the soft palate 6 such that the tip 5003 does not dissect through the lining of the soft palate and out of the tissue.

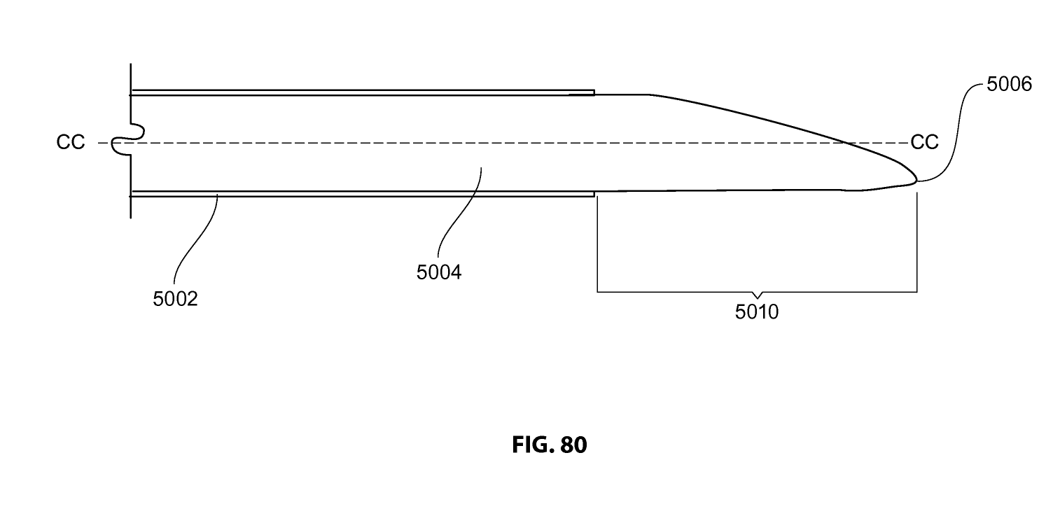

FIGS. 80-85 depict examples of eccentric dilator tips that facilitate the insertion of the sheath and dilator assembly into curved tissue without perforating through the tissue lining. FIG. 80 shows a dilator 5004 in a sheath 5002. A dilator tip portion 5010 with a tip 5006 extends beyond the distal end of the sheath 5002. The dilator body includes a central longitudinal axis CC that extends lengthwise through the center of the dilator 5004. The eccentric tip 5006 is offset from the central longitudinal axis CC.

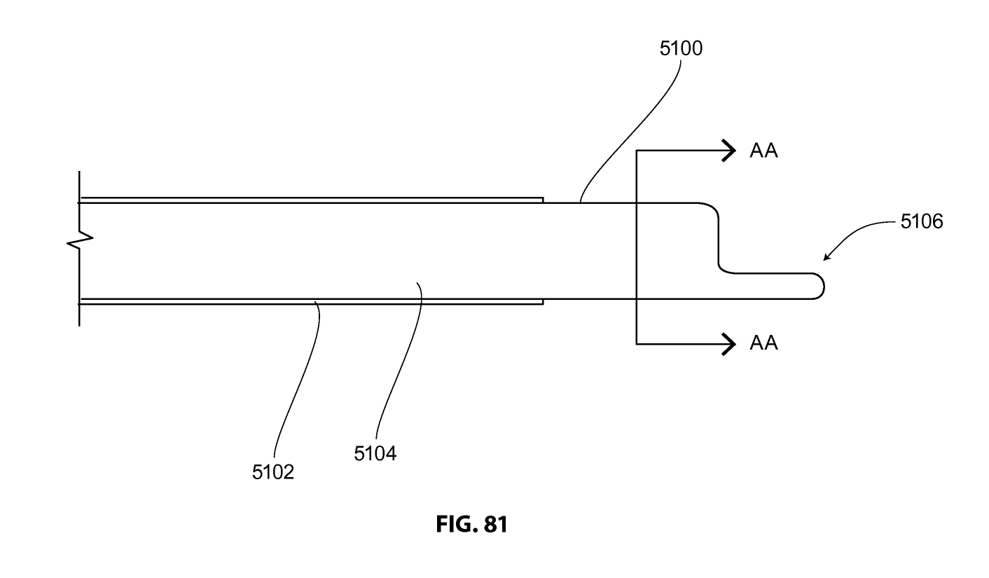

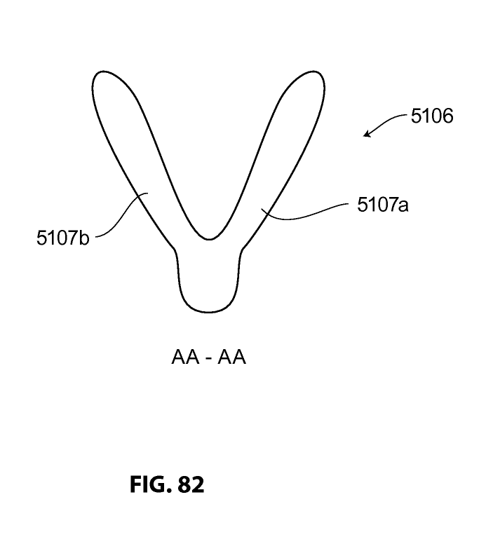

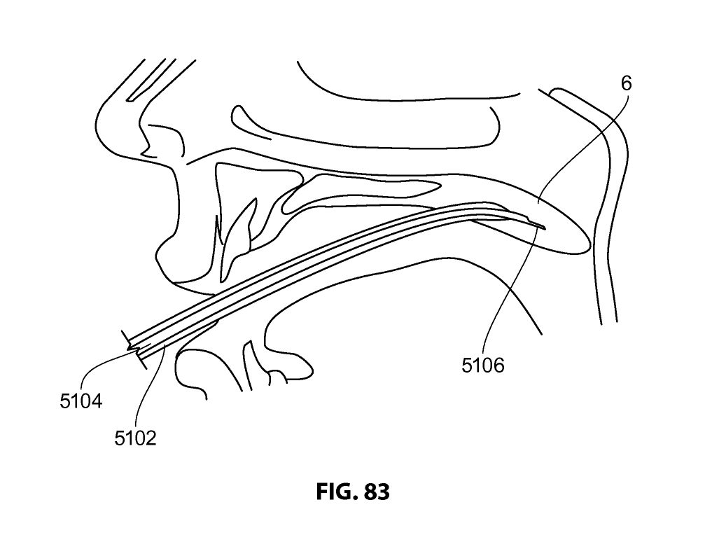

Similarly, FIG. 81 depicts another example of an eccentric dilator tip. The dilator 5104 resides within the sheath 5102 with a tip portion 5100 extending distally from the sheath. The tip portion 5100 includes an eccentric tip 5106 having a reduced cross-section relative to the dilator body. The cross-section, as shown along line AA in FIG. 82, of the eccentric tip 5106 and/or tip portion 5100 may include a wedged or V shape. In some cases, the tip portion 5100 may include lobes 5107a-b. As shown in FIG. 83, the eccentric dilator tip may be configured to move the assembly toward the centerline of the soft palate. This may ensure that the tip of the assembly does not cut through the soft palate.

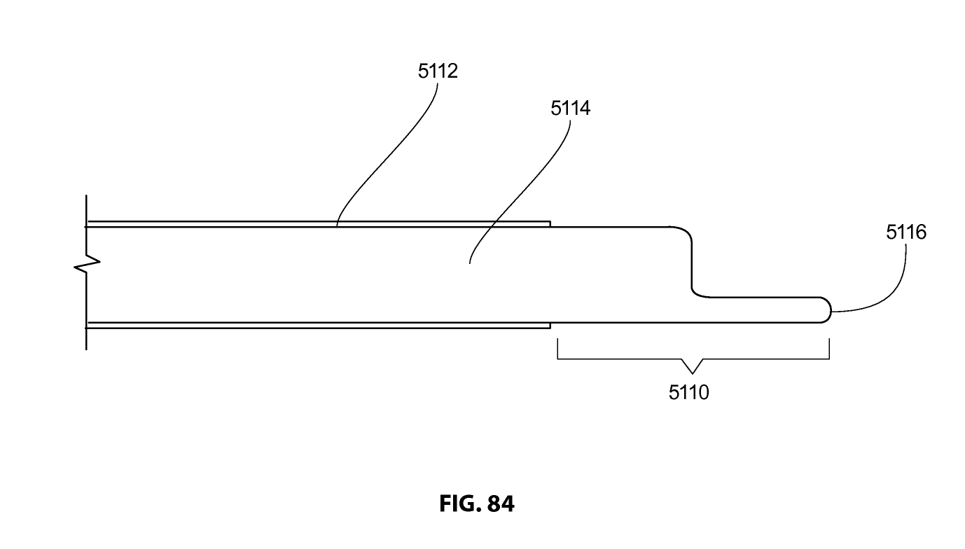

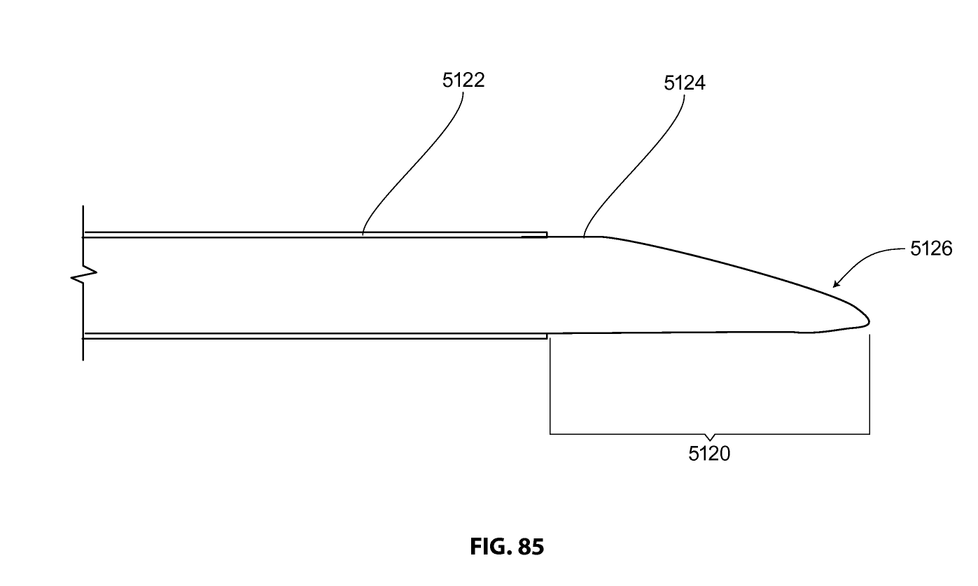

FIGS. 84-85 show additional variations of the sheath and dilator assembly with an eccentric tip. FIG. 84 shows a sheath 5112 and a dilator 5114 with a dilator tip portion 5110. The tip portion 5110 includes a tip 5116 having a smaller cross-section compared to another section of the tip portion 5110. FIG. 85 shows a sheath 5122 extending around a dilator 5124 with a tip portion 5120 having a tapered eccentric tip 5126.

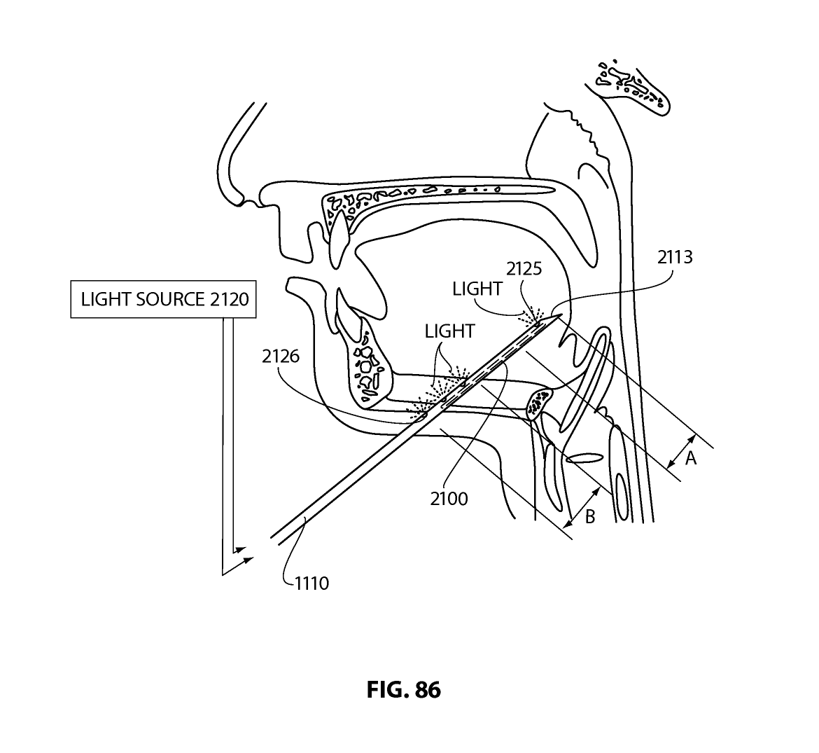

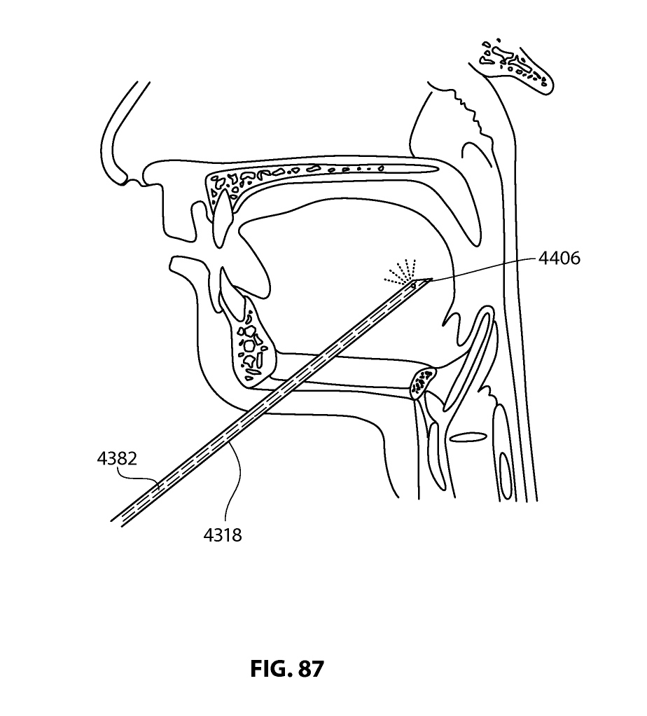

Referring to FIGS. 86-88, other contemplated embodiments provide for light guided or assisted implant delivery. Referring to FIG. 86, it can be seen that an elongate wire 2110 includes a light source 2120 that is coupled to a light emitter 2125 carried at a distal end 2113 of the wire. The light source can be any non-coherent or coherent light in wavelength(s) that will be visible by the physician during the implantation procedure. In use, the physician can observe the light as the wire penetrates closer to the surface of the tongue, and thus can determine the optimal insertion location of the distal end 2113 of the wire, which may correspond to the distal end of an implant once the implant is deployed in the target tissue site. In FIG. 86, it can be further seen that the wire body may include additional light emitters 2126 along its medial and proximal regions, which can be used to determine the penetration depth when the physician has used the light emission to optimize the location of the distal end 2113 of the wire 2110. In some cases, referring to FIG. 87, the wire may comprise a hollow lumen or channel 4381 for directing light through the wire to the distal end 2113. The light emission can be provided by light propagating in a light channel extending to the working end, or from an LED carried by the working end. The channel may receive or include a light guide 4382 that can be coupled to a remote light source. The light guide may be adapted to allow light propagation therethrough by internal reflection in the light guide region and then outward light emission by the reflective material.

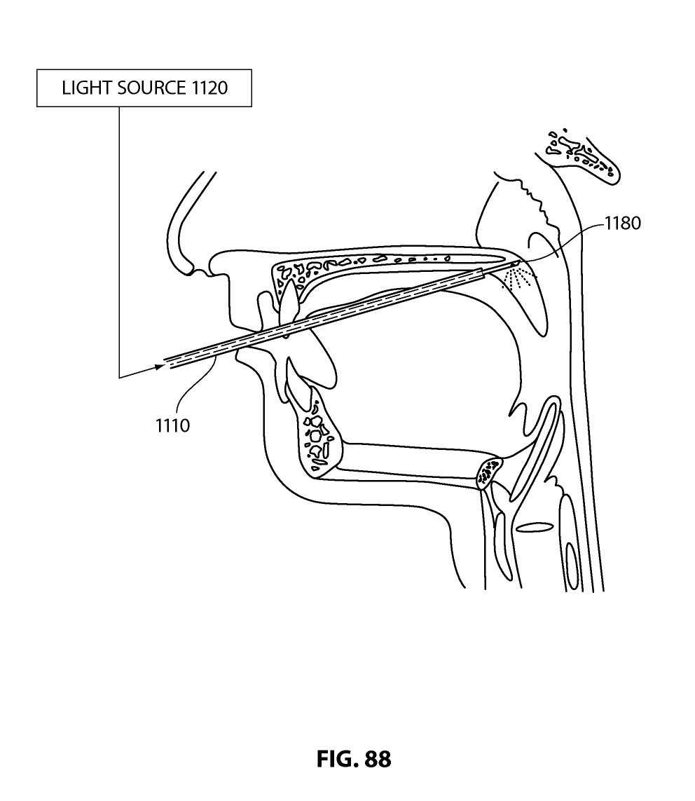

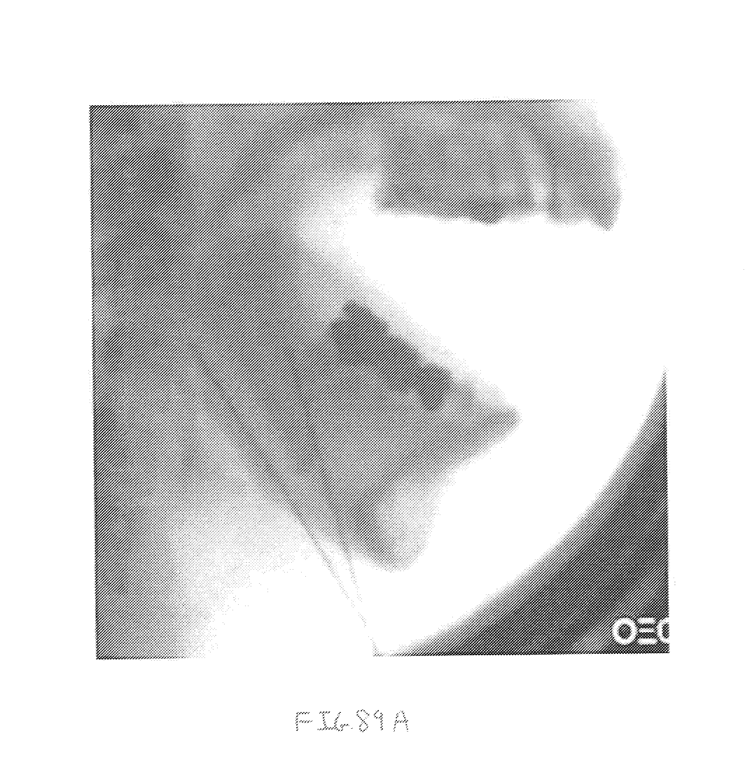

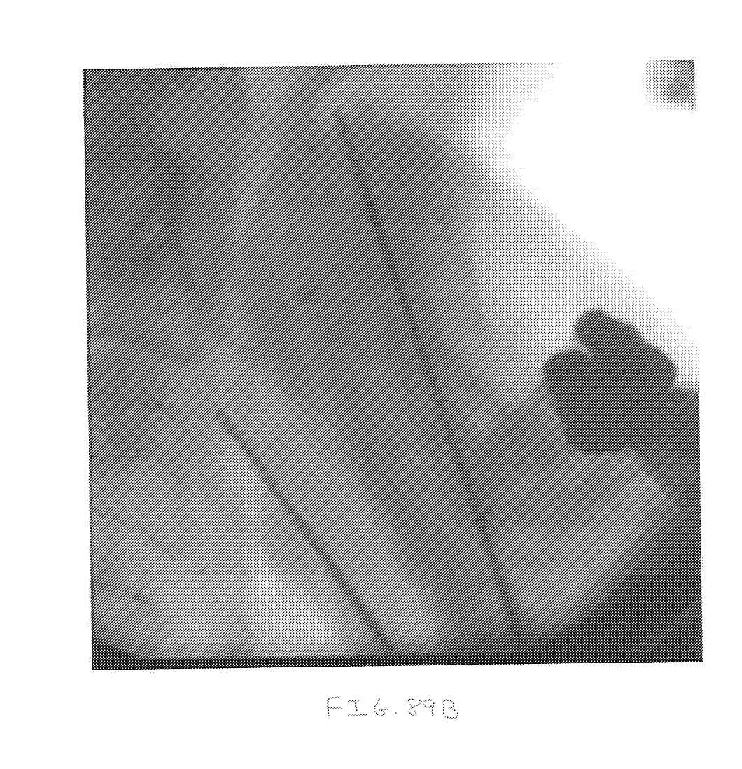

FIG. 88 represents an introducer system, such as a sheath and dilator assembly, that includes light emission and guidance. In this embodiment, the assembly includes one or more light emitters 1125 on a distal, medial, or proximal region of the assembly. The emitters may be axially spaced apart in a manner that will assist the physician in determining the depth of penetration by the assembly. In some cases, the physician maneuvers the assembly through the patient tissue with the goal of avoid excessive penetration. If the physician sees the light through the tissue, this can be an indication of excessive depth penetration. For example, the soft palate is a relative thin tissue with a typical thickness of about 6 cm to about 7 cm. This makes it relatively easy to drive an assembly tip through the lining of the soft palate, which tears and injures the tissue. As such, the light emission can serve as a visual warning that the tip is close to the outer surface of the soft palate.

In any of the described embodiments, the light emitters can range in number from two to ten or more and be spaced apart by a dimension of 1 mm to 10 mm. A controller and switching mechanism may be provided to activate the light emitters one at a time or in sequence. Also, the light emitter can provide different wavelength and thus different visible colors to assist in determining the location of each light emitter in the tissue. Alternatively, the light can be emitted through colored lenses to provide a plurality of colored light emissions.

In general, the term light emitter as used herein includes a remote light source coupled to a light guide in the introducer, wherein the light guide can comprise an optic fiber or other channel with light emission from the distal end of the channel. A plurality of emitters can be coupled to a plurality of light guides or a single light guide can have a plurality of light emitting points, for example light emission regions along the length of an optic fiber. In one embodiment, an optic fiber is carried in the wall of the introducer sleeve. In any embodiment, the light emitter also can comprise an LED or similar light emission source disposed on the introducer that is coupled to a power source.

### G. Methods of Implantation

As described, placing multiple implants in a patient may provide better tongue or other tissue remodeling, better tongue or other tissue control, fewer side effects and/or may allow smaller implants to be placed. Multiple incisions may be made and used to place implant(s) or two or more implants may be placed through a single incision. Another method of implanting an implant or treating a treating an airway disorder or otherwise treating airway, mouth, nasal, or throat tissue may include creating a surface incision on a surface of a tissue near an airway forming tissue, placing a delivery device holding a first elongate implant at least partially through the incision and into the airway forming tissue, placing the first elongate implant into a first position in the airway forming tissue, removing the delivery device from the airway forming tissue wherein the first elongate implant remains in the airway forming tissue, placing a second delivery device holding a second elongate implant through the incision and into the airway forming tissue, placing the second elongate implant into a second position in the airway forming tissue, and removing the second delivery device from the airway forming tissue wherein the second elongate implant remains in the airway forming tissue. A surface incision may be any size required but preferably is very small. An incision may be less than 3 cm, less than 2.5 cm, less than 2 cm, less than 1.5 cm, less than 1 cm, or less than 0.5 cm in a widest dimension. Placing the first implant may include placing it on one side of a midline of a tongue and placing the second implant may include placing it on the other side of the midline of the tongue.

If the first implant has a first axis forming a first angle with a transverse plane of the patient and the second implant has a second axis forming a second angle with the transverse plane of the patient, placing the first and second implants may include forming oblique angles between the first and second axes and the transverse plane. If the first implant has a first axis forming a first angle with a midline plane of the tongue and the second implant has a second axis forming a second angle with the midline plane of the tongue, wherein placing the first and second implants comprises placing each implant axis at an angle oblique to the midline plane. In some embodiments, the same delivery device may be used to place the first and second (or more) implants. In some embodiments, different delivery devices may be used to place the first and second (or more) implants.

In general, a method for treating an airway disorder comprises implanting an implant body into airway-interface tissue wherein the implant body is sized and shaped to conform in a manner compatible with normal physiological function of the site and to apply selected forces to the tissue, and wherein the implant is configured to receive an electromagnetic query and to respond with an electromagnetic signal indicating an operational parameter of the implant body during said normal physiological function of the site.

The embodiments of implants shown in the figures above can be sized and shaped to conform to a treatment site in a patient's tongue, palate or other site in airway-interface tissue and to reside in an orientation and in a manner compatible with normal physiological function of the site. The overall dimensions may vary according to the full extent that human subjects vary in their anatomical dimensions, and thus the dimensions provided here are only an approximation for the purpose of illustration, and are not meant to be limiting. Any embodiment in its elongated state may typically be in the range of about 2 cm to about 10 cm in length in a releasably extended state, and the implant in a contracted state may be in the range of about 1 cm to about 6 cm in length. Testing shows there is an advantage to using these lengths.

In other embodiments, a method of treating an airway disorder may include delivering one or more implants into a target tissue site using at least one wire. In some cases, the method of treatment may including the steps of creating an incision on a surface of a tissue near an airway forming tissue, partially inserting at least a first wire and a second wire into the airway forming tissue through the incision, wherein a first axis of the first wire defines a first implant position for a first implant in the airway forming tissue and a second axis of the second wire defines a second implant position for the second implant in the airway forming tissue, the first axis and the second axis forming an angle between about 0 degrees to about 45 degrees, placing a first implant at the first position in the airway forming tissue by guiding the first implant to the first implant position along a first path defined by the first axis of the first wire, placing a second implant at the second position in the airway forming tissue by guiding the second implant along a second path defined by the second axis of the second wire, and removing the first and second wires from the airway forming tissue.

As described, the one or more wires may be partially inserted into the tissue and adjusted to independently define an implant position within the target tissue site. The first wire may include an axis that defines the orientation of a first implant position relative to a second implant position that is defined by another axis of the second wire. The distal ends each wire may correspond with the position of a respective end for each corresponding implant. The distal end of the first wire may correspond with the distal end of the first implant.

In some cases, after the first and second wires have been partially inserted into the tissue, the physician may adjust the position of the two wires relative to each other to achieve a desired displacement spacing, orientation, and/or angle between the wires. For example, the physician may reduce or increase an angle formed between the wires. The physician may also vertically or horizontally offset the wires from one another. Additionally, the physician may adjust the relative depth of penetration between the wires where the distal ends of the wires do not have the same depth of penetration into the tissue. In some cases, the physician may partially insert the first and second wired into tissue to determine the positioning of the relative wire angles in tissue without the use of his hand or finger in the patient's mouth as shown in FIGS. 46A, 59 and 71. The physician may then place his hand or fingers in the patients to complete the depth of insertion of the wires using tactile feedback to precisely determine the depth of insertion. This method allows for the near complete placement and determination of the relative angles without the hand or fingers in the mouth which could distort the tongue position. This minimizes the chance for placement variability due to movement of the tongue from its natural position while allowing the use of the hand or fingers for precise wire depth insertion. As can be appreciated, any variation of positioning may be achieved by adjusting the wires to result in corresponding desired implant positions optimal for therapeutic effect.

As non-limiting examples, in some embodiments, the first and second implant positions are spaced apart by at least about 0.25 cm or about 0.50 cm as described in an earlier section. In further variations, the first and second implant positions form an angle between about 0 degrees to about 60 degrees or between about 0 degrees to about 45 degrees. In further embodiments, the angle is between about 10 degrees to about 30 degrees. In additional embodiments, the angle is between about 15 degrees to about 45 degrees. As used herein, the angle may be the angle between two or more implants. Likewise, the angle formed between implant positions may be an angle between two implant positions or a total angle (e.g. sum) of the angles between multiple implant positions.

Where the treatment includes more than two implants, the method of treatment may include the steps of partially inserting at least a third wire and a fourth wire into the airway forming tissue through the surface incision, the third wire having a third axis that defines a third implant position for a third implant in the airway forming tissue and the fourth wire having a fourth axis defining a fourth implant position for the fourth implant in the airway forming tissue, wherein a total angle formed by the first, second, third, and fourth axes is between about 0 degrees to about 45 degrees, placing a third implant at the third position in the airway forming tissue by guiding the third implant to the third implant position along a third path defined by the third axis of the third wire, placing a fourth implant at the fourth position in the airway forming tissue by guiding the fourth implant along a fourth path defined by the fourth axis of the fourth wire, and removing the third and fourth wires from the airway forming tissue.

In some cases, the wires may be aligned, positioned, placed, or orientated with a guide tool or template. As such, in some cases, the method of treatment may include the steps of inserting a portion of the first wire into a first wire channel of a wire guide, wherein the portion of the first wire in the first wire channel is laterally engaged by the wire guide, and inserting a portion of the second wire into a second wire channel of the wire guide, wherein the second wire channel defines an alignment between the second wire and the first wire in the airway forming tissue, the alignment comprising an angle between the second wire and the first wire. In some embodiments, the angle may be between about 0 degrees and about 45 degrees. In some cases, the wire guide vertically offsets the first and second wires from one another. Additionally, the method may also include releasing at least one of the first or second wires from a wire channel by rotating the wire guide. The releasing step may be accomplished by laterally releasing an inserted wire.

Additionally, the treatment method may include the steps of inserting at least the first and second wires into a wire guide positioned proximal to the incision, wherein the wire guide maintains an orientation of the first axis and the second axis relative to each other, and removing the first and second wires from the wire guide without removing the first and second wires from the incision.

In other embodiments, the treatment method may include inserting the first or second wire into the patient's tissue (e.g. tongue) with less than 2 lbs of force. In some cases, the insertion force is about 1 lb of force. In other embodiments, the insertion force is about 1.2 lbs of force. In further embodiments, the insertion force is about 1.5 lbs or less.

In other cases, the treatment method may include applying a tissue template and/or tissue placement tool to provide the physician with a position guide. The template or tool may allow the physician to feel the position of a wire or other delivery tool tip through tissue. This provides tactile feedback on tissue depth penetration and delivery tool location in the target treatment site.

In another aspect, a method of treating an airway disorder may include the steps of creating an incision on a surface of a tissue near an airway forming tissue, partially inserting a first wire and a second wire into the airway forming tissue through the incision, wherein a first axis of the first wire defines a first implant position for a first implant in the airway forming tissue and a second axis of the second wire defines a second implant position for the second implant in the airway forming tissue, guiding an implant delivery device through the incision and at least partially into the airway forming tissue by advancing the delivery device along a length of the first wire, placing a first implant at the first position in the airway forming tissue, removing the implant delivery device from the airway forming tissue after placing the first implant in the first position, guiding the implant delivery device through the incision and at least partially into the airway forming tissue by advancing the delivery device along a length of the second wire, placing a second implant at the second position in the airway forming tissue, and removing the implant delivery device from the airway forming tissue after placing the second implant in the second position.

In further variations, the treatment method may include releasably engaging an end of the first implant with a pusher; inserting the pusher through a lumen of the implant delivery device to deploy the first implant at the first position, releasably engaging an end of the second implant with a pusher, and inserting the pusher through a lumen of the implant delivery device to deploy the second implant at the second position.

In some embodiments, the implant delivery device is a sheath and dilator assembly, trocar, cannula, or other suitable introducer. The assembly may include a dilator with a lumen configured to receive a wire. The dilator may be configured to advance over the received wire for guiding insertion of the sheath and dilator assembly into the airway forming tissue. In further variations, the treatment may include the steps of removing the dilator from the assembly, pushing the first implant through a lumen in the sheath to deploy the first implant in the first position in the airway forming tissue, and pushing the second implant through a lumen in the sheath to deploy the second implant in the second position in the airway forming tissue.

As described, the dilator may include an eccentric tip. As such, the eccentric tip may be configured to guide the dilator along a curved portion of the airway forming tissue. In some cases, the eccentric tip is configured to guide the dilator along a path near a midline of the airway forming tissue.

In further embodiments, the treatment method may optionally avoid the use of wires. In some cases, the treatment method includes creating an incision on a surface of a tissue near an airway forming tissue, advancing a sheath and dilator assembly through the incision and at least partially into the airway forming tissue, wherein the dilator comprises an eccentric tip configured to guide the assembly along a curved area of the airway forming tissue, placing a first implant at the first position in the airway forming tissue, and placing a second implant at the second position in the airway forming tissue, and removing the assembly from the airway forming tissue. As described, the use of an eccentric tip may accommodate positioning the tip of the dilator near a midline of the airway tissue.

In other embodiments, the treatment method may include inserting the implant delivery device into the patient's tissue (e.g. tongue) with less than 2 lbs of force. In some cases, the insertion force is about 1 lb of force. In other embodiments, the insertion force is about 1.2 lbs of force.

Additionally, light guidance or assistance can be used. In some variations, the treatment method includes emitting light from a portion of a delivery device such as the sheath and dilator assembly and/or wire(s). The method may include detecting the emitted light outside of the airway forming tissue to thereby determine a depth of penetration by the delivery tool in the airway forming tissue.

### EXAMPLES

### Example 1

Bench testing was conducting on a cow tongue specimen to evaluate a variety of stylets, needles, and wires for insertion force, ability to travel smoothly, and ease of perforation. Tested designs are described below in Table 1:

TABLE 1
Insertion testing

| | diameter | |
|---|---|---|
| Equipment tested | (inches) | Tip style |
| Teflon coated wire | 0.040 | very blunt (edges broken by hand) |
| Teflon coated wire | 0.040 | domed (handmade) |
| Teflon coated wire | 0.040 | bevel (handmade) |
| Solid stylet | 0.020 | |
| Solid stylet | 0.024 | sharp (yellow) |
| Solid stylet | 0.037 | sharp (pink) |
| Solid stylet | 0.050 | Shallower bevel than previous stylets |
| | | (gray) |
| Needle | 0.060 | bevel tip |
| Needle | 0.050 | lancet tip |
| Needle + Stylet | 0.060 | bevel tip (with gray stylet) |

Table 2 below qualitatively describes the relative insertion force, smoothness of travel through the cow tongue tissue, and ease of perforation by each of the tested designs. The solid stylet with a shallow bevel tip and the wire with a domed tip appeared to perform better overall in each of the three categories. Additionally, as shown in Table 2, the sharp tipped designs demonstrated a tendency to perforate tissue.

TABLE 2
Insertion testing

| | | | Rating (1 is worst, 3 is best | | | |
|---|---|---|---|---|---|---|
| | | | | Travels | | |
| | | | | Smoothly | Does not | |
| Equipment | diameter | | Start force is | and | perforate | |
| tested | (inches) | Tip style | low | Easily | easily | Notes |
| Teflon | 0.040 | very blunt (edges broken by hand) | 1 | 1 | 3 | |
| coated | | | | | | |
| wire | | | | | | |
| Teflon | 0.040 | domed (handmade) | 2 | 2 | 3 | |
| coated | | | | | | |
| wire | | | | | | |
| Teflon | 0.040 | bevel (handmade) | 2 | 2 | 2 | |
| coated | | | | | | |
| wire | | | | | | |
| Solid stylet | 0.020 | | 3 | 2 | 1 | |
| Solid stylet | 0.024 | sharp (yellow) | 3 | 3 | 1 | |
| Solid stylet | 0.037 | sharp (pink) | 3 | 2.5 | 1 | |
| Solid stylet | 0.050 | Shallower bevel than previous | 2.5 | 2.5 | 2.5 | |
| | | stylets (gray) | | | | |
| Needle | 0.060 | bevel tip | 2.5 | 2 | 2.5 | Cored |
| Needle | 0.050 | lancet tip | 3 | 2 | 1 | Cored |
| | | | | | | a lot |
| Needle + | 0.060 | bevel tip (with gray stylet) | 2.5 | 2.5 | 2 | |
| Stylet | | | | | | |

### Example 2

Bench testing was conducted on a cow tongue to evaluate tissue template sheet materials. Sheet material was compared for ability to adhere to tissue and ability to tactilely differentiate between tissue and sheeting. Table 3 describes the sheet materials tested. For testing, each sheet was placed against the surface of the cow tongue.

TABLE 3
Silicone sheeting Testing

| Durom- | Thick- | | Surface | | Mcmaster |
|---|---|---|---|---|---|
| eter | ness | Material | finish | Backing | PN |
| 20A | 0.010″ | Silicone | Super-soft | Plain | 86435K41 |
| 20A | 0.020″ | Silicone | Super-soft | Plain | 86435K45 |
| 40A | 0.020″ | FDA Compliant | Smooth? | Adhesive | 86915K22 |
| | | Silicone | | | |
| 35A | 0.010″ | Silicone | Super-soft | Plain | 86435K41 |
| 10A | 0.010″ | Silicone | Super-soft | Plain | 86435K41 |
| 40A | 0.031″ | Neoprene | Smooth? | Plain | 9455K75 |
| 30A | 0.031″ | Neoprene | Smooth? | Plain | 9455K75 |
| 40A | 0.031″ | Neoprene | Textured | Plain | 8445K31 |

The adhesiveness of the sheet to the tongue surface was qualitatively evaluated and recorded. Additionally, once the sheet was placed on the tongue surface, a gloved hand was placed on the sheet to tactilely feel for the tongue tissue under the sheet. For the tested designs that included a position indicator such as a hole or opening, the sheet was also evaluated for ease of determining the location of the position indicator, which included the degree tactile feedback available given the sheet thickness and material. Table 4 provides a summary of the qualitative observations from the testing. As shown, the plain textured Neoprene sheet (PN8445K31) showed promise for both evaluated categories.

TABLE 4
Silicone sheeting Testing

| | | | | | | | Can |
|---|---|---|---|---|---|---|---|
| | | | | | | | differentiate |
| | | | | | | Rating | between |
| | | | | | | (1 is worst, | tongue and |
| | | | | | | 3 is best) | material |
| | | | Surface | | Mcmaster | Sticks well to | using gloved |
| Durometer | Thickness | Material | finish | Backing | PN | the tongue | hand |
| 20A | 0.010″ | Silicone | Super-soft | Plain | 86435K41 | 3 | 1 |
| 20A | 0.020″ | Silicone | Super-soft | Plain | 86435K45 | 3 | 1 |
| 40A | 0.020″ | FDA | Smooth? | Adhesive | 86915K22 | 1 | 3 |
| | | Compliant | | | | | |
| | | Silicone | | | | | |
| 35A | 0.010″ | Silicone | Super-soft | Plain | 86435K41 | 2 | 1 |
| 10A | 0.010″ | Silicone | Super-soft | Plain | 86435K41 | 3 | 1 |
| 40A | 0.031″ | Neoprene | Smooth? | Plain | 9455K75 | 2 | 2 |
| 30A | 0.031″ | Neoprene | Smooth? | Plain | 9455K75 | 2 | 2 |
| 40A | 0.031″ | Neoprene | Textured | Plain | 8445K31 | 2 | 3 |

### Example 3

A cadaver lab was conducted to determine the feasibility of placing tongue implants using templates, stylets and dilator/sheath assemblies.

First Specimen:

For the first specimen, the exercise was to test various tip styles of the 0.037 inch Teflon coated stylets. The observations for each of the three tip designs are described in Table 5.

TABLE 5

| Stylet Tip | |
|---|---|
| Design | Observation |
| 20° taper, | The initial insertion required a little bit of force. Once started |
| .015″ | travel of the stylet to the base of the tongue was moderate. It |
| radius | was neither too easy nor too difficult. It was difficult to |
| | perforate the base of the tongue with this stylet. Tenting was |
| | visible but the tenting created was somewhat diffuse. |
| 20° taper, | Initial insertion was easier than the .015″ radius stylet. |
| .005″ | Travel was slightly easier and the resistance to perforation |
| radius | was about the same. Tenting was slightly better than with the |
| | .015″ radius version. |
| 45° bevel | Both insertion and travel were easier than the .005″ radius |
| | stylet. Tenting was more pronounced with the beveled |
| | version. Perforation was easier than with the radiused |
| | versions. |
| 60° bevel | Both insertion and travel were even easier than the 45° |
| | bevel. Tenting and perforation were about the same. |

Additionally, for the first specimen, after multiple stylets were inserted into the tongue, visual verification was carried out by using a mirror and a flexible endoscope to aid and verify placement of the stylets. With the mirror, the physician was able to visualize the placement of the lower stylets, which could not be seen without the mirror. However, the mirror offered challenges in needing to manually mark locations with a pen, which can be difficult to do in an operation setting. Visual tracking with the flexible endoscope was determined to be a possible tool to be used in conjunction with a tissue template to confirm depth and placement.

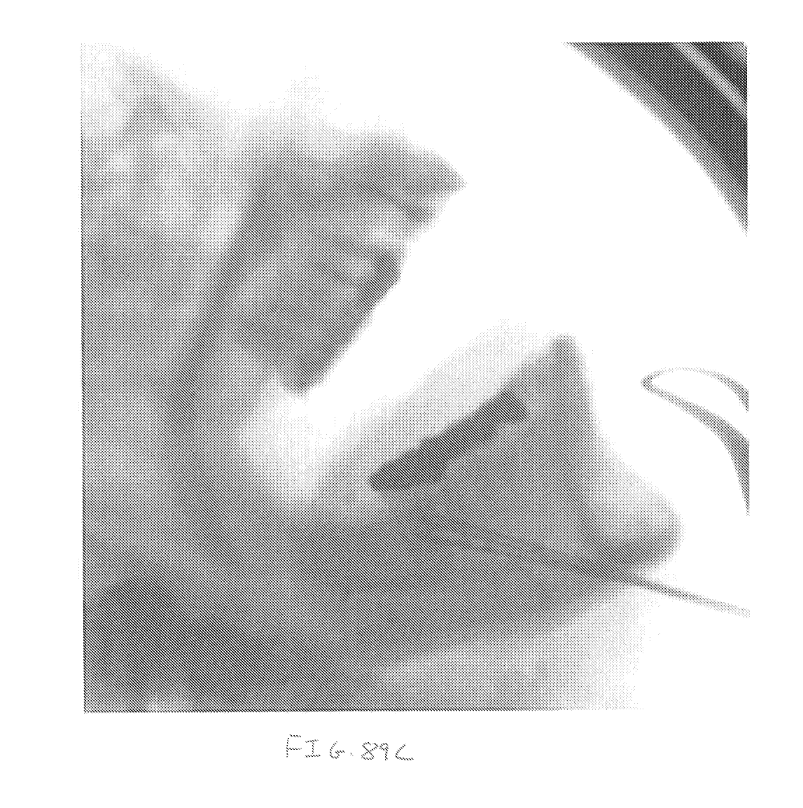

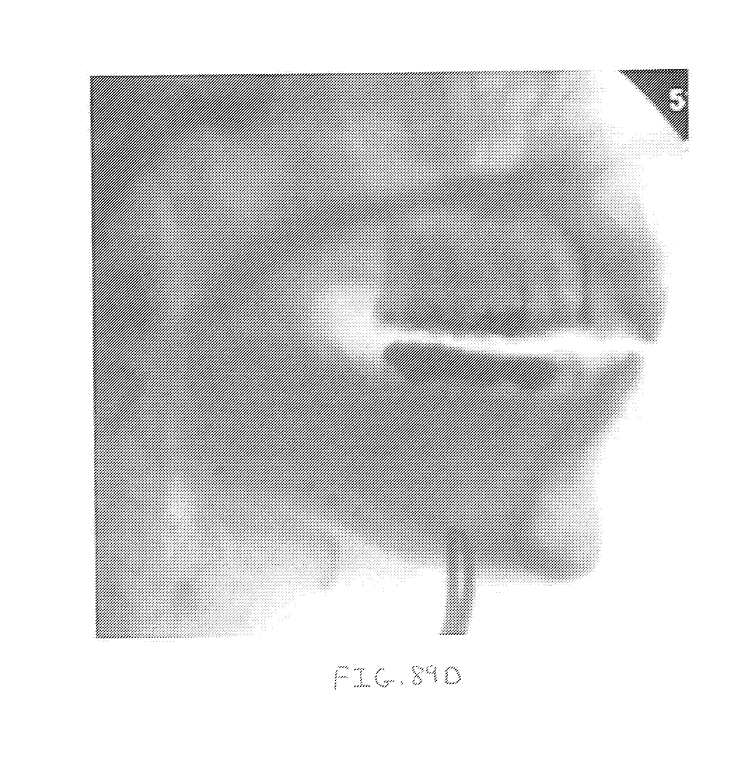

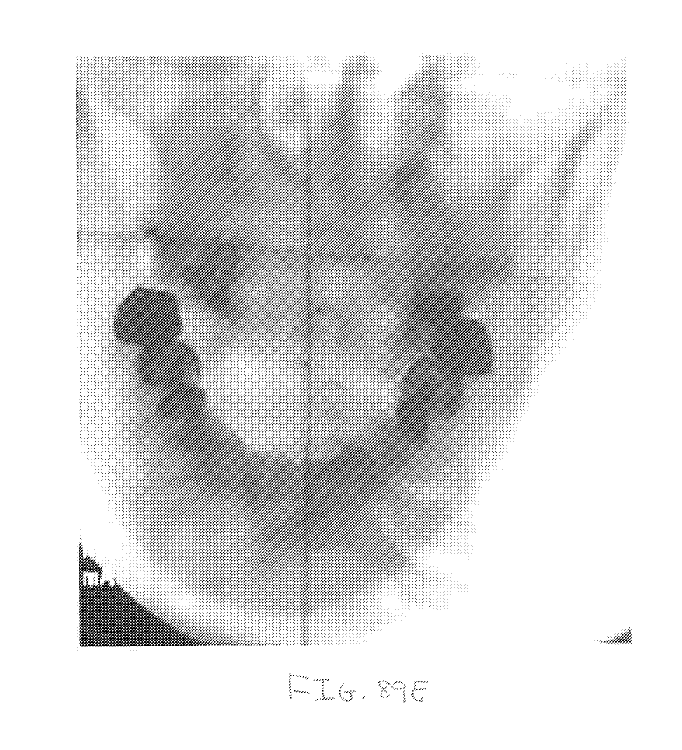

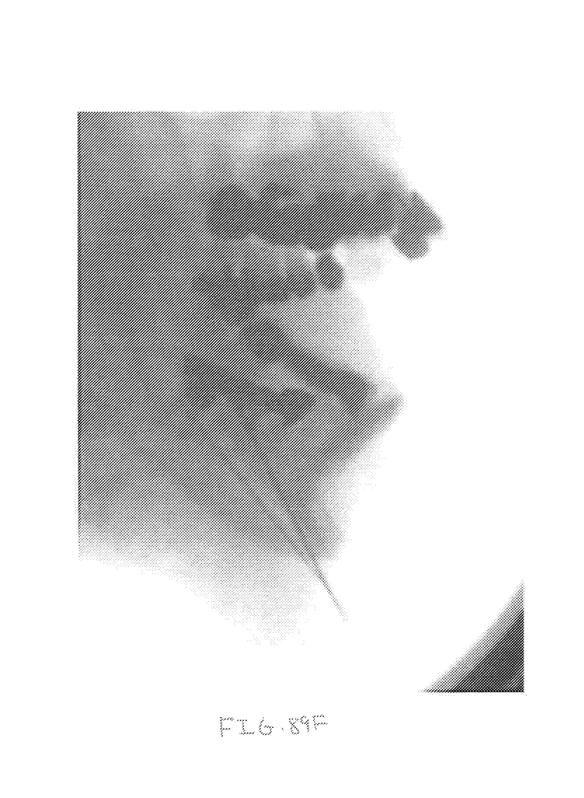

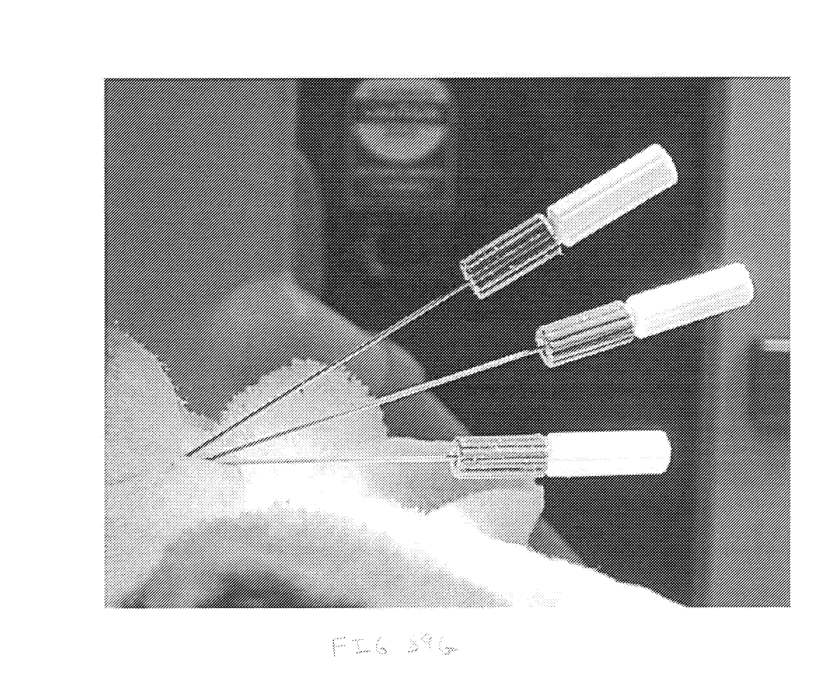

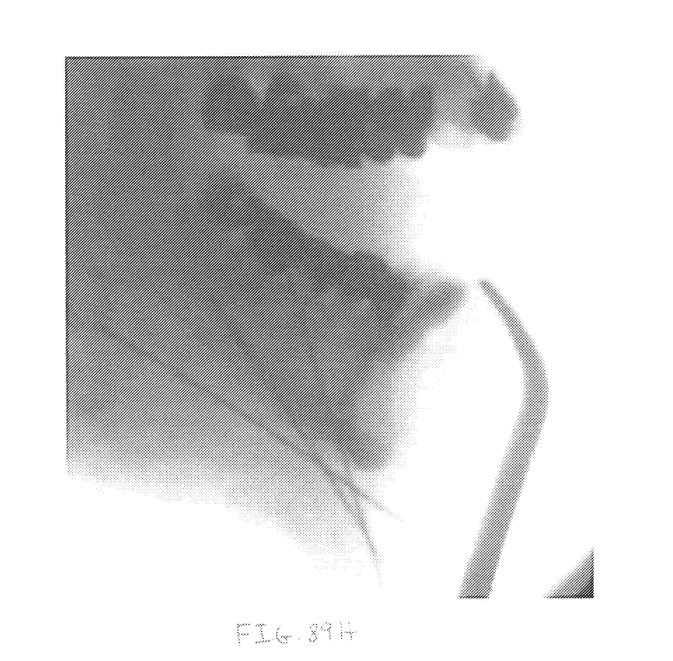

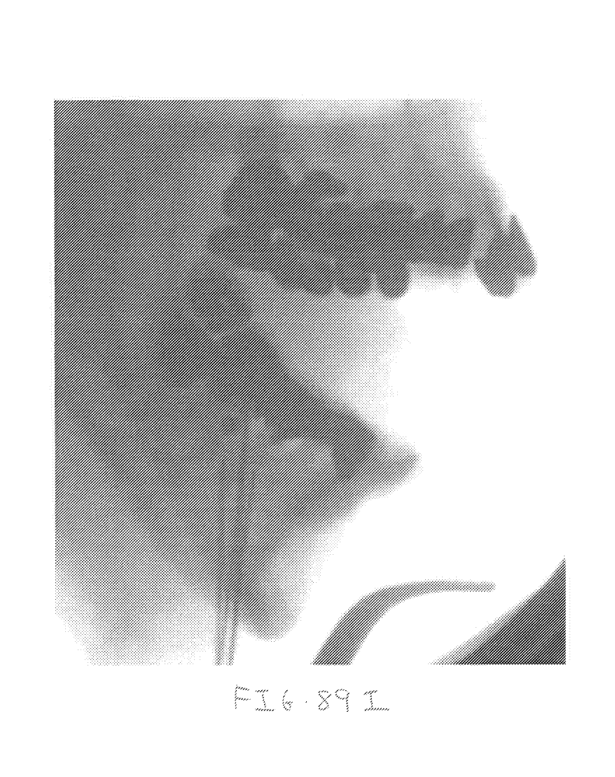

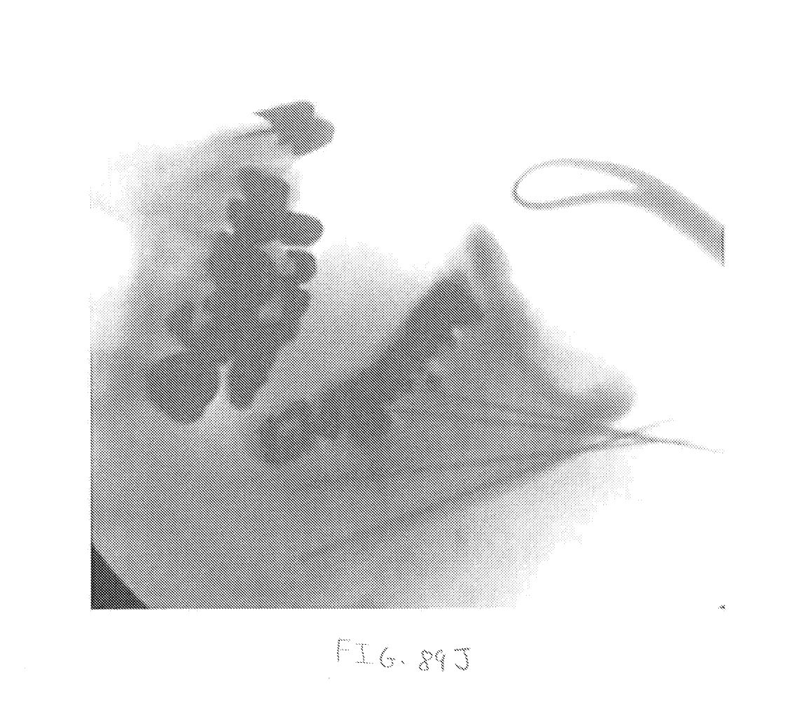

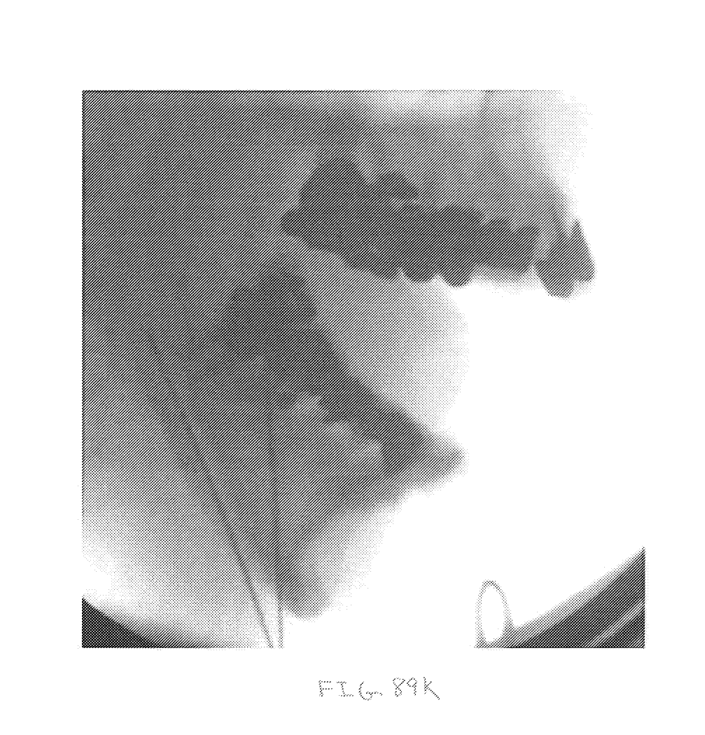

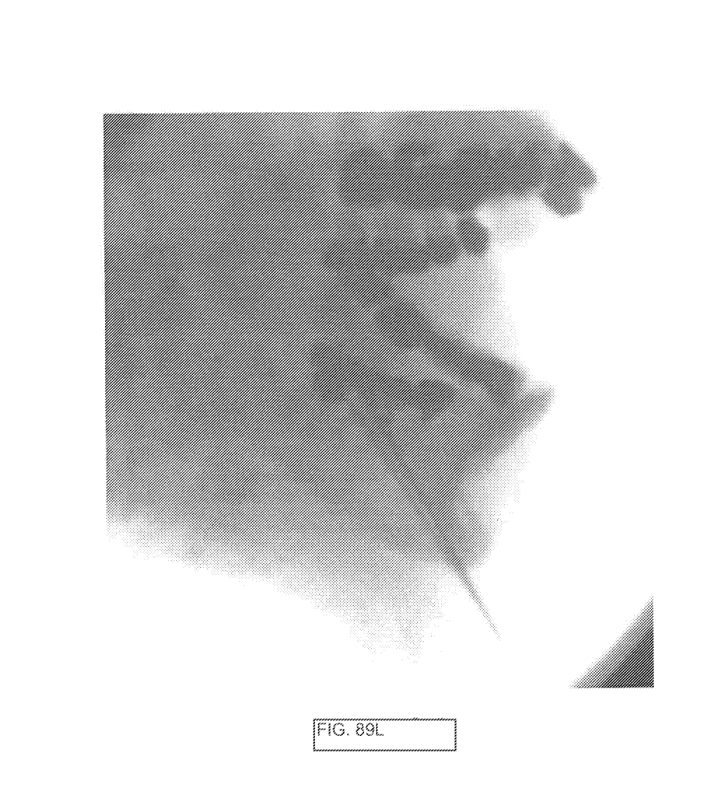

In placing the multiple stylets, the physician used two template designs. The first template design was a finger cot template with openings on the finger cot for indicating the target positioning of stylets when the fingers are placed on the cadaver tongue. The physician placed two stylets using the finger cot. The first stylet that he placed was not deep enough upon initial placement and had to be pushed in approximately 1-2 cm. The second stylet had good depth and the spread that was achieved was good. An observation was made that a thicker template, such as one with about 4 mm thickness, may work to provide improved tactile contrast. FIGS. 89A-B show two x-rays image of the stylets placed into the cadaver tongue using the finger cot template.

The second template design was a sheet of material adhered to the tongue to using tissue glue. Three stylets were inserted into the tongue using the glued template. An observation was made that the template may need to be better adhered to the tongue to avoid template movement. FIG. 89C shows an x-ray image of the three stylets in situ.

After placing the two stylets with the finger cot template, the physician used the stylets to deliver two implants into the tongue. A 8 Fr dilator and sheath assembly went over the stylet and to the base of the tongue easily. On the assemblies used there is a 2 cm difference between the tip of the dilator and the tip of the sheath. Because of this difference the sheath had to be uncoupled from dilator and advanced to the base of the tongue. However, this was also done easily. The depth and spread of the two implants that he placed were very good. The A-P view showed that midline placement was good as well. (FIGS. 89D-E)

Second Specimen:

Three stylets were placed into the second specimen. For the first attempt a finger cot template was used. This attempt resulted in a tight grouping of the three stylets (FIG. 89L). One of the stylets was removed and repositioned (FIG. 89F).

For the second attempt at placing three stylets, the physician used the external positioning of the stylets to judge the internal position of the stylets. This had a better result than the finger cot concept. The spread was too wide for what would be ideal. However, it was noted that the external stylet spread was representative of what was seen inside the tongue on fluoroscopy. FIG. 89G shows a photo of the external portion for each of the three stylets. FIG. 89H shows a fluoroscopic x-ray image with a lateral view of the three stylets of FIG. 89H. It was observed that the external portion of the stylets corresponded with the placement of the internal portions of the stylets. FIG. 89I shows a fluoroscopic x-ray image of a fourth stylet in place.

On the third attempt at placing stylets, four stylets were placed (FIG. 89J). An image was taken after two stylets were placed and the spacing looked good. However, when four stylets were placed they ended up in a tight grouping. The second two stylets were removed the desired spacing returned (FIG. 89K). It is possible that placing four wires leads to compressing/moving of the tongue which results in moving the initial wires closer together.

### Summary of Observations for Example 3

Stylets. The 0.037″ stylets proved to be sufficient for tunneling to the back of the tongue. The 0.018″ and 0.025″ stylets were not tested. Of the different tips that were tested the tapered 0.005″ radius tip provided the best combination of entry, travel, perforation and tenting. The beveled tips had better entry and travel but perforated too easily. It was found that the external stylet spread was representative of what was seen inside the tongue on fluoroscopy. It is able to achieve a good spread of stylets without the use of fluoroscopy.

Dilators. The 8 FR dilator/sheath assembly proved to work well. The sheath was able to reach the base of the tongue and the implants placed using the sheath had good depth. One issue with the assembly is the distance between the tip of the dilator and the tip of the sheath. The sheath has to be uncoupled from the dilator and advanced to the tongue base before deploying the implant. Part of this distance can be reduced by making a custom assembly.

Template. Use of the template to place multiple stylets may be improved by using a sufficient amount of adhesive and providing adequate cure time.

Depth. Early in the lab, the physician appeared to have issues pushing the stylets deep enough. After he became comfortable with the stylets, he easily pushed them deep enough. It was noted that sometimes the stylets backed-off a bit while inserting the other stylets, but it was easy to push them back to the correct depth before inserting the dilators. Additionally, depth can be assessed and modified after placement of all stylets because the physician can then use their fingers to palpate without accidently affecting placement due to tongue movement. The use of visual aids, mirror and flexible endoscope may be used to help verify placement.

Additionally, it was observed that the average peak insertion force for varied for a trocar, introducer (e.g. dilator and sheath assembly), and stylet. Table 6 shows these forces.

TABLE 6

| | Average Peak Insertion | |
|---|---|---|
| Design | Force in Cadaver (lbf) | Stdev (lbf) |
| Trocar | 2.28 | 0.57 |
| Stylet | 1.15 | 0.21 |
| Introducer (4 mm tip) | 1.05 | 0.39 |

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

Unless defined otherwise, all technical terms used herein have the same meanings as commonly understood by one of ordinary skill in the art to which this invention belongs. Specific methods, devices, and materials are described in this application, but any methods and materials similar or equivalent to those described herein can be used in the practice of the present invention. While embodiments of the inventive device and method have been described in some detail and by way of exemplary illustrations, such illustration is for purposes of clarity of understanding only, and is not intended to be limiting.

Various terms have been used in the description to convey an understanding of the invention; it will be understood that the meaning of these various terms extends to common linguistic or grammatical variations or forms thereof. It will also be understood that when terminology referring to devices or equipment has used trade names, brand names, or common names, that these names are provided as contemporary examples, and the invention is not limited by such literal scope. Terminology that is introduced at a later date that may be reasonably understood as a derivative of a contemporary term or designating of a subset of objects embraced by a contemporary term will be understood as having been described by the now contemporary terminology.

While some theoretical considerations have been advanced in furtherance of providing an understanding of the invention the claims to the invention are not bound by such theory. Described herein are ways that embodiments of the invention may engage the anatomy and physiology of the airway, generally by opening the airway during sleep; the theoretical consideration being that by such opening of the airway, the implanted device embodiments alleviate the occurrence of apneic events. Moreover, any one or more features of any embodiment of the invention can be combined with any one or more other features of any other embodiment of the invention, without departing from the scope of the invention. Further, it should be understood that while these inventive methods and devices have been described as providing therapeutic benefit to the airway by way of intervention in tissue lining the airway, such devices and embodiments may have therapeutic application in other sites within the body, particularly luminal sites. Still further, it should be understood that the invention is not limited to the embodiments that have been set forth for purposes of exemplification, but is to be defined only by a fair reading of claims that are appended to the patent application, including the full range of equivalency to which each element thereof is entitled.

What is claimed is:

1. A system for delivering an implant into a patient's airway tissue comprising:
at least one wire comprising a first wire having a first distal end, a first proximal end, and a first wire axis configured to define a first implant position in the airway tissue for a first implant, the first distal end configured to allow at least partial insertion of the first wire into the airway tissue, the first wire configured such that when the first wire is partially inserted into the airway tissue a proximal portion of the first wire remains outside the patient's body;
a wire guide comprising a second distal end, a second proximal end, and at least a first wire channel and a second wire channel, each wire channel having a proximal opening, a distal opening, and a lumen extending between the openings, the first and second wire channels each being configured to releasably receive and retain a wire, and the first wire channel having a first channel axis and the second wire channel having a second channel axis, wherein the first channel axis and second channel axis extend lengthwise through the first and second wire channels respectively.

2. The system of claim 1, wherein the first channel axis and the second channel axis define an angle between about 0 degrees to about 60 degrees.

3. The system of claim 1, wherein the wire guide is configured to receive a first wire in the first wire channel and a second wire in the second wire channel to thereby align the first wire and the second wire in a predetermined orientation.

4. The system of claim 1, wherein the at least one wire comprises a second, third, and fourth wire each configured for partial insertion into the airway tissue, and wherein the wire guide comprises a third wire channel configured to receive the third wire and a fourth wire channel configured to receive the fourth wire.

5. The system of claim 1, further comprising at least one light emitter on the first wire.

6. A system for delivering an implant into a patient's airway tissue comprising:
at least one wire comprising a first wire having a first distal end, first proximal end, and a first axis configured to define a first implant position in the airway tissue for a first implant, and the first distal end configured to allow at least partial insertion of the first wire into the airway tissue, wherein when partially inserted a proximal portion of the first wire is configured to remain outside the patient's body;
a sheath and dilator assembly comprising:
a dilator having a second proximal end, a second distal end, and a dilator lumen extending through the dilator between the second proximal and distal ends, wherein the first wire is configured to be movably positioned in the dilator lumen to guide the advancement of the sheath and dilator assembly into the airway tissue; and
a sheath having a third proximal end, a third distal end, and a sheath lumen extending between the third proximal and distal ends, the sheath configured to removably receive the dilator into the sheath lumen, wherein the second distal end of the dilator is configured to extend beyond the third distal end of the sheath as the sheath and dilator assembly is advanced into the airway tissue; and
a pusher having a fourth proximal end and a fourth distal end, the fourth distal end configured to releasably engage a distal portion of the first implant, wherein the pusher is configured to move through the sheath lumen and deploy the first implant in the first implant position in the airway tissue.

7. A system for delivering an implant into an airway tissue comprising:
a sheath and dilator assembly comprising:
a dilator having a proximal end, a distal end, a dilator shaft extending through the dilator between the proximal and distal ends, and a tip portion at the distal end of the dilator shaft, wherein the tip portion comprises an eccentric tip configured to guide the advancement of the sheath and dilator assembly through the airway tissue; and
a sheath having a proximal end, a distal end, and a sheath lumen extending between the proximal and distal ends, the sheath configured to removably receive the dilator into the sheath lumen, wherein the tip portion of the dilator extends beyond the distal end of the sheath as the sheath and dilator assembly is advanced into the airway tissue.

8. The system of claim 7, wherein the second distal end of the dilator is tapered.

9. The system of claim 7, wherein the eccentric tip defines a tip axis that is eccentric relative to a central longitudinal axis of the dilator, the tip axis being parallel to the central longitudinal axis of the dilator.

10. The system of claim 7, wherein the tip portion is configured to align the dilator with a midline plane of the patient.

11. A method of treating an airway disorder comprising:
creating an incision on a surface of a tissue near an airway forming tissue;
partially inserting at least a first wire and a second wire into the airway forming tissue through the incision, wherein a first axis of the first wire defines a first implant position for a first implant in the airway forming tissue and a second axis of the second wire defines a second implant position for the second implant in the airway forming tissue, the first axis and the second axis forming an angle between about 0 degrees to about 45 degrees;
placing a first implant at the first position in the airway forming tissue by guiding the first implant to the first implant position along a first path defined by the first axis of the first wire;
placing a second implant at the second position in the airway forming tissue by guiding the second implant along a second path defined by the second axis of the second wire; and
removing the first and second wires from the airway forming tissue.

12. The method of claim 11, wherein the first implant position and the second implant position form an angle between 0 degrees to about 45 degrees.

13. The method of claim 11, further comprising inserting at least the first and second wires into a wire guide positioned proximal to the incision, wherein the wire guide maintains an orientation of the first axis and the second axis relative to each other; and removing the first and second wires from the wire guide without removing the first and second wires from the incision.

14. A method of treating an airway disorder comprising:
creating an incision on a surface of a tissue near an airway forming tissue;
partially inserting a first wire and a second wire into the airway forming tissue through the incision, wherein a first axis of the first wire defines a first implant position for a first implant in the airway forming tissue and a second axis of the second wire defines a second implant position for the second implant in the airway forming tissue;
guiding an implant delivery device through the incision and at least partially into the airway forming tissue by advancing the delivery device along a length of the first wire;
placing a first implant at the first position in the airway forming tissue;
removing the implant delivery device from the airway forming tissue after placing the first implant in the first position;
guiding the implant delivery device through the incision and at least partially into the airway forming tissue by advancing the delivery device along a length of the second wire;
placing a second implant at the second position in the airway forming tissue; and
removing the implant delivery device from the airway forming tissue after placing the second implant in the second position.

15. The method of claim 14, further comprising releasably engaging an end of the first implant with a pusher; inserting the pusher through a lumen of the implant delivery device to deploy the first implant at the first position; releasably engaging an end of the second implant with a pusher; and inserting the pusher through a lumen of the implant delivery device to deploy the second implant at the second position.

16. A method of treating an airway disorder comprising:
creating an incision on a surface of a tissue near an airway forming tissue;
advancing a sheath and dilator assembly through the incision and at least partially into the airway forming tissue, wherein the dilator comprises an eccentric tip configured to guide the assembly along a curved area of the airway forming tissue;
placing a first implant at the first position in the airway forming tissue;
placing a second implant at the second position in the airway forming tissue; and
removing the assembly from the airway forming tissue.

17. The method of claim 16, wherein the eccentric tip is configured to position the tip portion of the dilator near a midline of the airway forming tissue.

18. A system for delivering an implant into a patient's airway tissue comprising:
at least one wire comprising a first wire having a first distal end, first proximal end, and a first axis configured to define a first implant position in the airway tissue for a first implant, and the first distal end configured to allow at least partial insertion of the first wire into the airway tissue, wherein when partially inserted a proximal portion of the first wire remains outside the patient's body; and
a sheath and dilator assembly comprising:
a dilator having a second proximal end, a second distal end, and a dilator lumen extending through the dilator between the second proximal and distal ends, wherein the first wire is configured to be movably positioned in the dilator lumen to guide the advancement of the sheath and dilator assembly into the airway tissue; and
a sheath having a third proximal end, a third distal end, and a sheath lumen extending between the third proximal and distal ends, the sheath configured to removably receive the dilator into the sheath lumen, wherein the second distal end of the dilator extends beyond the third distal end of the sheath as the sheath and dilator assembly is advanced into the airway tissue.

19. The system of claim 18 further comprising a pusher having a fourth proximal end and a fourth distal end, the fourth distal end configured to releasably engage a distal portion of the first implant, wherein the pusher is configured to move through the sheath lumen and deploy the first implant in the first implant position in the airway tissue.

20. The system of claim 18, wherein the at least one wire comprises a second wire.
